(19) United States
(12) Patent Application Publication
(10) Pub. No.: US 2026/0257039 A1
(43) Pub. Date: Sep. 3, 2026

(54) CONVECTION-ENHANCED THERMO-CHEMOTHERAPY CATHETER SYSTEM

(71) Applicant: Board of Regents, The University of Texas System, Austin, TX (US)

(72) Inventors: Christopher Rylander, Austin, TX (US); Jason Mehta, Austin, TX (US); Christopher Cook, Round Rock, TX (US); Egleide Elenes, Austin, TX (US)

(21) Appl. No.: 18/834,413

(22) PCT Filed: Feb. 13, 2023
(86) PCT No.: PCT/US2023/062458

§ 371 Date: Jul. 30, 2024

Related U.S. Application Data
(60) Provisional application No. 63/309,227, filed on Feb. 11, 2022.

(51) Int. Cl. A61M 25/01; A61B 5/00; A61B 5/055; A61B 17/00; A61M 25/00; A61N 5/06; A61N 5/067

(52) U.S. Cl. CPC A61M 25/0127; A61B 5/0036; A61B 5/0046; A61B 5/055; A61N 5/0625; A61N 5/067; A61B 2017/00292; A61B 2017/00911; A61M 2025/0085; A61M 2205/051; A61N 2005/0612; A61N 2005/063

(57) ABSTRACT

The present invention provides systems and methods for improved dispersal volume of fluid agents. The systems and methods are configured to maintain continuous movement of delivery lumens while simultaneously modulating flow rates of fluid agents to maintain controlled pressure to enhance dispersal volume. In some embodiments, the systems and methods are useful in improving drug distribution in target tissues such as chemotherapeutics in treating glioblastomas, such as by precision positioning of individual microneedles, tissue heating through light delivering fiber optic microneedles, and real-time observation of drug infusion through MRI monitoring.

### CROSS-REFERENCE TO RELATED APPLICATIONS

This application claims priority to U.S. Provisional Patent Application No. 63/309,227, filed on Feb. 11, 2022, incorporated herein by reference in its entirety.

### STATEMENT REGARDING FEDERALLY SPONSORED RESEARCH OR DEVELOPMENT

This invention was made with government support under Grant no. P01 CA207206 awarded by the National Institutes of Health. The government has certain rights in the invention.

### BACKGROUND OF THE INVENTION

Convection Enhanced Delivery (CED) is a method of delivering large molecules across the blood brain barrier and directly into cancer tissue. Previous clinical studies have not shown improved patient outcomes when using CED for glioblastoma treatment, possibly because of the failure of drugs to adequately cover the margins of a tumor.

Methods for increasing dispersal volume of drugs are numerous, but focus primarily on the fabrication of multiport catheters (Elenes EY et al., Journal of Engineering and Science in Medical Diagnostics and Therapy. 2021 Feb. 1; 4 (1): 011003; Elenes EY et al., Exon Publications. 2017 Sep. 20:359-72; Vogelbaum M A et al., Journal of neurosurgery. 2018 Apr. 13; 130 (2): 476-85) and preventing reflux (Gill T et al., Journal of neuroscience methods. 2013 Sep. 30; 219 (1): 1-9; Krauze MT et al., Journal of neurosurgery. 2005 Nov. 1; 103 (5): 923-9; Vazquez LC et al., Journal of Materials Science: Materials in Medicine. 2012 August; 23 (8): 2037-46; Yin D et al., Journal of neuroscience methods. 2010 Mar. 15; 187 (1): 46-51) which has been shown to be a large issue in the forward distribution of therapeutics with CED to targeted tissue regions (Chen MY et al., Journal of neurosurgery. 1999 Feb. 1; 90 (2): 315-20). Furthermore, no multiport catheters can be remotely actuated while being observed in real-time with MRI. Several drive systems for MRI compatible robots exist, but no purely mechanical drive systems are known. Constant pressure infusions are not typically used in clinical settings and when researchers do implement constant pressure infusions, they are under the assumption that constant pressure results in a constant flow rate, which is not always the case.

Therefore, there is a need in the art for improved systems and methods for convection enhanced delivery of fluid agents and for MRI compatible fluid delivery systems. The present invention addresses this need.

### SUMMARY OF THE INVENTION

In one aspect, the present invention relates to a controlled pressure, continuous movement fluid agent delivery system, comprising: one or more delivery lumen; one or more fluid agent reservoir fluidly connected to the one or more delivery lumen by a tubing or fluid line; one or more linear actuator mechanically connected to the one or more delivery lumen; one or more pressure sensor; one or more pump; and a controller; wherein the controller is configured to modulate a flow rate of a fluid agent by the one or more pump to maintain a controlled pressure in the one or more delivery lumen as measured by the one or more pressure sensor as the one or more linear actuator moves the one or more delivery lumen.

In one embodiment, the one or more delivery lumen comprises a fiberoptic needle configured to conduct a laser light. In one embodiment, the one or more linear actuator comprises a lead screw actuated by a stepper motor. In one embodiment, the one or more linear actuator comprises a Bowden cable. In one embodiment, the one or more pump is selected from the group consisting of: a diaphragm pump, a gear pump, a lobe pump, a peristaltic pump, a piston pump, a variable height fluid column, and a syringe. In one embodiment, the controlled pressure is constant as the one or more linear actuator moves the one or more delivery lumen.

In one aspect, the present invention relates to a method of controlled pressure, continuous movement fluid agent delivery, comprising the steps of: providing a fluid agent delivery system comprising one or more delivery lumen, each delivery lumen being connected to a linear actuator, a pressure sensor, a fluid agent reservoir, and a pump; positioning the one or more delivery lumen at a starting position; actuating the one or more delivery lumen from the starting position to an ending position by the linear actuator while the pump simultaneously administers a fluid agent at a flow rate that maintains a controlled pressure as measured by the pressure sensor; and ceasing movement of the one or more delivery lumen at the ending position.

In one embodiment, the method further comprises a step of ceasing administration of the fluid agent after a delay after the one or more delivery lumen reaches the ending position. In one embodiment, the delay is between about 1 second and 1 hour. In one embodiment, the one or more delivery lumen is actuated from the starting position to the ending position at a rate between about 0.1 mm/minute and 100 mm/minute. In one embodiment, the one or more delivery lumen is retracted from the starting position to the ending position. In one embodiment, the controlled pressure is below a critical pressure or reflux pressure. In one embodiment, the controlled pressure is constant during actuation of the one or more delivery lumen from the starting position to the ending position.

In one aspect the present invention relates to an MRI compatible convection-enhanced thermo-chemotherapy catheter system, comprising: a needle assembly comprising one or more needle fluidly connected to a reservoir by tubing or fluid lines, a pump, and a pressure sensor configured to measure pressure at the one or more needle; an actuating block comprising one or more linear actuator mechanically connected to the one or more needle; a motor bank mechanically connected to the one or more linear actuator; and a controller; wherein the controller is configured to modulate a flow rate of a fluid agent by the pump to maintain a controlled pressure in the one or more needle as measured by the pressure sensor as the one or more linear actuator moves the one or more needle.

In one embodiment, the one or more needle comprises a fiberoptic needle configured to conduct a laser light. In one embodiment, the one or more needle comprises a magnetic resonance visible coating. In one embodiment, wherein the needle assembly comprises a main cannula through which the one or more needle is extendable and retractable. In one embodiment, the main cannula is mechanically connected to at least a first positioning mechanism configured to control a rotation, an extension, and a retraction of the main cannula. In one embodiment, the main cannula is mechanically connected to at least a second positioning mechanism configured to control a trajectory angle of the main cannula.

In one embodiment, the motor bank comprises one or more stepper motor mechanically connected to the one or more linear actuator by one or more transmission rod, such that rotations of the one or more stepper motor are translated by the one or more transmission rod to the one or more linear actuator to move the one or more needle. In one embodiment, the system further comprises one or more junction box mechanically connected between the motor bank and the one or more linear actuator, such that the one or more junction box forms a bend in a transmission path between the motor bank and the one or more linear actuator. In one embodiment, each of the one or more transmission rod is removably connected to the system by a quick connect attachment. In one embodiment, the motor bank is positionable at a location remote from a magnetic field of an MRI machine. In some embodiments, a motor bank may be configured to translate linear motion from a position remote from the MRI magnetic field by any other means known in the art, for example using one or more Bowden cables, pneumatic, or hydraulic transmission systems.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts a flowchart of an exemplary method of fluid delivery with constant pressure and continuous movement.

FIG. 2 depicts a schematic of an exemplary MRI-compatible convection-enhanced thermo-chemotherapy catheter system (CETCS).

FIG. 3 depicts exemplary arborizing multiport catheters. (Left) A main cannula is shown with 6 microneedles configured with 5 in plane and 1 centered. (Right) A main cannula is shown with 9 microneedles configured in 3 levels of 3 needles each.

FIG. 4 depicts (left, top) exemplary gadolinium coated microneedles, (left, bottom) exemplary titanium coated microneedles, and (right) an MR image of an uncoated needle (control) and a gadolinium coated needle (1).

FIG. 5 depicts an exemplary lead screw mechanism with a lift arm holding a microneedle.

FIG. 6 depicts an exemplary cannula actuating block flexible tube, and main cannula.

FIG. 7 depicts an exemplary actuating block, with lines showing the convergence of the microneedles inside the block.

FIG. 8 depicts an exemplary cannula actuating mechanism and main cannula.

FIG. 9 depicts an exemplary cannula deployment mechanism configured to control trajectory and deployment. (a) Front side of catheter deployment mechanism, showing the angle adjustment knob, the white fiberglass rod that connects to the motor, the rail that converts the motor rotation to linear displacement, and the catheter. (b) Catheter deployment mechanism set at 90° to the horizontal surface. (c) Catheter deployment mechanism set at 45° from the horizontal surface. (d) Catheter deployment mechanism set at 0° from the horizontal surface.

FIG. 10 depicts an exemplary junction box (left) with quick connects (exploded view of quick connect in right image).

FIG. 11 depicts (left) an exemplary single gear of a miter gear junction box with an adjustable length horizontal rod passing through and (right) multiple miter gear junction boxes with adjustable length rods.

FIG. 12 depicts (left) an exemplary drive motor bank assembly and (right) a ceiling-mounted drive motor bank assembly positioned in an MRI equipment room with connected rods passing into an MRI imaging room.

FIG. 13 depicts an exemplary actuation mechanism connected to transmission rods.

FIG. 14 depicts an exemplary graphical user interface for a convection-enhanced thermo-chemotherapy catheter system (CETCS) control system.

FIG. 15 depicts the results of experiments demonstrating enhanced dispersal volume of fluid in gel media through continuous, controlled retraction of a needle. (A) Stationary infusion, (B) continuously retracting infusion, and (C) boxplot of dispersal volume (Va) for stationary and continuously retracting microneedles after 100 minute infusion of indigo carmine dye into 0.5% (wt./wt.) agarose gel. * p<0.001.

FIG. 16 depicts the results of experiments comparing volume dispersed (Va) for constant flow rate and constant pressure infusion at varying controlled catheter movement rates.

FIG. 17 depicts a diagram of a full shadowgraphy setup used for infusions in agarose gel brain tissue phantoms.

FIG. 18 depicts an RLC electronic circuit analogous to a hydraulic circuit of the disclosure, with catheter inertance represented by an inductor (I), catheter resistance represented by a resistor (R), extension tubing compliance represented by a capacitor (C), and driving volumetric flow rate represented by current source (Qo(t)).

FIG. 19 depicts a block diagram of a PID controller.

FIG. 20 depicts a table listing experimental results after 100 minute infusions into agarose gel (n=5) represented as mean±std.

FIG. 21 depicts a table listing rise time and time to maximum pressure for each catheter group (n=5) represented as mean±std.

FIG. 22 depicts plots of time varying means of (a) pressure and (b) flow rate of n=5 infusions for the stationary constant flow rate (Sta CF), 0.25 mm/min controlled retraction constant flow rate (0.25 mm/min CF), 0.5 mm/min controlled retraction constant flow rate (0.5 mm/min CF), stationary constant pressure (Sta CP), 0.25 mm/min controlled retraction constant pressure (0.25 mm/min CP), and 0.5 mm/min controlled retraction constant pressure (0.5 mm/min CP) catheters (note: error bars representing the standard deviation are shown at a period of 20 minutes for visual clarity, but pressure and flow rate were measured once per second).

FIG. 23 depicts a plot of time varying pressure for three excluded stationary constant flow rate infusions. Mean stationary constant flow rate is shown for reference.

FIG. 24 depicts plots of time varying flow rate and pressure for abnormal stationary constant flow rate infusion.

FIG. 25 depicts a diagram of the geometry used for finite element analysis of retracting catheter infusions (a) porous matrix, (b) initial infusion cavity, (c) infusion cavity/catheter surface), (d) outer tissue boundary), and (e) top surface.

FIG. 26 depicts a schematic of pressure application over time, where Surf. 1 represents the initial infusion cavity, Surf. n represents an arbitrary surface that becomes active within the first 5 minutes of infusion, Surf. m represents a surface that becomes active at 5 minutes into the infusion, and Surf. i represents an arbitrary surface that becomes active after 5 minutes of infusion.

FIG. 26 depicts images of infusion cavity showing applied effective pressure at 0 min, 2 min, 4 min, and 6 min into an infusion with corresponding infusion cavity lengths of 0 mm, 2 mm, 2.5 mm, and 3 mm respectively and using a catheter retraction rate of 0.25 mm/min.

FIG. 28 depicts (a) V_{d} of constant pressure experiment shown for catheters moving at a rate of 0, 0.25, and 0.5 mm/min plotted with V_{d} predicted by the respective baseline computational models, and (b) flow rate of constant pressure experiment for catheters moving at a rate of 0, 0.25, and 0.5 mm/min plotted with flow rate predicted by the respective baseline computational models (error bars represent the 95% confidence interval of the experimental results for n=5 replicates).

FIG. 29 depicts (a) V_{d} of constant pressure experiment shown for catheters moving at a rate of 0, 0.25, and 0.5 mm/min plotted with V_{d} predicted by the respective adjusted computational models, and (b) flow rate of constant pressure experiment for catheters moving at a rate of 0, 0.25, and 0.5 mm/min plotted with flow rate predicted by the respective adjusted computational models (error bars represent the 95% confidence interval of the experimental results for n=5 replicates).

FIG. 30 depicts plots of V_{d} of the model against the experiment at 100 minutes with a best fit line with a slope of 1 and γ-intercept of 0 with catheters moving at a rate of 0 mm/min, 0.25 mm/min, and 0.5 mm/min for the (a) adjusted constant pressure and (b) constant flow rate models.

FIG. 31 depicts V_{d} of constant flow rate experiment shown for catheters moving at a rate of 0, 0.25, and 0.5 mm/min plotted with V_{d} predicted by the respective constant flow rate computational model (error bars represent the 95% confidence interval of the experimental results for n=5 replicates).

FIG. 32 depicts computational model results for catheter movement rates ranging from 0 to 1 mm/min showing the (a) volume dispersed (Va) at 100 minutes for all 3 models and (b) average flow rate for the constant pressure models.

FIG. 33 depicts V_{d} at 100 minutes for total movement distances ranging from 5 mm to 25 mm for the (a) adjusted constant pressure model and (b) constant flow rate model.

FIG. 34 depicts infusion profiles for various retraction rates with a concentration threshold of 0.05% for (a) adjusted constant pressure and (b) constant flow rate models.

FIG. 35 depicts (a and c) Volume dispersed (Va) after 100 minute infusions calculated with different normalized effective concentration (En) threshold levels for catheter retraction rates varying from 0 to 1 mm/min for the adjusted constant pressure model and constant flow rate model respectively, (b and d) percent change in V_{d} when changing the concentration threshold for the adjusted constant pressure model and constant flow rate model, respectively.

FIG. 36A depicts a schematic showing radial lines for horizontal concentration and vertical concentration at the radial midpoint of the infusion cloud, (FIG. 36B and FIG. 36D) radial concentration plot for adjusted constant pressure model and constant flow rate model, respectively, (FIG. 36C and FIG. 36E) vertical concentration plot for adjusted constant pressure model and constant flow rate model, respectively (cross symbol represents where the infusion cavity and pressure begins to decrease, diamond symbol represents where the infusion cavity and pressure reaches its minimum).

FIG. 37 depicts a cannula fabrication process. (a) Five evenly-spaced holes made in the side of PEEK tube using jig (1) and Dremel mill bit (2). (b) Five pieces of polyimide tube inserted through the holes, making an overlapping pattern, with a sixth tube positioned in the center of the other tubes. (c) Side-view of jig placed at the end of the PEEK tubing to hold the polyimide guide tubes in position. (d) Top-view of jig placed at the end of the PEEK tubing to hold the polyimide guide tubes in position. (e) Tube for flexible section added along with interface piece to connect to actuating block. (f) Main cannula tube filled with light-cured acrylic, excess guide tube material removed with blade, cannula tip filed to a point.

FIG. 38A depicts a complete main cannula attached to an actuating block.

FIG. 38B is a line drawing of an exemplary system comprising a main cannula and an actuating block.

FIG. 38C shows a detail view of a distal end of a main cannula of a system as disclosed herein.

FIG. 39 depicts microneedle fabrication. (a) Microneedle components: additively manufactured attachment piece, Luer lock plastic needle, PEEK tube for reinforcement, and glass capillary tube. (b) Assembled microneedle,

FIG. 40 depicts a microneedle actuation mechanism. (a) Lift arm with detached microneedle. (b) Lift arm and drive screw, with microneedle attached. (c) Complete needle actuation block with main cannula attached.

FIG. 41 depicts a schematic of a system layout. Major components include rotating rods (shown in red), actuation mechanisms (1), scanner level junction box (2), ceiling level junction box (3), and motor bank (4).

FIG. 42 depicts mechanisms for bends in the system. (a) Components of gear box section. (b) Assembled gear box section. (c) Fully assembled right turn junction in test system.

FIG. 43 depicts CAD representations of quick connects for ceiling attachments. A) Disconnected. B) Connected.

FIG. 44 depicts junctions for adjustable length rods. (a) Single gear pair of lower junction box with adjustable length horizontal rods. (b) Fully assembled junction. (c) PVC junction stand for test system.

FIG. 45 depicts a motor bank assembly. (a) Motor bank layout of motors. (b) Motor bank, side view. (c) Motor bank, top view.

FIG. 46 depicts an electronics box. (a) Open box showing microcontroller (1), stepper motor drivers (2), 12-V power supply (3), and exhaust fan (4). (b) Electronics box installed below motor bank in the test system.

FIG. 47 depicts a schematic of hysteresis. Symmetric hysteresis, with asterisks showing test points used to measure backlash (b1 and b2) at either end of the movement.

FIG. 48 depicts (a) Front and (b) side view of experimental setup. Note: Shown here are cannula movement mechanisms (1), microneedle actuation block (2), acrylic background (3), and lamp for backlighting (4).

FIG. 49 depicts an experimental setup for microneedle deployment measurement. (a) Clamp for side-deploying microneedle. (b) Clamp for center-deploying microneedle.

FIG. 50 depicts a sequence of image processing (showing cropped images). (a) Original image. (b) Image corrected for lens distortion, resulting in only very minor effects at the image edges. (c) Grey scale image. (d) Black and white image.

FIG. 51 depicts sample processed images through four steps of microneedle deployment. Images cropped to better show microneedle.

FIG. 52 depicts sample data set of microneedle deployment and retraction through the first two cycles.

FIG. 53 is a table listing backlash amount in the microneedles.

FIG. 54 depicts the effect of guide tube position on backlash: (a) Distribution of backlash values related to particular microneedle used. Plot shows guide tube positions with two different microneedles used in each position.; (b) Distribution of backlash values related to particular microneedle used. Plot shows particular microneedles used in two different guide tube paths. The guide tube path was shown to have greater impact on backlash than individual microneedle.

FIG. 55 depicts distribution of backlash values with different main cannula orientation.

FIG. 56 depicts the results of three repetitions of the third experiment (microneedles in their rotated positions and the cannula rotated to align with the camera).

FIG. 57 depicts the distribution of backlash values related to microneedle deployment angle. Set 1 represents the microneedles in their original positions and the main cannula rotated to align with the camera. Set 2 represents the microneedles in their original positions and the camera rotated about the stationary cannula. Set 3 represents the microneedles in their rotated positions and the cannula rotated to align with the camera.

FIG. 58 depicts input and output position changes at either end of transmission system for a side deploying microneedle.

FIG. 59 shows an image illustrating results of a retrospective study.

FIG. 60A shows an exemplary CETCS catheter with microneedles deployed 2 cm from the tip of the cannula.

FIG. 60B shows a sharp, conical tip of a cannula with microneedles retracted

FIG. 60C shows an individual silica microneedle (arrow points at the sharp-beveled tip).

FIG. 60D shows a catheter inserted through a prototype cranial PGP.

FIG. 60E shows a single co-delivery microneedle prototype.

FIG. 60F shows a schematic cross-section of a co-delivery microneedle design.

FIG. 61A shows a CT guided insertion of arborizing catheter in explanted canine brain.

FIG. 61B shows a spatial distribution of infusion volume (at the end of infusion time) seen in MR images for same catheter shown in FIG. 61A. Note: reflux is arrested at transition from microneedles to cannula.

FIG. 62A-FIG. 62C are CT images of distribution of iohexol in explanted porcine brain. FIG. 62A shows voxels with grayscale value corresponding to >10% of iohexol concentration selected to derive V_{d} for the single-port catheter (blue) and arborizing catheter (red). The volume of solution that leaked into the ventricles was segmented into a separate mask (green).

FIG. 62B is a volumetric rendering of the brain and V_{d} for each group.

FIG. 62C is a volumetric rendering showing infusion volumes for the single-port catheter and the arborizing catheter after removing ventricular leakage from image. The volume dispersed (Va) of the contrast agent was quantified for each catheter. V_{d} for the arborizing catheter was significantly higher than for the single-port catheter, 2235.8+569.7 mm³ and 382.2=243.0 mm³, respectively (n=7).

FIG. 63A shows temperature measurements evenly spaced from t=10⁻⁶⁰ mins. Sampling duration is for approximately 15 seconds in length.

FIG. 63B-FIG. 63G show that for laser energy of 100 mW, brain tissue damage is mechanical and limited to the trajectory of microneedle insertion (seen in FIG. 63E, FIG. 63C, and FIG. 63F) Some photothermally induced necrosis of cortical and subcortical structures is shown, which become more severe at higher laser power (See FIG. 63D and FIG. 63G).

FIG. 64A-FIG. 64C show fluorescent brain slices demonstrating larger LR dispersions (yellow-gold regions) associated with laser co-delivery (FIG. 64B, FIG. 64C) compared to infusion only and an untreated control (FIG. 64A).

FIG. 64D shows quantitative analyses of the effects of laser co-delivery on dispersed infusate volume, V_{d}, in brain specimens.

FIG. 65A shows a schematic of a CETCS with a remote control system. FIG. 65B shows an MR-compatible microneedle and cannula actuation mechanisms (blue housings) controlled by electromechanical stepper motors (not shown) via fiberglass rods (white).

FIG. 66A and FIG. 66B show V_{d} (FIG. 66A) and Flow Rate (FIG. 66B) of a constant pressure experiment shown for a single microneedle retracting at a rate of 0, 0.25, and 0.5 mm/min plotted with predicted values from computational models. Error bars represent the 95% confidence interval of the experimental results for n=5 replicates.

FIG. 67 shows CETCS delivery of QUAD conjugates+Gd-alb to a canine patient with spontaneous MG. The tip of the CETCS cannula is visible in the top left-hand corner of the parasagittal reconstructions. These images show the early and individually distinctive infusion distributions from each of the three needles deployed, and also identify the individual microneedles. Eventually, over a period of 49 minutes there was overlapping infusion distributions from each of the microneedles as intended.

FIG. 68A shows a co-registered kinematic model of CETCS cannula and microneedles (n=3) for initial treatment, 3D geometric model of target tumor (red), and Gd-alb distribution following initial treatment (yellow). FIG. 68B shows recommended repositioning of a CETCS catheter using novel software. Note primary cannula is inserted toward tumor, rotated 10°, and n=2 microneedles are redeployed.

FIG. 69 shows a process flow diagram of an exemplary adaptive treatment strategy using CETCS.

### DETAILED DESCRIPTION

The present invention provides systems and methods for improved dispersal volume of fluid agents. The systems and methods are configured to maintain continuous movement of delivery lumens while simultaneously modulating flow rates of fluid agents to maintain constant pressure to enhance dispersal volume. In some embodiments, the systems and methods are useful in improving drug distribution in target tissues such as chemotherapeutics in treating glioblastomas, such as by precision positioning of individual microneedles, tissue heating through light delivering fiber optic microneedles, and real-time observation of drug infusion through MRI monitoring.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

“Distal” as used herein refers to the bottom end of a device remote from point of attachment or origin. In disclosed embodiment, distal refers to the end furthest away from a medical professional when introducing a device in a patient. “Proximal” as used herein refers to the closest end of a device situated nearer to the center of the body or the point of attachment. In disclosed embodiments, proximal refers to the end closest to a medical professional when placing a device in the patient.

“Lumen” as used herein refers to a canal, duct or cavity within a tubular structure, or a tubular structure containing a canal, duct, or cavity.

“Infusion” as used herein refers to a process of slow introduction of an element, for example a solution, into or onto a target.

“Internal anatomical space” as used herein refers to any region and/or site that exist below external skin layer. An internal anatomical space may comprise a cavity and/or a cellular structure.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

Fluid Agent Delivery Systems and Methods with Controlled Pressure and Continuous Movement

The present invention includes systems and methods for fluid agent delivery, wherein the fluid agents are delivered with a controlled pressure while the delivery system is continuously moved, thereby enhancing dispersal volume and distribution of a fluid agent. In some embodiments, the systems comprise a controller configured to couple controlled pressure delivery with controlled system movement, such as a proportional-integral-derivative (PID) controller.

In some embodiments as disclosed herein, a “controlled pressure” means that for the duration of fluid agent delivery, a control system is tuned to maintain a pressure value measured for example at the point of delivery as close as possible to a target pressure value. In constant pressure applications, the target pressure value is the same for the duration of the fluid agent delivery. In other embodiments, the target pressure value may be varied over time. In one embodiment, the target pressure value may be set at a high value at the beginning of a treatment, in effect creating a short-duration high-pressure burst at the beginning of fluid agent delivery, and then ramping down to a constant target pressure for the remainder of the treatment duration. In some embodiments, such a high pressure burst at the beginning of a fluid agent delivery may help to dislodge clogs in one or more lumens, for example due to coring of the target material, for example human tissue, caused during insertion of the lumen. Although certain treatments, systems, and methods may be presented herein with one type of controlled pressure, for example a constant pressure, it is understood that the disclosed methods and systems may be interchangeably used for any pressure control scheme.

An exemplary fluid agent delivery system comprises one or more delivery lumen, linear actuators, pressure sensors, pumps, and fluid agent reservoirs. The one or more delivery lumen can comprise any suitable lumen, including but not limited to needles, catheters, and the like. In some embodiments, the one or more delivery lumen is integrated into a multi-lumen delivery instrument, such as an arborizing catheter as described elsewhere herein and in U.S. Pat. No. 10,220,124, the contents of which are incorporated by reference herein in its entirety. In some embodiments, the one or more delivery lumen comprises a fiberoptic needle, such that a laser light can be conducted through each delivery lumen.

The linear actuator is mechanically connected to the one or more delivery lumen to move the delivery lumens through a delivery site between a starting position and an ending position. Contemplated linear actuators include but are not limited to hydraulic, pneumatic, and electro-mechanical linear actuators. In some embodiments, the linear actuator comprises a lead screw actuated by a stepper motor. In certain embodiments, each delivery lumen is connected to a separate linear actuator, such that each delivery lumen is individually movable by its own linear actuator. In some embodiments, the linear actuator may comprise a Bowden cable, or a cable slidably positioned within a flexible sheath and configured to translate linear motion from one end of the sheath to the other.

Fluid agent reservoirs contain one or more desired fluid agents. Pumps are provided to transport the fluid agents from the reservoirs to the delivery lumens. Contemplated pumps include but are not limited to diaphragm pumps, gear pumps, lobe pumps, peristaltic pumps, and piston pumps. In some embodiments, a pump is integrated into a reservoir, such as in a syringe. Each reservoir is fluidly connected to the one or more delivery lumen by a tube or fluid line. In some embodiments, a pump may comprise a variable height fluid column fluidly connected to the delivery lumen, whose height for example can be moved up and down to adjust the pressure head of the fluid column, thereby creating a controlled pressure at an outlet of the delivery lumen. Each tube or fluid line connected to a delivery lumen comprises a pressure sensor configured to measure a fluid pressure within a delivery lumen.

The fluid agent delivery system is thereby configured to deliver one or more fluid agents from a fluid agent reservoir to a delivery site by way of one or more delivery lumens. A linear actuator mechanically connected to a delivery lumen is configured to position a delivery lumen at a delivery site in preparation for fluid agent administration. The system is configured to simultaneously administer a fluid agent at a flow rate using a pump and to continuously move the delivery lumen for the at least a portion of the duration of fluid agent administration. A controller is provided to modulate flow rate from the pump based on pressure readings from the pressure sensor, such that a constant pressure is maintained for the duration of fluid agent administration. In some embodiments, heat is applied for at least part of the duration of fluid agent administration, such as through a laser light conducted through a fiberoptic delivery lumen.

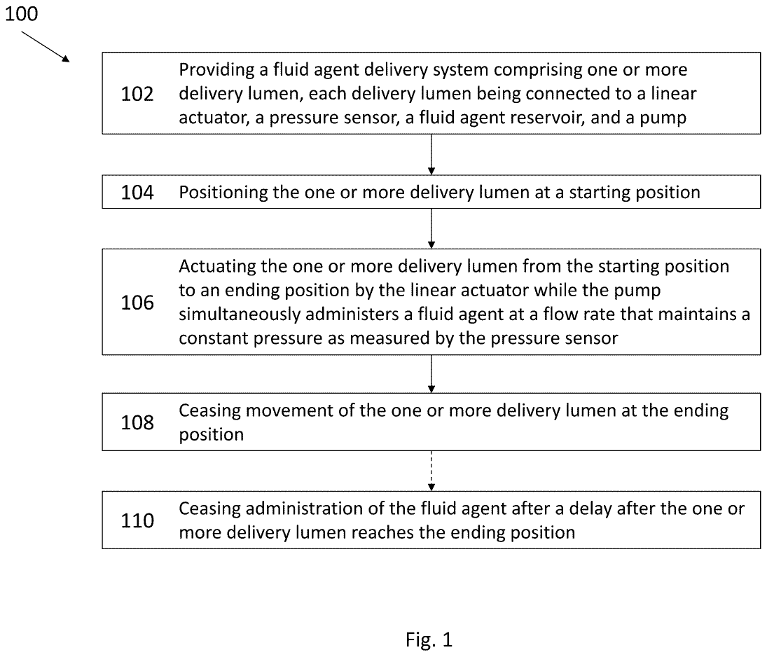

The above steps can be described in terms of exemplary method 100, shown in FIG. 1. Method 100 begins with step 102, wherein a fluid agent delivery system is provided, the fluid agent delivery system comprising one or more delivery lumen, each delivery lumen being connected to a linear actuator, a pressure sensor, a fluid agent reservoir, and a pump. In step 104, the one or more delivery lumen is positioned at a starting position. In step 106, the one or more delivery lumen is actuated from the starting position to an ending position by the linear actuator while the pump simultaneously administers a fluid agent at a flow rate that maintains a constant pressure as measured by the pressure sensor. In step 108, movement of the one or more delivery lumen is ceased at the ending position. Optionally in step 110, administration of the fluid agent is ceased after a delay after the one or more delivery lumen reaches the ending position.

The one or more delivery lumen is moved between the starting position and the ending position at a rate controlled by the linear actuator. As would be understood by persons having skill in the art, desired movement rates may be selected depending on factors such as fluid agent viscosity, fluid agent stability, fluid agent volatility, delivery site fragility, delivery site sensitivity, distance between the starting position and the ending position, and the like. In some embodiments, the movement rate is between about 0.1 mm/minute and 100 mm/minute. In various embodiments, the movement rate is about 0.1 mm/minute, 0.2 mm/minute, 0.3 mm/minute, 0.4 mm/minute, 0.5 mm/minute, 0.6 mm/minute, 0.7 mm/minute, 0.8 mm/minute, 0.9 mm/minute, or 1 mm/minute.

For the purposes of illustration, movement of a delivery lumen from a starting position to an ending position is described herein in a proximal direction, such that a delivery lumen is retracted from the starting position. However, it should be understood that movement of a delivery lumen can be in any desired direction. As a delivery lumen is retracted from a starting position towards an ending position, a fluid agent may be delivered at a constant flow rate. However, the present invention is based in part on the surprising and unexpected discovery that delivering a fluid agent at a variable flow rate that maintains a constant pressure leads to a greater dispersal volume of the fluid agent. As would be understood by persons having skill in the art, desired constant pressures may be selected depending on factors similar to those recited above. In some embodiments, a selected constant pressure is below a critical pressure or reflux pressure. A critical pressure or reflux pressure is defined as a threshold wherein exceeding the pressure causes an administered fluid agent to flow along the interface of the delivery needle and the tissue, opposite the flow of the administered fluid agent in the delivery lumen, and in some embodiments out of the delivery region of interest. Accordingly, a critical pressure or reflux pressure may be determined by increasing pressure of fluid agent delivery until reflux is observed. A critical pressure or reflux pressure may be experimentally determined prior to administration or determined in situ at a delivery site, such as at a starting position. In some embodiments, measured changes in pressure are useful in diagnosing issues in a fluid agent delivery system. For example, a high measured pressure could indicate a clogged delivery lumen, especially with no observable reflux.

In certain embodiments, administration of a fluid agent is ceased at the same time as movement of a delivery lumen is ceased. In some embodiments, administration of a fluid agent continues for a period of time after movement of the delivery lumen is ceased, such that there is a delay in cessation of fluid agent administration after movement of a delivery lumen is ceased. For example, ceasing both administration and movement at the same time could lead to insufficient time for the fluid agent to disperse through an ending position. Accordingly, cessation of fluid agent administration can be delayed by a period of time, such as about 1 second, 5 seconds, 10 seconds, 30 seconds, 1 minute, 5 minutes, 10 minutes, 30 minutes, 1 hour, and the like.

Fluid agents can be selected based on the delivery site. For example, in some embodiments fluid agents can comprise adhesives, cements, resins, glues, and the like for repairing cracked or damaged delivery sites such as wood, concrete, fiberglass, and the like. In some embodiments, fluid agents can comprise specimens such as cells, bacteria, spores, and the like for seeding delivery sites such as scaffolds and growing media.

In some embodiments, fluid agents are selected for a therapeutic application. For example, in some embodiments fluid agents can comprise drugs or medicaments for treating diseases or disorders in tissues such as the brain, muscle, liver, kidney, lung, intestine, skin, bone, and the like.

In some embodiments, fluid agents comprise bioactive molecules that recruit, attract or destroy cancer cells. For example, the bioactive molecules can include one or more additional extracellular matrix material and/or blends of naturally occurring extracellular matrix material, including but not limited to collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. In various embodiments, the one or more bioactive molecules can include one or more carbohydrates such as chitin, chitosan, alginic acids, and alginates such as calcium alginate and sodium alginate. These materials may be isolated from plant products, humans or other organisms or cells or synthetically manufactured.

In some embodiments, the bioactive molecules can include nucleic acids, such as mRNA and DNA. In some embodiments, the bioactive molecules can include natural or synthetic steroids and hormones, such as dexamethasone, hydrocortisone, estrogens, and its derivatives. In some embodiments, the bioactive molecules can include fibroblast growth factor (FGF), transforming growth factor beta (TGF-β), epidermal growth factor (EGF), VEGF (vascular endothelial growth factor), chemokines and modulators of the immune system, and modulators of angiogenesis. Further examples can include IGF-1 (insulin-like growth factor-1), IGF-2 (insulin-like growth factor-2), PDGF (platelet derived growth factor), RANTES, SDF-1, secreted frizzled-related protein-1 (SFRP-1), small inducible cytokine A3 (SCYA3), inducible cytokine subfamily A member 20 (SCYA20), inducible cytokine subfamily B member 14 (SCYB14), inducible cytokine subfamily D member 1 (SCYD1), stromal cell-derived factor-1 (SDF-1), thrombospondins 1, 2, 3 and 4 (THBS1-4), platelet factor 4 (PF4), lens epithelium-derived growth factor (LEDGF), midikine (MK), macrophage inflammatory protein (MIP-1), moesin (MSN), hepatocyte growth factor (HGF, also called SF), placental growth factor, IL-1 (interleukin-1), IL-2 (interleukin-2), IL-3 (interleukin-3), IL-6 (interleukin-6), IL-7 (interleukin-7), IL-10 (interleukin-10), IL-12 (interleukin-12), IFN-α (interferon-α), IFN-γ (interferon-γ), TNF-α (tumor necrosis factor-α), SDGF (Schwannoma-derived growth factor), nerve growth factor, neurite growth-promoting factor 2 (NEGF2), neurotrophin, BMP-2 (bone morphogenic protein 2), OP-1 (osteogenic protein 1, also called BMP-7), keratinocyte growth factor (KGF), interferon-γ inducible protein-20, and HIV-tat-transactivating factor, amphiregulin (AREG), angio-associated migratory cell protein (AAMP), angiostatin, betacellulin (BTC), connective tissue growth factor (CTGF), cysteine-rich angiogenic inducer 61 (CYCR61), endostatin, fractalkine/neuroactin, or glial derived neurotrophic factor (GDNF), GRO2, hepatoma-derived growth factor (HDGF), granulocyte-macrophage colony stimulating factor (GMCSF), endostatin, angiostatin, and peptide analogs or mimetics thereof.

In some embodiments, the bioactive molecules are selected from the group consisting of: transglutaminase (e.g., TGM1, TGM2, TGM3, TGM4, TGM5, TGM6, TGM7, F13A1), integrin (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, ITGB8), legumain (LGMN), lipocalin (e.g., LCN1, LCN2, LCN3, LCN4, LCN5, LCN6, LCN8, LCN9, LCN10, LCN11, LCN12, LCN15, LCN16, LCN17), growth arrest and DNA damage (e.g., GADD45A, GADD45B, GADD45G), histone protein (e.g., histone H1, histone H2A, histone H2B, histone H3, histone H4), Sr_{c} kinase-associated phosphoprotein (e.g., SKAP1, SKAP2), flotillin (e.g., FLOT1, FLOT2), Collagen I, Collagen V, Collagen XI, fibroblast activation protein (FAP), a disintegrin and metalloproteinase (e.g., ADAM1, ADAM2, ADAM7, ADAM8, ADAM9, ADAM10, ADAM11, ADAM12, ADAM15, ADAM17, ADAM18, ADAM19, ADAM20, ADAM21, ADAM 22, ADAM23, ADAM28, ADAM29, ADAM30, ADAM33), fibrillin (e.g., FBN1, FBN2, FBN3, FBN4), suppressor of cytokine signaling (e.g., SOCS1, SOCS2, SOCS3, SOCS4, SOCS5, SOCS6, SOCS7), signal-transducing adaptor protein (e.g., STAP1, STAP2), S100 proteins (e.g., S100A1, S100A2, S100A3, S100A4, S100A5, S100A6, S100A7, S100A8, S100A9, S100A10, S100A11, S100A12, S100A13, S100A14, S100A15, S100A16, S100B, S100P, S100Z), chemokine c-c motif (e.g., CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28), insulin-like growth factor (e.g., IGF1, IGF2), galectin (e.g., LGALS1, LGALS2, LGALS3, LGALS4, LGALS7, LGALS8, LGALS9, LGALS10, LGALS12), serum amyloid A (e.g., SAA1, SAA2, SAA3, SAA4).

In one embodiment, the bioactive molecules may be immobilized on membrane through any method known to one skilled in the art including but not limited to EDC/NHS chemistry, maleimide-thiol chemistry, physical adsorption, dip coating, self-polymerization, etc.

In various embodiments, the fluid agents can include one or more additives. In some embodiments, the additives can include vitamins, such as vitamin B2, vitamin Ad, Vitamin D, Vitamin E, and Vitamin K. In various embodiments, the additives can include one or more therapeutics. The therapeutics can be natural or synthetic drugs, including but not limited to: analgesics, anesthetics, antifungals, antibiotics, anti-inflammatoires, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents, or histone proteins, such as histone H4, that destroys cancer cells by lysis upon contact.

Anti-cancer drugs can include chemotherapeutic agents, anti-cell proliferation agents, radiosensitizing agents, or any combinations thereof. For example, any conventional chemotherapeutic agents of the following non-limiting exemplary classes can be screened: alkylating agents; nitrosoureas; antimetabolites; antitumor antibiotics; plant alkyloids; taxanes; hormonal agents; histones; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis(S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. These agents are cell cycle non-specific. Non-limiting examples of antitumor antibiotics include dactinomycin, daunorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action(tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that can also be screened using the bioengineered DWJM.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or combinations thereof. In other aspects, the Bcl-2 family member includes, for example, Bax, Bak, Bcl-Xs, Bad, Bid, Bik, Hrk, Bok, or combinations thereof.

Caspases can include caspase-1, caspase-2, caspase-3, caspase-4, caspase-5, caspase-6, caspase-7, caspase-8, caspase-9, caspase-10, caspase-11, caspase-12, caspase-13, caspase-14, or combinations thereof. In other embodiments, the cytotoxic agent is TNF-«, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or combinations thereof.

Additional anticancer agents include small molecules, peptides, proteins, and synthetic compounds such as: everolimus, trabectedin, abraxane, TLK 286, AV-299, DN⁻¹⁰¹, pazopanib, GSK690693, RTA 744, ON 0910.Na, AZD 6244 (ARRY-142886), AMN⁻¹⁰⁷, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a PD-1 inhibitor, a Cdk inhibitor, an EGFR TK inhibitor, an IGFR-TK inhibitor, an anti-HGF antibody, an anti-CD47 antibody, an anti-GD2 antibody, an anti-EGF receptor antibody, a PI3 kinase inhibitors, an AKT inhibitor, a JAK/STAT inhibitor, immune checkpoint blockades, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolyl-quinolone, vatalanib, AG-013736, AVE-0005, pyro-Glu-His-Trp-Ser-Tyr-D-Ser (But)-Leu-Arg-Pro-Azgly-NH2 x (acetate) wherein X=1 to 2.4, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant,, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and combinations thereof.

In some embodiments, the fluid agents comprise immune cells for immunotherapy. Immunotherapies can include T-cell vaccination, which typically involves immunization with inactivated autoreactive T cells to eliminate a cancer cell population. Another immunotherapy involves the use of a bispecific T-cell Engager (BiTE), which is an antibody designed to simultaneously bind to specific antigens on endogenous T cells and cancer cells as described herein, linking the two types of cells. In certain embodiments, the immunotherapy employs monoclonal antibodies (MAbs). MAbs stimulate an immune response that destroys cancer cells. Similar to the antibodies produced naturally by B cells, these MAbs “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

### Convection-Enhanced Thermo-Chemotherapy Catheter System (CETCS)

The present invention also includes convection-enhanced thermo-chemotherapy catheter systems (CETCS). The CETCS is a remotely operated MRI compatible drug delivery system. The system is configured for treating a variety of diseases and disorders, including but not limited to glioblastomas. The system optimizes distribution of medicines directly into a treatment site by precision positioning of individual microneedles, tissue heating through light delivering fiber optic microneedles, and real-time observation of drug infusion through MRI monitoring. For example, in treating glioblastomas, the system is configured to administer large molecule medicines directly into the cancerous tissue, bypassing the blood brain barrier, and ensuring complete coverage of tumor margins.

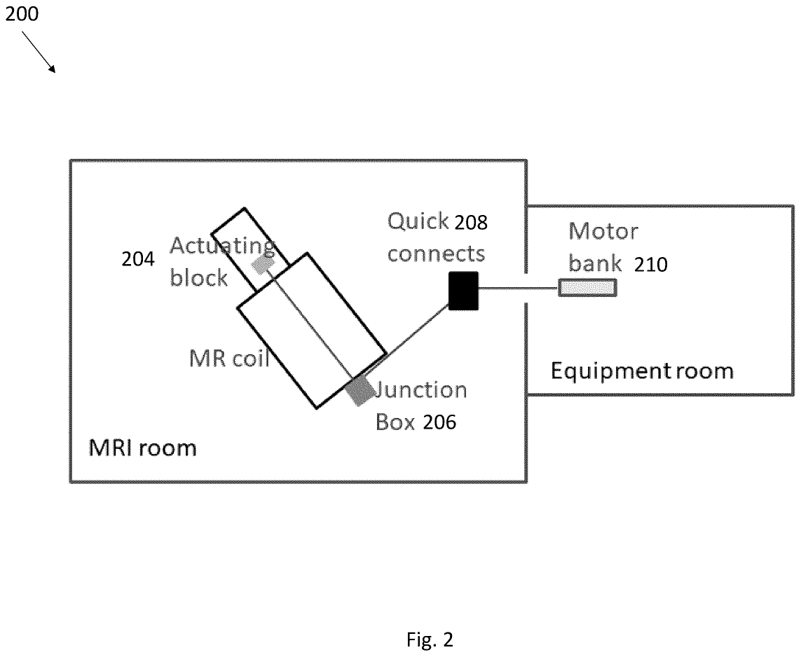

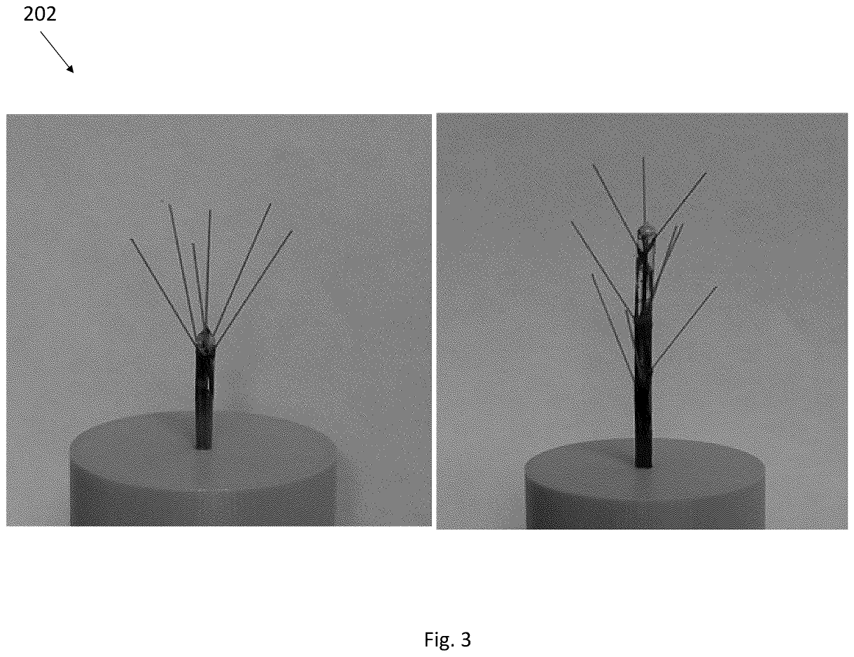

Referring now to FIG. 2, a schematic of an exemplary system 200 is depicted. System 200 comprises a needle assembly 202, an actuating block 204, one or more junction box 206, one or more quick connects 208, and a motor bank 210. An exemplary needle assembly 202 is shown in FIG. 3, wherein a needle assembly 202 comprises a main cannula through which one or more needles are deployable. The left image in FIG. 3 depicts a main cannula configured to deploy six needles (a central needle surrounded by five needles), and the right image in FIG. 3 depicts a main cannula configured to deploy nine needles split between three tiers. It should be understood that needle assembly 202 can comprise any number of needles and is not limited by the arrangements depicted herein. In some embodiments, the needles are fiberoptic needles configured to deliver a fluid agent as well as conduct a laser light for heat application such as those described in U.S. Pat. No. 10,220,124, the contents of which are incorporated by reference herein in its entirety. Mild hyperthermia can increase tissue permeability and thereby increase the dispersal volume of a fluid agent. In some embodiments, internal tubing in the main cannula of needle assembly 202 is used to guide the deployment orientation of the needles.

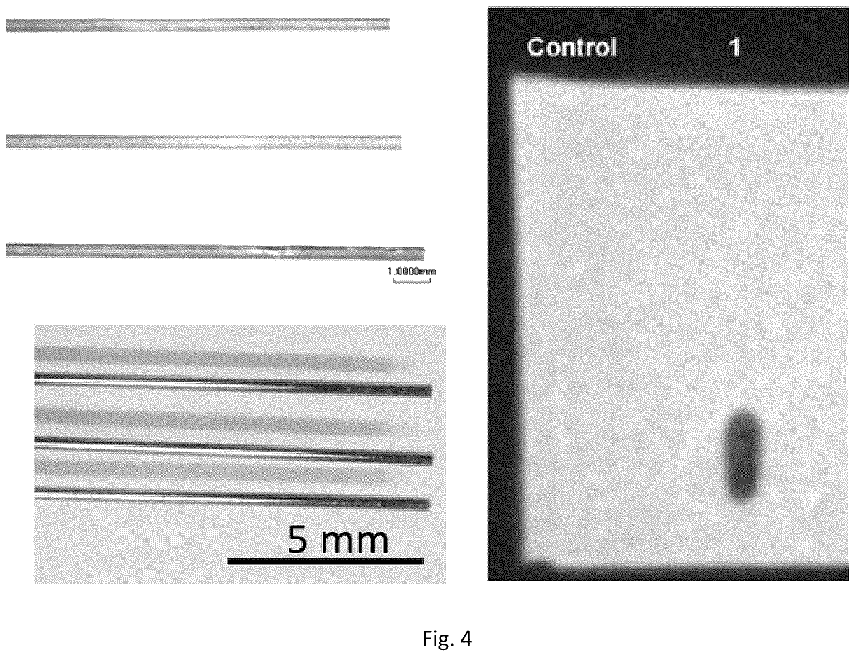

In various embodiments, the needles can be provided with a coating for enhanced visibility, as depicted in FIG. 4. In some embodiments, a needle coating can comprise sections and/or tips of a needle coated with an MR-visible material, including but not limited to gadolinium and titanium. A gadolinium solution can be created by mixing polyimide resin with gadolinium powder then curing the mixture on a needle in an oven. A titanium coating can be applied to a needle for example by first plasma cleaning the surface of the needle and then using e-beam physical vapor deposition to deposit a layer of titanium on the needle.

In various embodiments, needle assembly 202 can further comprise pumps, reservoirs, pressure sensors, and tubing or fluid lines as described elsewhere herein. Tubing or fluid lines fluidly connect reservoirs to the needles of needle assembly 202, and pumps are configured to flow fluid agents to the needles at controllable flow rates. Pressure sensors are provided in the tubing or fluid lines to monitor pressure at the needles, such that system 200 can modulate flow rates to maintain a constant pressure at the needles.

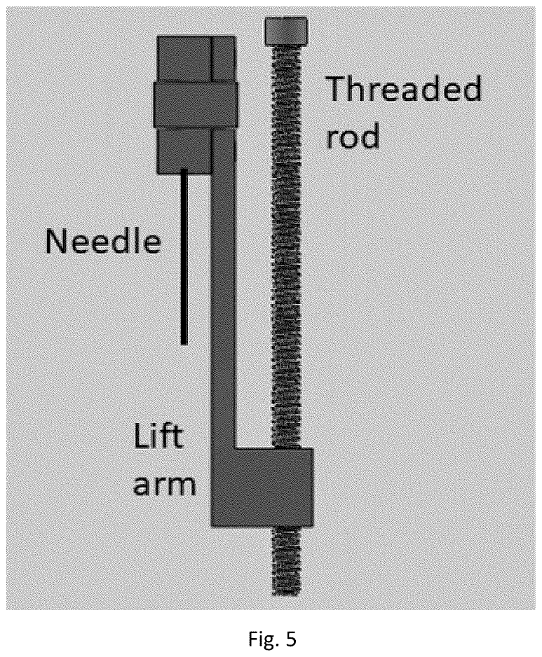

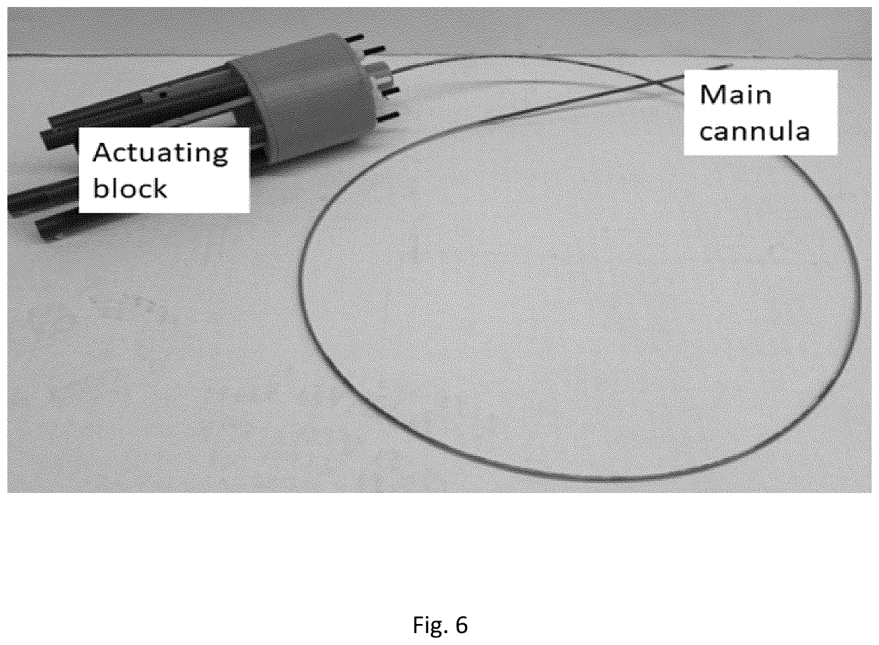

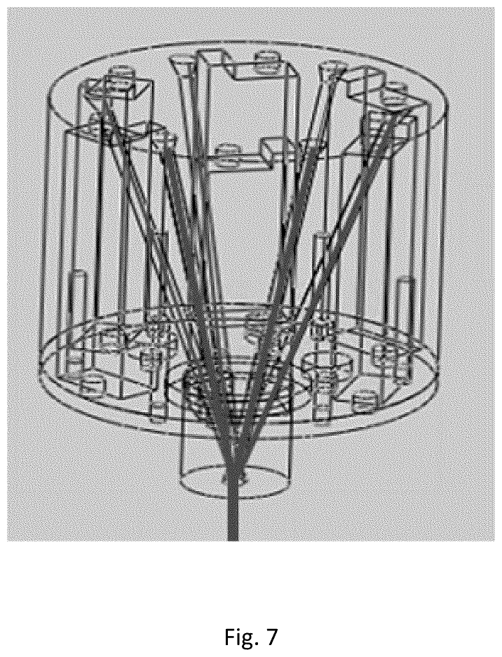

Needles in a needle assembly 202 are actuated using a linear actuator. In some embodiments, a linear actuator or deployment mechanism comprises a lead screw mechanism, as shown in FIG. 5. In certain embodiments, each needle in a needle assembly 202 is individually actuated by a linear actuator powered by a stepper motor. In some embodiments, a plurality of linear actuators may be integrated into an actuating block 204 (FIG. 6) configured to narrow the trajectories of the needles and to force the needles to come together in a flexible tube leading to a rigid main cannula of needle assembly 202 (FIG. 7). The flexible tube permits freedom in positioning actuating block 204 relative to the main cannula of needle assembly 202, allowing the system to be operated in the tight confines of an MRI bore.

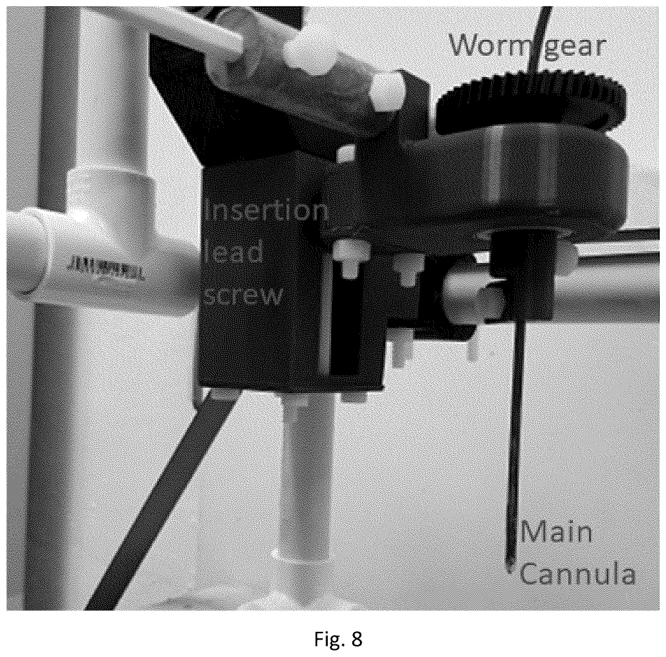

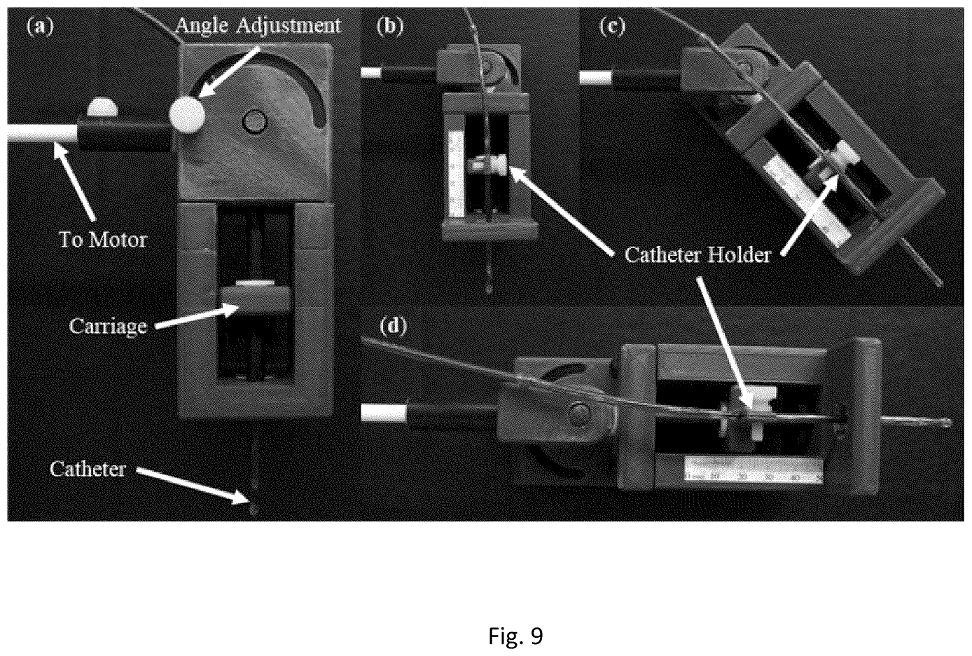

In some embodiments, system 200 further comprises a positioning mechanism for needle assembly 202 configured to orient the main cannula of needle assembly 202 and to move it in proximal and distal directions. An exemplary positioning mechanism is depicted in FIG. 8 comprising a worm gear, wherein the main cannula is held at the center of the worm gear and is rotationally adjustable by the worm gear. The rotational mechanism is mounted on a plate that is operated by a lead screw, allowing insertion and retraction of the main cannula. In some embodiments, a second positioning mechanism is provided, wherein the second positioning mechanism is configured to orient a trajectory of the main cannula of needle assembly 202 (FIG. 9). The second positioning mechanism utilizes two sets of bevel gears that allows a user to set an angle of the main cannula and to deploy the main cannula into a target site, allowing for near infinite insertion trajectories.

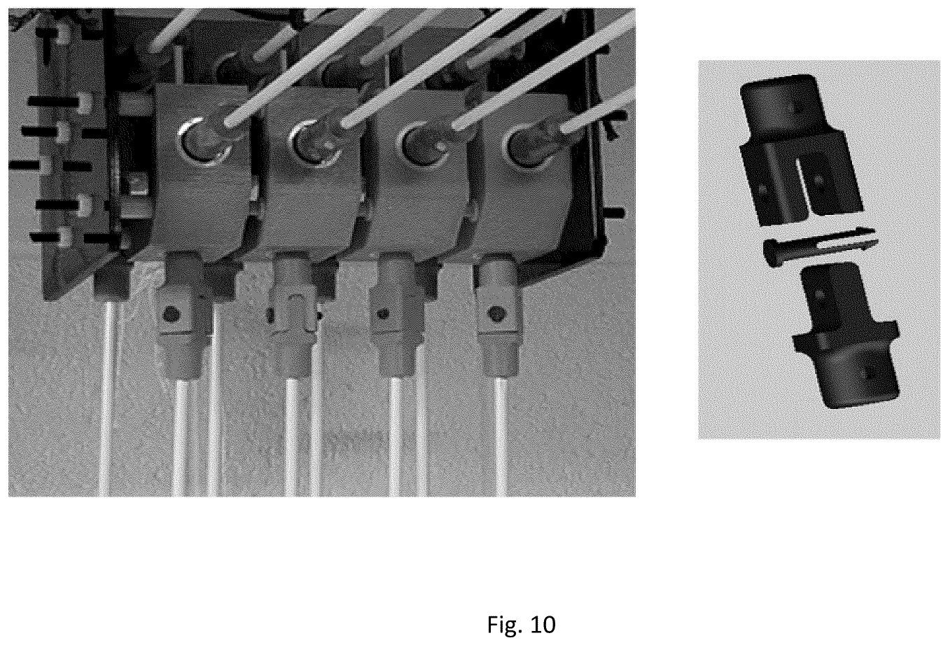

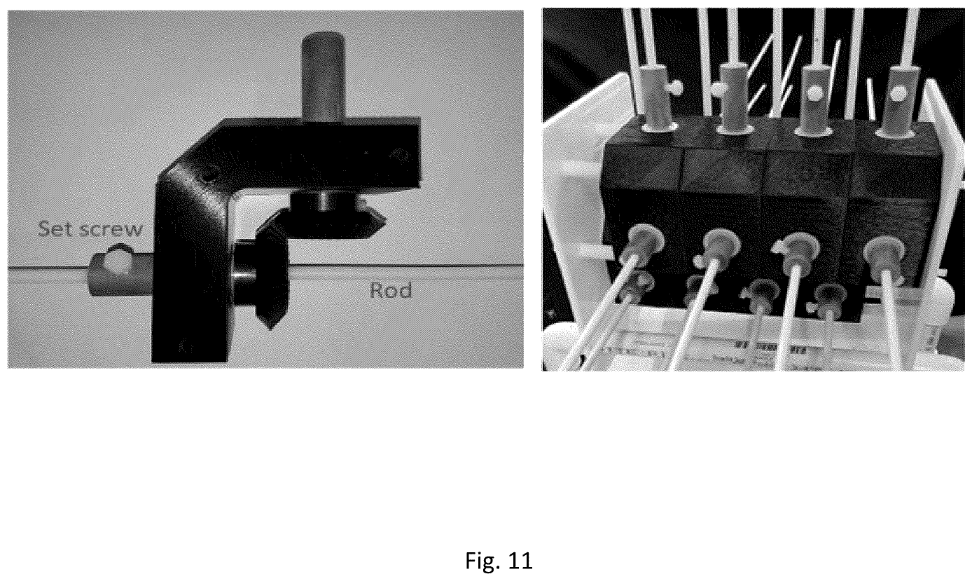

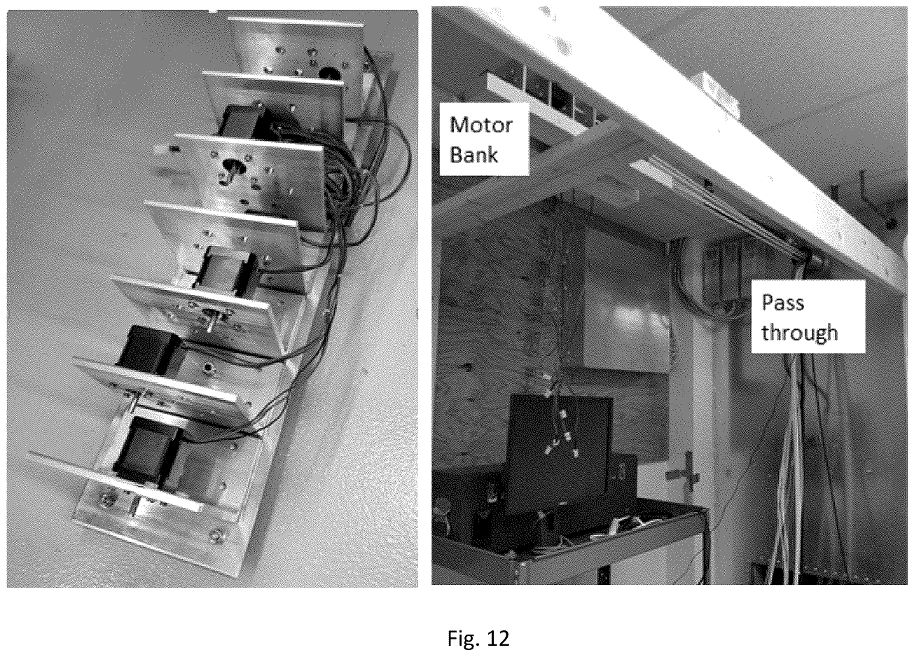

As would be understood by persons having skill in the art, stepper motors and associated wiring may comprise ferromagnetic and/or metallic components, which may be caught by the magnetic field generated by an MRI machine and lead to catastrophic consequences. Accordingly, system 200 implements a transmission system comprising rotating transmission rods between one or more junction box 206 and stepper motors of motor bank 210 (FIG. 12) to transfer positional inputs from the stepper motors to the positioning mechanisms and actuating block 204 (FIG. 13), allowing all ferromagnetic and metallic components to be positioned at a location remote from the magnetic field of an MRI machine. In some embodiments, all components of the transmission system are made entirely from non-metallic components for safe and compatible use in an MRI imaging room. The one or more junction box 206 is configured to allow bends in a transmission path, as shown in FIG. 10 (left). In some embodiments, a junction box 206 comprises plastic universal joints, such as for non-orthogonal bends. In some embodiments, a junction box 206 comprises plastic miter gears for orthogonal bends (visible in FIG. 11, left). In various embodiments, one or more quick connects 208 are used to connect a transmission rod to a junction box 206 (as shown in FIG. 10, right), allowing for the transmission system to be stored out of the way when not in use, wherein each quick connect 208 comprises mated male and female connectors reversibly joined by a clip or tab. The length of the transmission rods can be adjusted to allow for proper positioning of the actuation mechanisms inside of the MRI bore. In some embodiments, the lengths of the transmission rods are adjusted by using hollow shafts in a junction box 206, wherein a transmission rod is tightened into a hollow shaft with a set screw after the actuating mechanisms have been positioned in an MRI coil bore (FIG. 11).

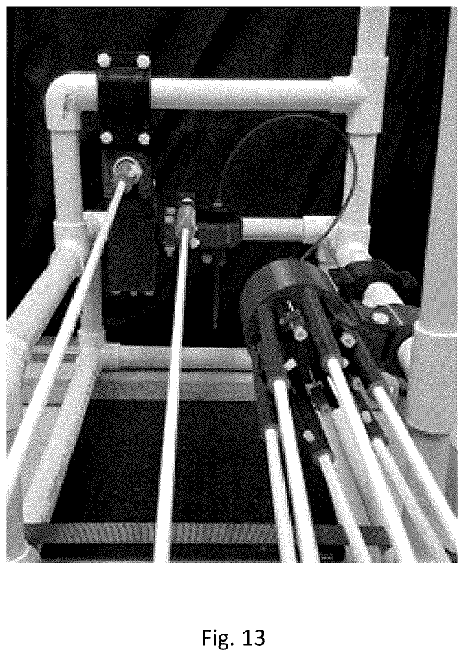

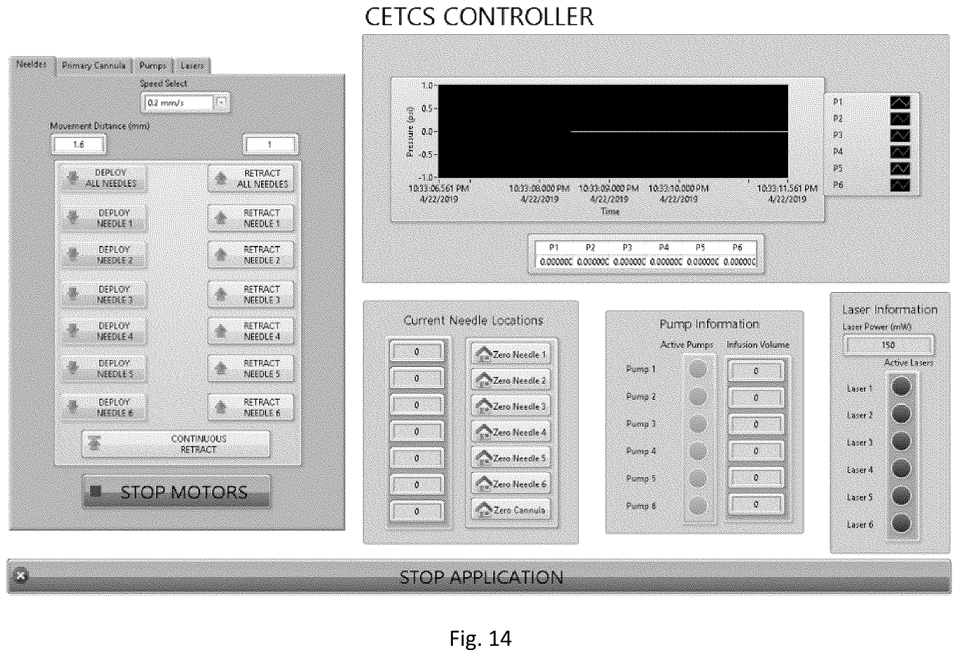

In some embodiments, system 200 further comprises a control system. An exemplary graphical user interface (GUI) for a control system is depicted in FIG. 14, wherein control system enables user control over fluid agent delivery parameters, such that selection among specific patient drug delivery morphologies is possible. Users can use contrast enhanced MR images as well as infusion line pressure readings to determine changes that need to be made to the drug delivery. For example, needles with high pressure readings may indicate needle clogging, such that pumps feeding a clogged needle can be turned off, or that a needle can be repositioned to remove a clog. The control system is also configured to control drug flow rate, laser power, and needle position for each individual needle in a needle assembly 202. In some embodiments, the control system is also configured to control the position of the main cannula for even larger volumes to be delivered.

In some embodiments, the control system comprises a primary graphical user interface configured for user input and treatment modulation, as well as a secondary graphical user interface comprising a proportional-integral-derivative (PID) drug delivery controller configured to control drug delivery pressure. Exemplary control systems suitable for use in a drug delivery controller include, but are not limited to, a proportional-integral-derivative (PID) controller, a proportional-integral (PI) controller, or an adaptive control system, for example which uses external knowledge of the system including mathematical models or large data sets in its control scheme. In some embodiments, multiple control schemes are used in parallel. The drug delivery controller may be configured to continuously monitor infusion pressure and modulate the flow rate of drug infusion to achieve and maintain a target drug delivery pressure. Clinically, convection enhanced delivery (CED) is flow rate driven. However, flow rate gives a user less control over the delivery of a therapeutic as pressure is the primary driver of drug delivery and spread in a tissue. One of the major advantages of infusion with constant pressure is increased control over reflux. Currently, flow rate in addition to catheter geometry is used to prevent reflux. However, the root cause of reflux is the displacement of tissue around a needle, allowing fluid to flow back up a needle tract and out of a target tissue, resulting in low delivery volumes. The displacement of tissue is caused by infusion pressure that exceeds a sealing pressure of the tissue on the needle, therefore a constant pressure drug delivery with infusion pressure under the critical sealing pressure contributes to constant flow rate infusion and avoids reflux.

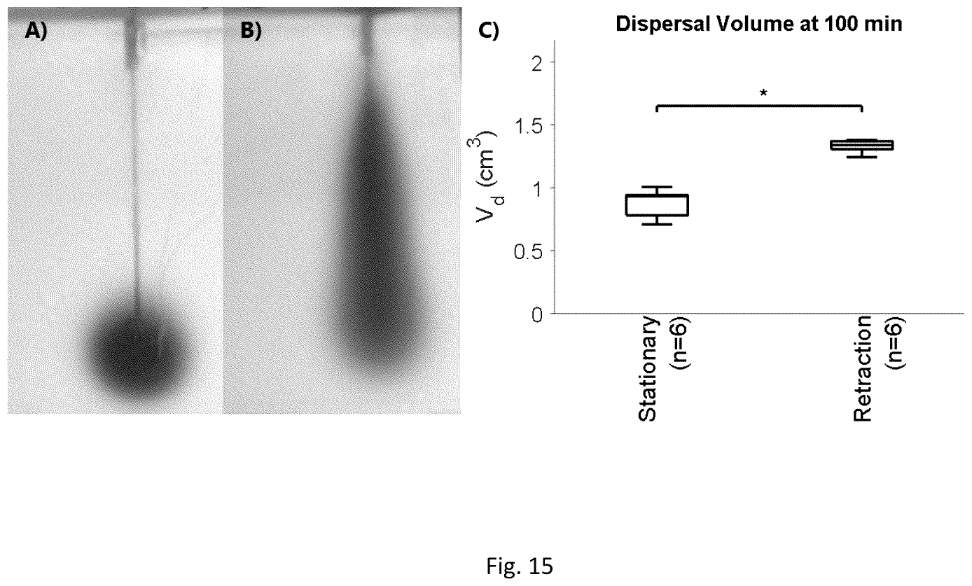

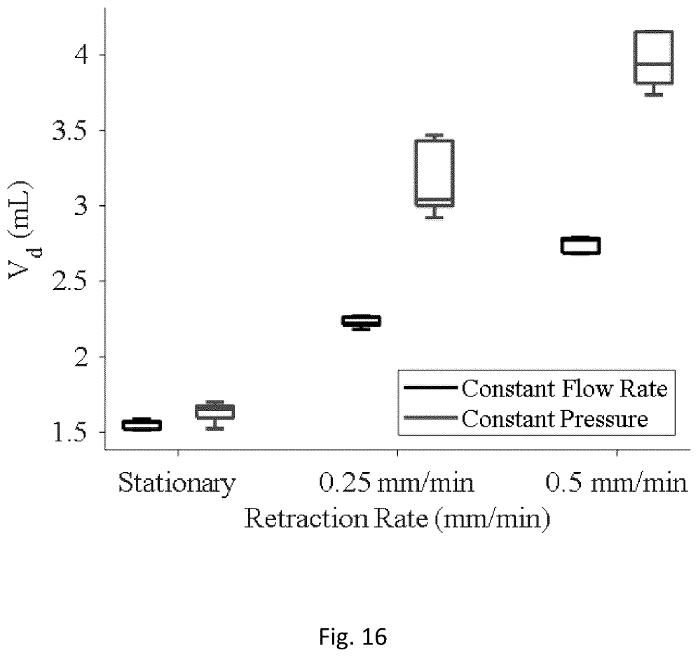

The control system is further configured to provide a continuously and controlled retraction of a needle, which results in a 1.5× increase in dispersal volume of a fluid agent when compared to a standard stationary needle (FIG. 15). The presence of a reflux arresting step change, such as the natural step change between the microneedles and primary cannula in the arborizing catheter adds additional protection against excessive backflow when utilizing continuous needle retraction. Additionally, the use of constant pressure infusions along with controlled catheter movement results in a significantly higher dispersal volume than constant flow rate infusions (FIG. 16).

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Constant Pressure Convection-Enhanced Delivery Increases Volume Dispersed with Catheter Movement in Agarose

Glioblastoma is a World Health Organization (WHO) grade IV tumor and as such is highly aggressive and deadly (Louis DN et al., Acta neuropathologica. 2007 August; 114 (2): 97-109). It is the most common malignant brain tumor representing 14.5% of all brain tumors and 48.6% of all malignant brain tumors (Ostrom QT et al., Neuro-oncology. 2020 Oct; 22 (Supplement_1): iv1-96). Furthermore, glioblastoma has a dismal 5 year survival rate of 7.2%, with median survival of just 8 months 32. The standard recommended treatment according to the National Comprehensive Cancer Network (NCCN) is to resect the tumor to the extent possible, then to administer some combination of radiation therapy, concurrent and/or adjuvant chemotherapy, or supportive care depending on the individual patient's Karnofsky Performance Status (KPS) (Nabors LB et al., Journal of the National Comprehensive Cancer Network. 2020 Nov. 2; 18 (11): 1537-70). Unfortunately, even with aggressive treatments, the tumor is likely to recur near the original tumor site (Hochberg FH et al., Neurology. 1980 Sep. 1; 30 (9): 907; Wallner KE et al., International Journal of Radiation Oncology* Biology* Physics. 1989 Jun. 1; 16 (6): 1405-9), with 72% of patients having recurrence at the 17 month follow up after receiving concurrent radiation therapy and temozolomide (Milano MT et al., International Journal of Radiation Oncology* Biology* Physics. 2010 Nov. 15; 78 (4): 1147-55). A major issue with the use of chemotherapeutic agents is the presence of the blood-brain barrier (BBB) which prevents 98% of small molecules and 100% of large molecules (larger than ~500 Da) from reaching the brain (Pardridge WM, NeuroRx. 2005 January; 2 (1): 3-14). Even with the creation of a leaky BBB caused by many gliomas, the effect is generally focused at the core of the tumor and the BBB stays largely intact especially where infiltrating cells are located resulting in a continued inability to deliver therapeutics to the entire target volume (Van Tellingen O et al., Drug Resistance Updates. 2015 Mar. 1; 19:1-2).

The BBB can be mechanically bypassed through the use of convection-enhanced delivery (CED). CED relies on pressure-driven flow to deliver large payloads of therapeutic agents by implanting a small catheter into the brain parenchyma and delivering therapeutics locally to the region of interest (Bobo RH et al., Proceedings of the National Academy of Sciences. 1994 Mar. 15; 91 (6): 2076-80). CED treatments exhibited great promise in the delivery of therapeutic agents directly to the brain in both preclinical (Bobo RH et al., Proceedings of the National Academy of Sciences. 1994 Mar. 15; 91 (6): 2076-80; Saito R et al., Cancer research. 2004 Oct. 1; 64 (19): 6858-62; Wang W et al., Neurosurgical focus. 2015 Mar. 1; 38 (3):E8) and early clinical trials (Kunwar S et al., Journal of Clinical Oncology. 2007 Mar. 1; 25 (7): 837-44; Laske DW et al., Nature medicine. 1997 December; 3 (12): 1362-8; Lidar Z et al., Journal of neurosurgery. 2004 Mar. 1; 100 (3): 472-9; Rand RW et al., Clinical Cancer Research. 2000 Jun. 1; 6 (6): 2157-65 Weaver M et al., Journal of neuro-oncology. 2003 October; 65 (1): 3-14; Weber FW et al., Local Therapies for Glioma Present Status and Future Developments 2003 (pp. 93-103)). This success led to Phase III clinical trials which had largely disappointing results. The first of which, prompted by the results from Weaver and Laske was stopped prematurely due to low patient response rates (Jahangiri A et al., Journal of neurosurgery. 2017 Jan. 1; 126 (1): 191-200). The second Phase III clinical trial known as the PRECISE trial, and the only one to run to completion to date, failed to show a survival difference between the control arm utilizing the diffusion-based Gliadel wafer and the CED delivery of cintredekin besudotox (Kunwar S et al., Neuro-oncology. 2010 Aug. 1; 12 (8): 871-81). A retrospective study found that a low volume dispersed (Va) of the drug may have contributed to this result, with only 20.1% of the tumor margins covered by the drug (Sampson JH et al., Journal of neurosurgery. 2010 Aug. 1; 113 (2): 301-9).

Methods for increasing V_{d} are numerous, but focus primarily on the fabrication of multiport catheters (Elenes EY et al., Journal of Engineering and Science in Medical Diagnostics and Therapy. 2021 Feb. 1; 4 (1): 011003; Elenes EY et al., Exon Publications. 2017 Sep. 20:359-72; Vogelbaum M A et al., Journal of neurosurgery. 2018 Apr. 13; 130 (2): 476-85) and preventing reflux (Gill T et al., Journal of neuroscience methods. 2013 Sep. 30; 219 (1): 1-9; Krauze MT et al., Journal of neurosurgery. 2005 Nov. 1; 103 (5): 923-9; Vazquez LC et al., Journal of Materials Science: Materials in Medicine. 2012 August; 23 (8): 2037-46; Yin D et al., Journal of neuroscience methods. 2010 Mar. 15; 187 (1): 46-51) which has been shown to be a large issue in the forward distribution of therapeutics with CED to targeted tissue regions (Chen MY et al., Journal of neurosurgery. 1999 Feb. 1; 90 (2): 315-20). It was recently shown that that V_{d} can be increased by 50% in agarose gel brain tissue phantoms using a controlled retracting catheter compared to a standard stationary catheter (Mehta JN et al., Pharmaceutics. 2020 August; 12 (8): 753). This work along with most clinical trials relied on the use of syringe pumps that create a constant flow rate of infusate. One theory that could explain the increase in V_{d} is that controlled retraction catheter creates a larger surface area over which advection can occur by leaving a small void as it moves. If this is the case, an increase in flow rate may be achievable throughout the infusion without causing reflux. In the present work, a controller is developed to allow a syringe pump to be used as a constant pressure source and test the ability of constant pressure infusions coupled with controlled catheter movement to increase Vain agarose gel brain tissue phantoms.

The methods are now described.

### Experimental Setup

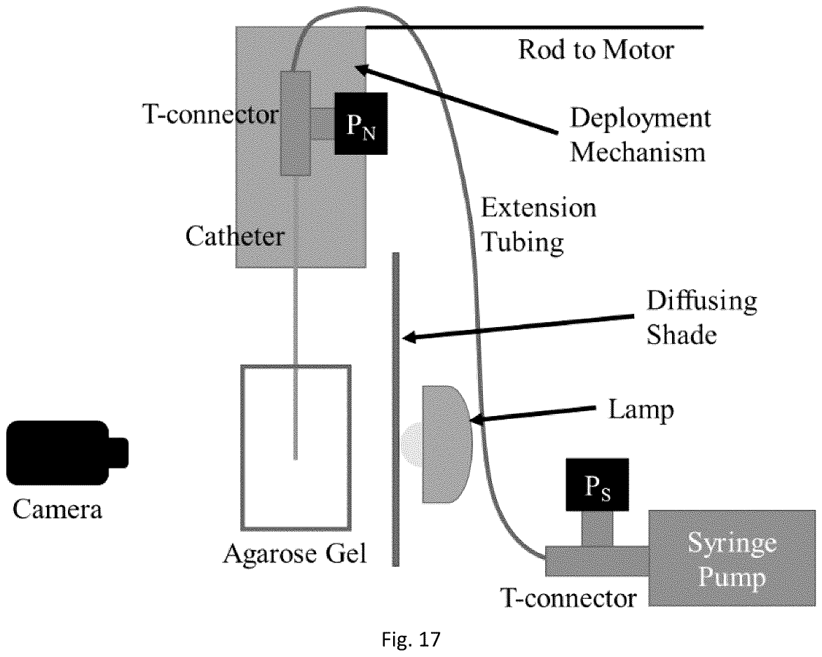

A 23.5 cm long catheter was fabricated by gluing a fused silica capillary tube (150 μm ID/360 μm OD, LTSP150375, Polymicro Technologies, Phoenix, AZ) into a 20 G plastic dispensing needle (920150-PTS, Metcal, Cypress, CA). Approximately 18.5 cm of this catheter was then reinforced with a length of nylon tubing leaving 5 cm of the capillary tubing exposed. As shown in FIG. 17, the catheter was then clamped to a deployment mechanism driven by a leadscrew (¼″-12 with 4 thread starts, 99030A978, McMaster-Carr, Elmhurst, IL). The lead screw was attached to a stepper motor via a set of bevel gears and a fiberglass rod. The stepper motor was controlled by an Arduino programmed to take serial input commands. The catheter was then connected to a t-connector, one end of which was attached to a pressure sensor, P_{N} (26PCBFA6G, Honeywell, golden Valley, MN), the second end was attached to a micro bore extension tubing set (536020, Smiths Medical, Dublin, OH), the proximal end of the tubing set was connected to another t-connector with a second pressure sensor (Ps) on one arm and a 3 mL syringe (309657, Beckton, Dickinson and Company, Franklin Lakes, NJ) on the third arm. Finally, the syringe was inserted into a to a syringe pump (Fusion 100, Chemyx, Stafford, TX) which was programmed to create the desired flow rate.

### System Model

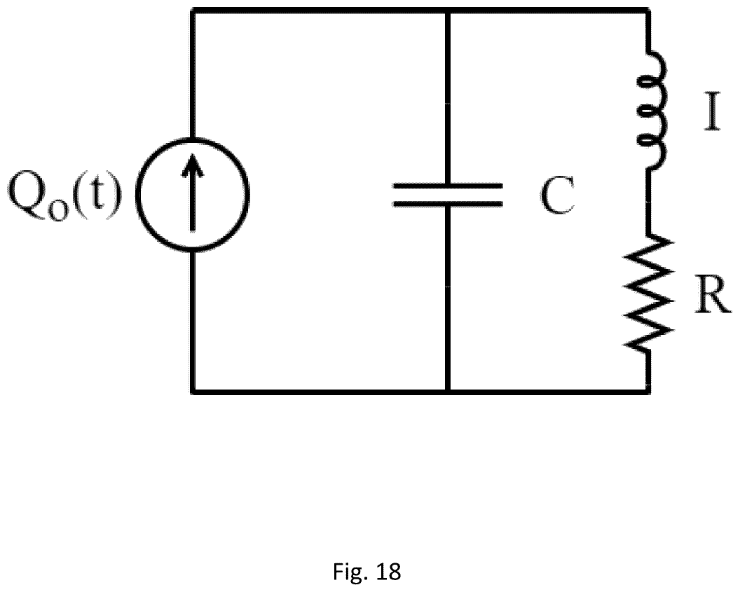

The system dynamics of a catheter infusing into an agarose gel brain tissue phantom can be modeled as unsteady Poiseuille flow through a cylinder. In the experimental setup detailed in the following section, a syringe pump was used to create a constant flow rate infusion through a 3 mL syringe (309657, Beckton, Dickinson and Company, Franklin Lakes, NJ), connected to a micro bore extension tubing set (536020, Smiths Medical, Dublin, OH), and finally through a custom catheter (Mehta JN et al., Frontiers in Biomedical Devices 2021 Apr. 12 (Vol. 84812, p. V001T12A012)), as shown in FIG. 17. Additionally, pressure sensors are connected through t-connectors at the inlet (Ps) and exit (P_{N}) of the extension tubing. This system can be viewed as a simple pipe network containing inductance, resistance, and capacitance. In this analogy, the inductance and resistance for the extension tubing and the 3-way t-connector was considered negligible given the small bore diameter of the catheter (150 μm) compared to both the extension tubing diameter (500 μm) and the 3 way t-connector (500 μm). The hydraulic circuit for this system is shown in FIG. 18 and is analogous to an RLC electronic circuit. The capacitance represents the change in volume of extension tubing that may happen when a large pressure change occurs. It should be noted that there is some disagreement regarding the best approach to modeling lumped-parameter fluid flow. Conventionally the capacitance of the system is treated as an element in series with the resistive and inertia elements which leads to an asymmetry because the volume storage is confined to the capacitor when in reality the capacitor, resistor, and inertia are all the same component in the system. Previous literature suggests placing the capacitor in parallel with the inertia and resistor elements. This also leads to an issue since in placing the capacitor in parallel, the model does not properly capture the flow rate into and out of the line. This configuration, using a capacitor in parallel, results in a dynamic system with two states which is summarized in Eq. 1:

$$[ \begin{matrix}{\dot{Γ}}_{I} \\ {\dot{V}}_{C}\end{matrix} ] [ \begin{matrix}- \frac{R}{I} & \frac{1}{C} \\ \frac{1}{I} & 0\end{matrix} ] [ \begin{matrix}{Γ}_{I} \\ {V}_{C}\end{matrix} ] + [ \begin{matrix}0 \\ 1\end{matrix} ] {Q}_{0} ( t ) \qquad ( Eq . 1 )$$

$${P}_{N} = [ 0 \frac{1}{C} ] [ \begin{matrix}{Γ}_{I} \\ {V}_{C}\end{matrix} ]$$

where Γₗ is the fluid momentum through the inertia element, V_{C} is the volume of the extension tubing, Γₗ is the pressure across the inertia element, \dot{V}_{C} is the volumetric flow rate through the extension tubing, and P_{N} is the pressure going into the needle and is the location of the pressure sensor. The resistance, R, can be estimated using Poiseuille's law for steady flow through a cylinder, shown in Eq. 2:

$$R = \frac{128 μ L}{π {D}^{4}} \qquad ( Eq . 2 )$$

where μ is the dynamic viscosity of the fluid, L is the length of the line, and D is the internal diameter of the line. The fluid inertance, I, can be calculated from Eq. 3:

$$I = \frac{ρ L}{A} \qquad ( Eq . 3 )$$

where ρ is the density of the fluid and A is the cross-sectional area of the line. Finally, the capacitance C can be found by relating the hoop stress in a tube and Hook's Law and simplifies to Eq. 4:

$$C = \frac{{V}_{0} {d}_{i}}{Et} \qquad ( Eq . 4 )$$

where V₀ is the initial volume of the compliant tube, dᵢ is the internal diameter of the elastic tube, E is the elastic modulus of the elastic tube, and t is the wall thickness of the tube. Parameters for R, I, and C were used as an initial guess for a gray box system model of the same form shown in Eq. 1. The R, I, and C values found from Eq. 2 through Eq. 4 were not used directly to create the final system model because the properties of the agarose gel tissue phantom which the catheter infused into are not known. The final parameters of a grey box model were determined using MATLAB's system identification toolbox (R2018a, MathWorks, Natick, MA). The data used for the system identification were the stationary constant flow rate infusions, described in the experimental setup section. While data for evaluation of V_{d} for these constant flow rate infusions were gathered for 100 minutes, the infusions were actually run for 101 minutes at 1 μL/min and then an additional 15 minutes of infusion occurred at 0.5 μL/min. The addition of 1 minute at 1 μL/min was implemented to ensure that there was a sufficient gap between the main data set and the lower flow rate data.

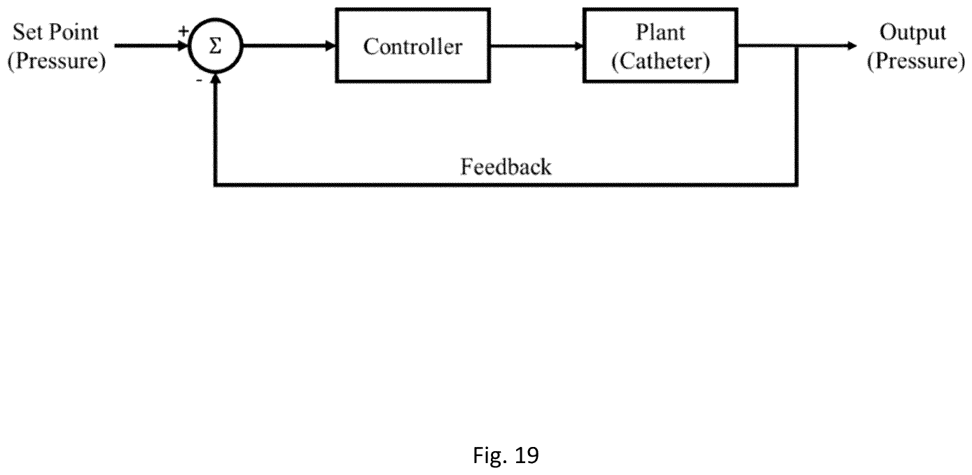

The identified parameters were used in order to tune a PID controller. The equation representing the PID algorithm is shown in standard form in Eq. 5:

$$u ( t ) = {K}_{P} ( e ( t ) + \frac{1}{{T}_{i}} {\int }_{0}^{t} e ( t ) d t + {T}_{d} \frac{de ( t )}{dt} ) \qquad ( Eq . 5 )$$

where u(t) is the syringe pump flow rate, e(t) is the error between the target pressure and the actual pressure, K_{P} is the proportional gain constant, Tᵢ is the integral time constant, and T_{d} is the derivative time constant. The PID tuning parameters, K_{P}, Tᵢ, and T_{d} can be found using a PID tuning algorithm in MATLAB (R2018a, MathWorks, Natick, MA) in conjunction with the estimated system model. The PID tuning algorithm's design focus was on reference tracking to minimize overshoot as overshoot in pressure may increase the likelihood of reflux. With this design, the chosen tuning parameters were as follows: K_{P}=4.88E-7, Tᵢ=1.91 min, and T_{d}=1.12E-4 min, which results in a modeled overshoot of 0.32%, a rise time of 2.40 min and a settling time of 3.91 min. This controller was implemented in a custom Lab View program (National Instruments, Austin, TX) which measured the infusion line pressure and controlled the flow rate to the syringe pump. A block diagram of the controller is shown in FIG. 19.

### Agarose Infusions

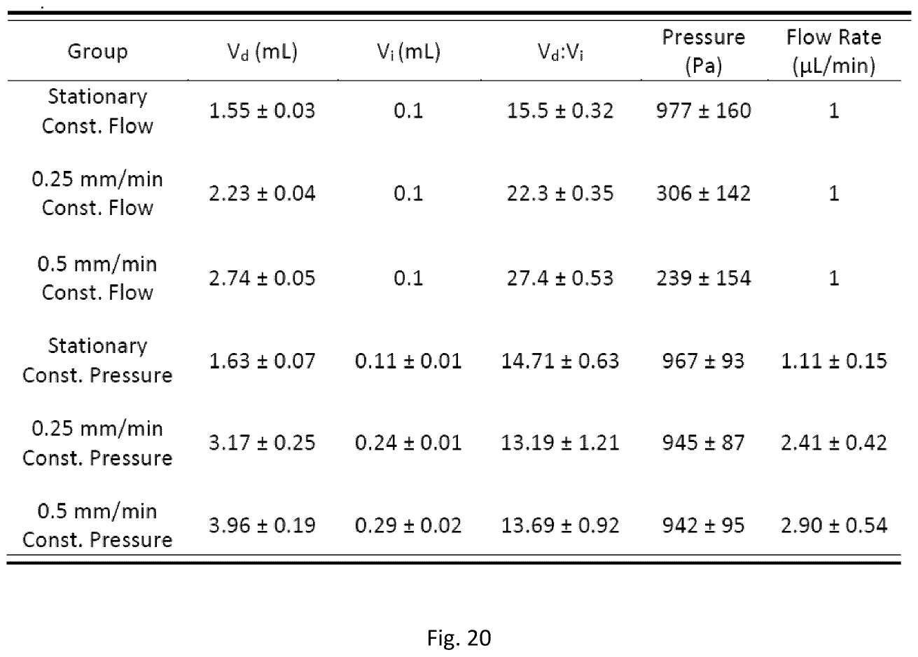

In each experiment, the catheter was manually guided through a small hole in the lid of the agarose gel phantom just above the surface of the gel. Then, using the automated deployment mechanism, the catheter was inserted 32 mm into the 0.6% (wt./wt.) agarose gel brain tissue phantom, at a rate of approximately 13 mm/s, and then retracted by 2 mm at the same rate. Retraction has been shown by other groups to mitigate clogging/coring that may have occurred during insertion (Martanto W et al., 2006 May 30; 112 (3): 357-61). Infusion of 1% (wt./wt.) indigo carmine dye was then infused through the catheter for the duration of the experiment. Six experimental groups were used as shown in FIG. 20:1) Stationary (0 mm/min), constant flow; 2) retracting (0.25 mm/min), constant flow; 3) retracting (0.5 mm/min), constant flow; 4) stationary (0 mm/min), constant pressure; 5) retracting (0.25 mm/min), constant pressure; and 6) retracting (0.5 mm/min), constant pressure. The constant flow rate was 1 μL/min, consistent with previous work (Elenes EY et al., Exon Publications. 2017 Sep. 20:359-72; Hood R L et al., Optical Fibers and Sensors for Medical Diagnostics and Treatment Applications XIII 2013 Mar. 20 (Vol. 8576, p. 85760G); Mehta JN et al., Pharmaceutics. 2020 August; 12 (8): 753) and the constant pressure target value was chosen to be the mean pressure of the stationary constant flow infusions (977 Pa). The total infusion time was 100 minutes. The maximum retraction distance was 25 mm, therefore the 0.25 mm/min retracting catheter moved continuously throughout the experiment, but the 0.5 mm/min retracting catheter moved continuously for the first 50 minutes and then stayed stationary for an additional 50 minutes. Pressure from both P_{N} and Ps were recorded once per second; however, only pressure from the P_{N} sensor was used for analysis due to the relative motion between the catheter and the Ps sensor producing variable pressure readings. Optical images were acquired once a minute using a digital camera with a CMOS sensor (Rebel Tli, Cannon Inc., Ohta-ku, Tokyo, Japan). Images were then post processed using Matlab as detailed previously (Elenes EY et al., Exon Publications. 2017 Sep. 20:359-72; Mehta J N et al., Pharmaceutics. 2020 August; 12 (8): 753). It should be noted that the red intensity channel was used to convert the images from RGB to grayscale, similar to what was done in previous studies (Elenes EY et al., Exon Publications. 2017 Sep. 20:359-72) but divergent from the method used in a recent study (Mehta JN et al., Pharmaceutics. 2020 August; 12 (8): 753) where the “RGB2GRAY” algorithm was used in Matlab. The use of the red intensity channel was found to create more contrast between the infusion cloud and surrounding background while also creating less contrast between the catheter and the surrounding background. Similar to the previous experiment (Mehta JN et al., Pharmaceutics. 2020 August; 12 (8): 753), a threshold for converting grayscale images to binary was found by measuring the grayscale intensity of indigo dye concentrations in agarose gel of 5 concentrations: 0, 0.01, 0.05, 0.1, and 0.5% of the stock concentration. Concentration standards were created at the beginning of each day and were used to create the imaging threshold on a day-by-day basis allowing for small variations in the dye concentration and ambient lighting to be properly accounted for. A threshold of 0.05% of the stock concentration was found to reliably distinguish between the infusion cloud and the background and was therefore used as the threshold value for all experiments. A total of five successful infusions in each group were obtained. Exclusion criteria for successful constant flow rate infusions were pressure deviation of more than 1.5 times the mean standard deviation from the time varying mean pressure of all included infusions for longer than 15% of the infusion time. The constant pressure infusions resulted in much tighter pressure windows, often within the repeatability of the pressure sensors. Therefore, results were discarded if the pressure strayed from the set point by more than the sensor repeatability for over 15% of the infusion time. A one-way analysis of variance was conducted to test for significant differences in both V_{d} and in infusion line pressure at the needle (P_{N}). The analysis was followed by a post-hoc Tukey's test. The results are now described.

FIG. 20 shows the results from the five infusions for each group. V_{d} for each group was significantly different from other groups (p<0.001) with the exception of the two stationary catheters which were statistically the same (p=0.93). The two retracting constant pressure catheters resulted in significantly higher Vᵢ and flow rates than all other groups (p<0.001) and the stationary constant pressure catheter resulted in higher Vᵢ and flow rates than the constant flow rate groups (p=0.038). However, significant increases in mean distribution ratios (V_{d}:Vᵢ) only occur with the retracting constant flow rate catheters which have significantly higher ratios than all other groups (p<0.001). While the continuously retracting constant pressure catheters have significantly lower mean distribution ratios than all constant flow rate groups (p<0.01), and the stationary constant pressure catheter has statistically similar ratios to the stationary constant flow rate catheter (p=0.51). The stationary constant pressure catheter had significantly higher mean distribution ratio than the 0.25 mm/min constant pressure catheter (p=0.03), and no statistical difference in mean distribution ratio was found between the two retracting constant pressure catheters (p>0.2). Finally, the retracting constant flow rate groups resulted in significantly lower mean infusion pressures compared to all other groups (p<0.001), but the mean infusion pressure between the two groups were statistically similar (p=0.55). Infusion pressures between the stationary constant flow rate, stationary constant pressure, and retracting constant pressure groups were statistically the same (p>0.95).

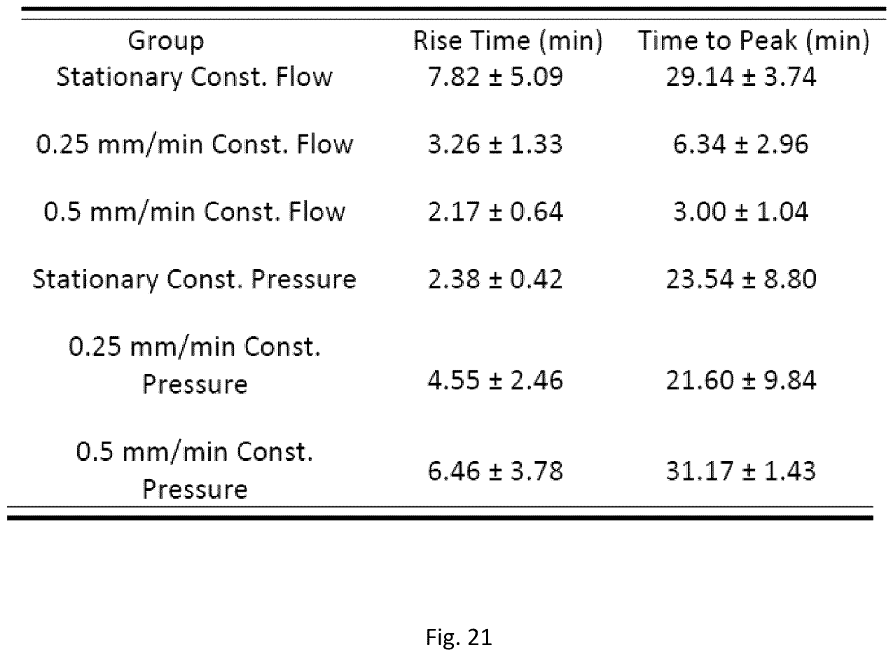

FIG. 21 shows the rise time and time to maximum pressure for each group. Rise time was calculated as the time it took for pressure to rise from 10% to 90% of the peak pressure reading. While time to peak pressure was defined as the time it took for pressure to rise to its highest value. With the exception of the stationary catheter, the constant pressure infusions took longer to rise in pressure and to reach the maximum pressure.

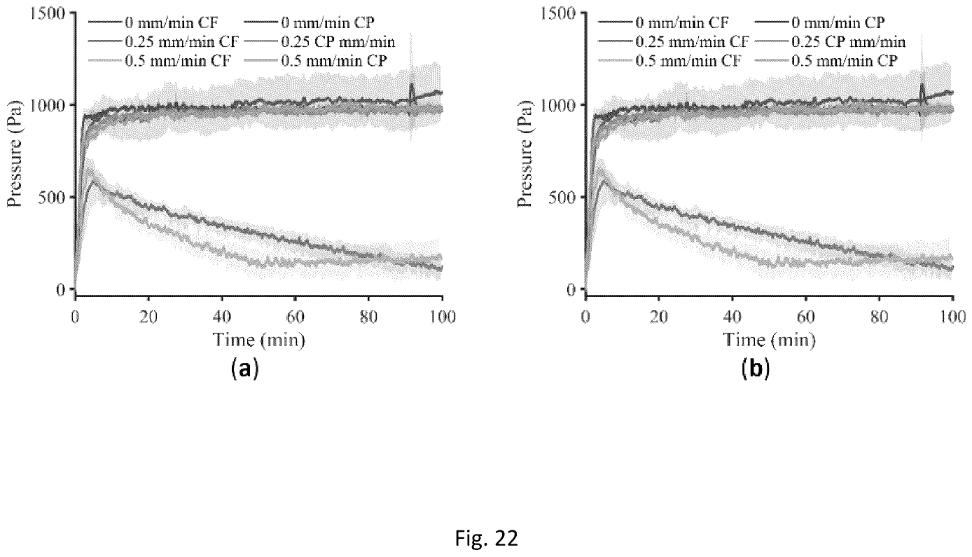

FIG. 22(a) shows the time varying mean of pressure for each catheter group and FIG. 22 (b) shows the time varying flow rate for each catheter group. As seen in FIG. 22(a), the constant flow rate retracting catheters result in a decrease in pressure over time and the retracting constant pressure catheters result in steady pressure measurements throughout the infusion. The 0.5 mm/min catheter reaches an equilibrium point at 50 minutes into the infusion due to catheter retraction ceasing at 50 minutes, whereas pressure continues to drop throughout the entire infusion with the 0.25 mm/min catheter because it continues to move until the end of the infusion. FIG. 22(b) shows the opposite trend, with the constant flow rate catheters showing steady infusion flow rate throughout the infusion and an increase in flow rate with the retracting constant pressure catheters. Again, the 0.5 mm/min constant pressure catheter reaches an equilibrium flow rate at the 50 minute time point and the 0.25 mm/min constant pressure catheter continues to increase in flow rate throughout the infusion.

The use of variable flow rate infusions with controlled catheter retraction appears to increase the overall V_{d} of infusions with a 105% increase and a 155% increase in V_{d} found between the 0.25 mm/min and 0.5 mm/min constant pressure catheters relative to the stationary constant flow catheter, respectively. This is likely due to the increase in surface area that is caused by the catheter movement and small void left by the catheter. The clear tradeoff is an increase in Vᵢ as the flow rate increases with retraction distance. This increase in Vᵢ also results in a reduction in the mean distribution ratio which becomes lower than that of the stationary constant flow rate catheter. This result is expected as the flow rate is increasing to maintain constant pressure. If pressure is the primary driving force for fluid transport, over diffusion as previously suggested (Andriani RT, Design and validation of medical devices for photothermally augmented treatments (Doctoral dissertation, Virginia Tech); Raghavan R et al., Neurosurgical focus. 2006 Apr. 1; 20 (4):E12), then it follows that V_{d} would scale proportionally to Vᵢ, as indicated by the mean distribution ratios of the constant pressure infusions.

The increase in V_{d} between the constant flow rate stationary and 0.25 mm/min retracting catheters was about 44% which is lower than, but similar to that previously reported (Mehta J N et al., Pharmaceutics. 2020 August; 12 (8): 753), this slight discrepancy may be due to the chosen thresholding concentration differences between the two studies. The stationary catheters result in a smaller infusion volume which would indicate higher concentrations of the infusate; whereas the controlled retraction catheter results in a larger volume with lower concentrations, especially on the periphery of the volume This highlights the importance of determining minimum therapeutic drug concentrations as large spatial variations in concentration may occur especially as the mean distribution ratio increases.

It appears that increased retraction rates, regardless of the infusion type (constant flow rate or constant pressure) results in a marked increase in V_{d}, with the 0.5 mm/min constant pressure catheter resulting in an increase in V_{d} of about 25% compared to the 0.25 mm/min constant pressure catheter. Similarly, the 0.5 mm/min constant flow rate catheter exhibited a 23% increase in V_{d} compared to the 0.25 mm/min constant flow rate catheter. However, the largest increase in V_{d} between successive retraction rates was found between the stationary and 0.25 mm/min catheters with a 94% increase in V_{d} found for the constant pressure catheters and a 44% increase for the constant flow rate catheters. This slowing in V_{d} growth with rate may be attributable a smaller concentration of dye on the periphery of the infusion clouds of the fastest moving catheters that could not be reliably measured with the described imaging techniques. However, it is also possible that there is diminishing return in V_{d} with increased catheter movement rate due to the maximum retraction distance of 25 mm resulting in the retraction to stop halfway through the infusion with the quickest moving catheter. Finally, the reduction in Va growth could be due to some level of infusion cavity collapse after the catheter has moved.

The rise time of the stationary constant pressure catheter is within 1% of the rise time predicted by the system model when using the PID controller. However, the rise times for the 0.25 mm/min and 0.5 mm/min retracting constant pressure catheters are 62% and 92% slower than that predicted by the model, respectively. This is likely due to the catheter movement which serves as a disturbance to the system. Since the system is constantly being disturbed, the pressure does not respond as quickly. This is accentuated by the decision to minimize overshoot which necessarily results in a controller that is slower to respond to disturbances in the system. However, the mean pressure for the two constant pressure retracting groups is maintained to within 4% of the target pressure which is well within the repeatability of the sensors used. The stationary constant pressure catheter performs even better maintaining a pressure within 1% of the target pressure.

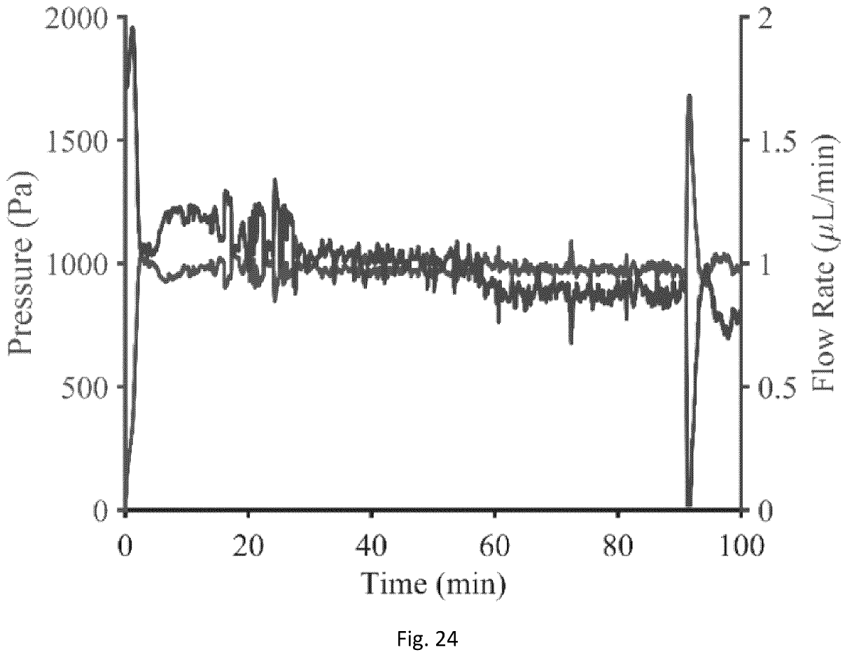

The plots of pressure and flow rate shown in FIG. 22 for the stationary constant pressure catheter show a spike in pressure corresponding to a dip in flow rate, this disturbance was caused by one infusion, the time varying pressure and flow rate of which are shown in FIG. 24. The spike shown at about 91 minutes into the infusion might be the result of an undissolved particulate of indigo carmine dye entering the catheter and then being expelled in the time span of about 2 minutes. This pressure spike did not appear to be correlated to the creation of any reflux or any other artifacts.

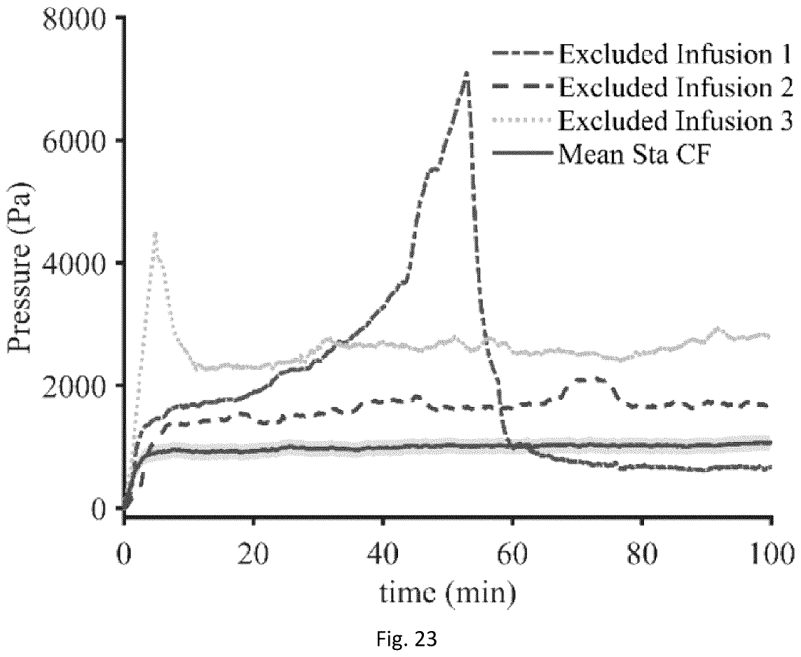

The time varying pressure shown in FIG. 23 are from three stationary constant flow rate infusions that were removed due to the pressure exclusion criteria. Excluded Infusion 3 had an immediate increase in pressure with its peak at about 5 minutes at which point the pressure dropped rapidly and stayed steady, although significantly higher than the successful infusions (purple line) for the remainder of the infusion. The pressure in Excluded Infusion 3 appears to climb up to the 53 minute time marker at which point the pressure dropped rapidly to below that of the successful infusions. Both Excluded Infusion 1 and 3 likely became clogged during insertion, this clog appears to be at least partially expelled, resulting in the quick drop in pressure. No visible reflux occurred for any of the excluded infusions. Even after expulsion Excluded Infusion 3 had a significantly higher mean pressure than the successful infusions, a similar trend is seen Excluded Infusion 2. This likely indicates a partial occlusion of the catheter tip, enough to raise the steady state pressure, but not enough to cause the pressure to continue to rise and eventually reach a pressure high enough to fully expel the clog. These results seem to corroborate those presented by Lam et al., where pressure rise does not occur instantaneously if a catheter become clogged rather it happens over time (Lam MF et al., Journal of neuroscience methods. 2014 Jan. 15; 221:127-31). While this may not be useful in quick infusions such as those conducted by Lam et al., in longer infusions such as the ones presented here and commonly occurring clinically (Vandergrift WA et al., Neurosurgical focus. 2006 Apr. 1; 20 (4):E13), the difference in pressure appears clear when a catheter is clogged and therefore may be utilized as one indicator of a successful infusion.

Reflux, while not formally quantified in this study, was not observed during any infusion. This finding is consistent with the theory that tissue deformation is the critical factor in creating reflux (Lueshen E et al., Medical engineering & physics. 2017 Jul. 1; 45:15-24; Morrison P F et al., American Journal of Physiology-Regulatory, Integrative and Comparative Physiology. 1999 Oct. 1; 277 (4): R1218-29; Orozco GA et al., Medical & biological engineering & computing. 2014 October; 52 (10): 841-9; Raghavan R et al., Physics in Medicine & Biology. 2009 Dec. 11; 55 (1): 281) since all infusion line pressures were at or below that of the stationary constant 1 μL/min catheter, a flow rate known to not create reflux in agarose. Furthermore, since it has been shown that reflux can be mitigated if it is found early enough and corrected for by reducing infusion flow rate (Lewis O et al., Journal of neuroscience methods. 2018 Oct. 1; 308:337-45), there may be an additional benefit of utilizing constant pressure infusions, as the pressure can be slowly ramped until a small amount of reflux is observed, then pressure can be reduced such that it is just below the critical reflux inducing pressure. This would ensure that the maximum flow rate is obtained while minimizing or preventing reflux.

Finally, the limitations associated with the use of agarose gel should be noted. Agarose gel brain tissue phantoms are commonly used in benchtop setups to evaluate CED treatment technologies (Elenes EY et al., Exon Publications. 2017 Sep. 20:359-72; Gill T et al., Journal of neuroscience methods. 2013 Sep. 30; 219 (1): 1-9; Hood RL et al., Optical Fibers and Sensors for Medical Diagnostics and Treatment Applications XIII 2013 Mar. 20 (Vol. 8576, p. 85760G); Krauze MT et al., Journal of neurosurgery. 2005 Nov. 1; 103 (5): 923-9; Lam MF et al., Journal of neuroscience methods. 2014 Jan. 15; 221:127-31; Lewis O et al., Journal of neuroscience methods. 2018 Oct. 1; 308:337-45; Mehta J N et al., Pharmaceutics. 2020 August; 12 (8): 753; Sillay K et al., Stereotactic and functional neurosurgery. 2013; 91 (3): 153-61) and have been suggested to be a viable surrogate for the brain's interstitial space because of its porolastic nature (Chen ZJ et al., IEEE Transactions on Biomedical Engineering. 2002 Aug. 7; 49 (2): 85-96; Chen ZJ et al., Journal of neurosurgery. 2004 Aug. 1; 101 (2): 314-22; Raghavan R et al., Physics in Medicine & Biology. 2009 Dec. 11; 55 (1): 281) and similar pore size (Gillies GT et al., Nanotechnology. 2002 Jun. 21; 13 (4): 484). However, agarose gel is isotropic, and cannot model the heterogeneity of real tissue due to structures such as cells, blood vessels, and ventricles, pathways in which infusions are known to follow (Raghavan R et al., Physics in Medicine & Biology. 2009 Dec. 11; 55 (1): 281). Finally, as suggested by Casanova et al. (Casanova F et al., PLOS One. 2014 Apr. 28; 9 (4): e94919; Casanova F et al., J Biomech Eng. 2012 April 134 (4): 41006), catheter manipulation may result in a different response in agarose gel than in the brain (Sillay K et al., Journal of neural engineering. 2012 Feb. 14; 9 (2): 026009).

### Example 2: Convection-Enhanced Delivery with Controlled Catheter Movement: A Parametric Finite Element Analysis

Glioblastoma (GBM) is the most common malignant brain tumor and comprises about 48.3% of all malignant tumors (Ostrom QT et al., Neuro-oncology. 2019 Oct; 21 (Supplement_5): v1-00). GBM is highly infiltrative, mitotically active, and necrosis-prone resulting in its World Health Organization (WHO) Grade IV designation (Louis DN et al., Acta neuropathologica. 2007 August; 114 (2): 97-109). Current standard of care therapy includes maximal possible resection followed with radiation therapy and concurrent and/or adjuvant chemotherapy (Nabors LB et al., Journal of the National Comprehensive Cancer Network. 2020 Nov. 2; 18 (11): 1537-70). Despite the use of aggressive treatment strategies, tumor recurrence is unavoidable and generally presents at or near the original tumor site (Wallner KE et al., Int J Radiat Oncol Biol Phys. 1989 June; 16 (6): 1405-9; Hochberg FH et al., Neurology. 1980 Sep. 1; 30 (9): 907). This results in a median survival of only 12 to 15 months (Wen PY et al., New England Journal of Medicine. 2008 Jul. 31; 359 (5): 492-507) and a low 5 year survival of 6.8% (Ostrom QT et al., Neuro-oncology. 2019 October; 21 (Supplement_5): v1-00).

Treatment of GBM is severely complicated by the infiltrative nature of the tumor, resulting in the presence of tumorous cells centimeters beyond the distinct visible boundaries shown on Magnetic Resonance (MR) scans (DeAngelis L M, New England journal of medicine. 2001 Jan. 11; 344 (2): 114-23), making complete resection of the tumor impossible. Additionally, the presence of the blood-brain barrier (BBB) and blood-brain-tumor barrier (BBTB) inhibit the systemic delivery of therapeutic agents. The BBB prevents the transport of 98% of small molecule drugs with a molecular mass of under 400 to 500 Da (Pardridge WM, NeuroRx. 2005 January; 2 (1): 3-14). While high-grade gliomas, like GBM, disrupt the BBB, forming a leaky BBTB, the local disturbance of the BBB is unlikely to allow significant amounts of therapeutics to enter the brain. This is further complicated by the highly infiltrative nature of high-grade gliomas which results in the presence of tumorous cells far outside of the disrupted BBB (Van Tellingen O et al., Drug Resistance Updates. 2015 Mar. 1; 19:1-2).

One method that can be employed to overcome the BBB and BBTB is the use of convection-enhanced delivery (CED) proposed by Bobo et al. (Proceedings of the National Academy of Sciences. 1994 Mar. 15; 91 (6): 2076-80). CED utilizes pressure-driven flow to deliver large amounts of therapeutics directly to the brain. In CED, a small caliber catheter is placed through a burr hole in the skull and deployed directly into the brain parenchyma (Bobo RH et al., Proceedings of the National Academy of Sciences. 1994 Mar. 15; 91 (6): 2076-80). CED appeared to be an extremely promising treatment modality through pre-clinical (Kaiser M G et al., Neurosurgery. 2000 Dec. 1; 47 (6): 1391-9; Degen J W et al., Journal of neurosurgery. 2003 Nov. 1; 99 (5): 893-8) and Phase I and II clinical trials (Laske D W et al., Nature medicine. 1997 Dec; 3 (12): 1362-8; Rand R W et al., Clinical Cancer Research. 2000 Jun. 1; 6 (6): 2157-65; Weber F W et al., InLocal Therapies for Glioma Present Status and Future Developments 2003 (pp. 93-103); Lidar Z et al., Journal of neurosurgery. 2004 Mar. 1; 100 (3): 472-9); however, the Phase III (PRECISE) clinical trials failed to meet their clinical endpoints (Kunwar S et al., Neuro-oncology. 2010 Aug. 1; 12 (8): 871-81). A retrospective study conducted by Sampson et al. concluded that drug delivery to tissue at risk for tumor recurrence was inadequate with drug coverage of only 20.1% of the 2 cm tumor margins (Journal of neurosurgery. 2010 Aug. 1; 113 (2): 301-9). The poor drug coverage, aggressive endpoints, and a better than expected response from the control arm combined to result in the failure of the only completed Phase III CED clinical trial for treatment of GBM to date.

As a result of Sampson et al.'s findings, focus was placed on the improvement of catheter design. From this, attention was placed on two major design areas: 1) reflux preventing catheters, and 2) multi-port catheters. While less popular for the treatment of GBM, some groups have proposed catheter modifications in order to create elongated infusion geometries specifically to target the putamen for treatment of Parkinson's Disease. Breeze et al. was the first to propose the use of catheter retraction while infusing fetal tissue for the treatment of Parkinson's Disease (Neurosurgery. 1995 May 1; 36 (5): 1044-8). A similar technique employing the deposition of multiple payloads of therapeutics along the same infusion tract has been used in some clinical trials (Marks Jr W J et al., The Lancet Neurology. 2008 May 1; 7 (5): 400-8; Marks Jr W J et al., The Lancet Neurology. 2010 Dec. 1; 9 (12): 1164-72; Bartus R T et al., Movement Disorders. 2011 January; 26 (1): 27-36). The efficacy of the multiple payload approach was tested by Sillay et al. where the authors concluded that advancement of the catheter resulted in less backflow and a larger infusion radius than retraction of the catheter (Stereotactic and functional neurosurgery. 2013; 91 (3): 153-61). A similar technique, coined “infuse-as-you-go” was recently proposed, which utilizes a multi-step advancement of the catheter in order to cover nearly 15% more of the elongated putamen geometry in non-human primates (Sudhakar V et al., Journal of neurosurgery. 2019 Jul. 12; 133 (2): 530-7). In a similar attempt, Lewis et al. proposed the use of varying catheter step length inducing controlled reflux to create an elongated infusion geometry and increase the volume dispersed (Va) based on the step length (Journal of neuroscience methods. 2018 Oct. 1; 308:337-45). It was recently shown that controlled catheter retraction at a rate of 0.25 mm/min can result in an increase in V_{d} by 50% in an agarose gel model (Mehta JN et al., Pharmaceutics. 2020 August; 12 (8): 753). Additionally, it was shown that the use of constant pressure infusions coupled with controlled catheter retraction can result in an increase in V_{d} of 94% and 143% with 0.25 mm/min and 0.5 mm/min catheters respectively.

The following study uses a computational model to parametrically understand the role of catheter retraction rate on the overall efficacy of controlled catheter movement in CED infusions. To this end, a multi-physics model is first developed and validated, allowing for the estimation of V_{d} of indigo carmine dye in an agarose gel brain tissue phantom. This model is then used to explore the effect of catheter movement rate and distance on V_{d} as well as infusate concentration profile with both constant pressure and constant flow rate infusion conditions.

The materials and methods are now described.

### Biphasic-Solute Material Model

The biphasic-solute material model allows for the transport of a solute in porous media via both diffusion and convection and has consistently been used in the literature to model CED. The material model consists of three constituents, the porous solid matrix (α=s), the solvent (a=w), and the solute (α=u). The model is based on incompressible mixture theory and assumes: 1) each constituent is intrinsically incompressible, 2) negligible inertial effects, 3) isothermal conditions, 4) the solute volume fraction is negligible compared to that of the solid and solvent, and 5) the solute and solvent viscosities are negligible compared to the frictional interactions between constituents. The governing equations describing the balance of linear momentum for the mixture, solvent and solute of a biphasic-solute material was presented by Mauck et al. (J. Biomech. Eng. 2003 Oct. 1; 125 (5): 602-14):

$$div σ = - gradp + div {σ}^{e} = 0 \qquad ( Eq . 6 )$$

$$- {φ}^{w} gradp + {φ}^{w} R θ gradc + {f}_{w s} ( {v}^{s} + {v}^{w} ) + {f}_{w f} ( {v}^{u} - {v}^{w} ) = 0 \qquad ( Eq . 7 )$$

$$- {φ}^{w} R θ {gradc}^{f} + {f}_{s u} ( {v}^{s} - {v}^{w} ) + {f}_{w u} ( {v}^{w} - {v}^{u} ) = 0 \qquad ( Eq . 8 )$$

where σ is the Cauchy stress tensor, p is the interstitial fluid pressure, σ^{e} is the stress caused by the strain in the solid matrix, φ^{α} is the volume fraction of α, R is the universal gas constant, θ is the absolute temperature, c is the concentration of the solute, and f_{αβ} is thee diffusive drag force between the α and β. Next, Ateshian et al. (Journal of biomechanics. 2006 Jan. 1; 39 (3): 464-75) synthesized the non-zero diffusive drag coefficients as:

$${f}_{w s} = \frac{{( {φ}^{w} )}^{2}}{k} , {f}_{wu} = \frac{{φ}^{w} R θ c}{{D}_{0}} , {f}_{wu} + {f}_{su} = \frac{{φ}^{w} R θ c}{D} \qquad ( Eq . 9 )$$

where k is the solvent hydraulic permeability in the solid matrix, D is the diffusivity of the solute in the mixture, and D₀ is the diffusivity in free solution. The mass balance of the mixture, solute, and solid are:

$$d i v ( {v}^{s} - w ) = 0 \qquad ( Eq . 10 )$$

$$\frac{\partial ( {φ}^{w} c )}{\partial t} + d i v ( j + {φ}^{w} {cv}^{s} ) = 0 \qquad ( Eq . 11 )$$

$${φ}^{s} = \frac{{φ}_{r}^{s}}{J} \qquad ( Eq . 12 )$$

where v^{α} is the velocity of constituent α, W=(v^{w}−v^{s}) is the volumetric flux, j=φ^{w}c(v^{u}−v^{s}) is the molar flux, of is the solid volume fraction in the reference state and J=det F where F=I+grad u, where u is the displacement vector. Eq. 6, Eq. 10, and Eq.11 are the three governing partial differential equations that can be used to solve for the displacement vector, u, the interstitial fluid pressure, p, and the solute concentration, c. Additionally, Eq.7 and Eq.8 can be inverted in order to obtain relations for the solvent and solute fluxes respectively as a function of their driving forces as presented by Ateshian et al. (Journal of biomechanical engineering. 2011 Aug. 1; 133 (8)). Lastly, the use of the effective pressure, where \tilde{p}=p−Rθφc, and effective concentration, where

$$\tilde{c} = \frac{c}{\tilde{κ}} ,$$

in place of the actual pressure, p, and concertation, c, allows for continuity to be enforced across boundaries yielding a valid finite element framework. However, it should be noted that the model assumes ideal solubility (\tilde{k}=1) so the effective concentration reduces to the actual concentration.

### Material Model and Boundary Conditions

In order to conduct a parametric study on needle retraction speed, a material model similar to what was presented by Elenes et al. (Journal of Engineering and Science in Medical Diagnostics and Therapy. 2019 Aug. 1; 2 (3)) was used. Briefly, the solid matrix was modeled as agarose gel with a solid volume fraction of 0.6% (w/w). A neo-Hookean material with a Poisson's ratio of 0.35 and an elastic modulus of 6000 Pa was used to represent the agarose gel. The neo-Hookean material is an extension of Hooke's law formulated for large deformations. The Holmes-Mow relation for the strain-dependent hydraulic permeability tensor was reduced to form the relation proposed by Lai and Mow (Biorheology. 1980 Jan. 1; 17 (1-2): 111-23), by setting β=0 in Eq. 13:

$$k = {{k}_{0} ( \frac{J - {φ}_{0}}{1 - {φ}_{0}} )}^{α} {e}^{\frac{1}{2} M ( {J}^{2} - 1 )} \qquad ( Eq . 13 )$$

where k₀ is the isotropic hydraulic permeability and given the lack of experimental data, the parameter was determined parametrically and set at 3.5 mm⁴N⁻¹s⁻¹ which is of the same order of magnitude used previously (Elenes EY et al., Journal of Engineering and Science in Medical Diagnostics and Therapy. 2019 Aug. 1; 2 (3)). The non-linear parameter M was set to 1, consistent with the range presented in other models (Elenes EY et al., Journal of Engineering and Science in Medical Diagnostics and Therapy. 2019 Aug. 1; 2 (3); Chen X et al., Annals of Biomedical Engineering. 2007 Dec. 1; 35 (12): 2145-58; García JJ et al., Annals of biomedical engineering. 2009 Feb. 1; 37 (2): 375; Sobey I et al., Mathematical Medicine and Biology. 2006 Dec. 1; 23 (4): 339-61). Further, the solvent was modeled as indigo carmine dye with a molecular weight of 466 Da, a free diffusivity (D₀) of 3.75×10⁻⁴ mm²s⁻¹, an effective diffusivity (D) of 2.8×10⁻⁴ mm²s⁻¹, and ideal solubility. Effective diffusivity was determined parametrically, and free diffusivity was selected to be in the range of diffusivity constants predicted by the Stokes-Einstein relation, Wilke-Chang relation (Chang P et al., The Journal of Physical Chemistry. 1955 July; 59 (7): 592-6), and the relation proposed by Reddy and Doraiswamy (Reddy K A et al., Industrial & Engineering Chemistry Fundamentals. 1967 February; 6 (1): 77-9) using the known properties of indigo carmine dye, water, and a temperature of 25° C.

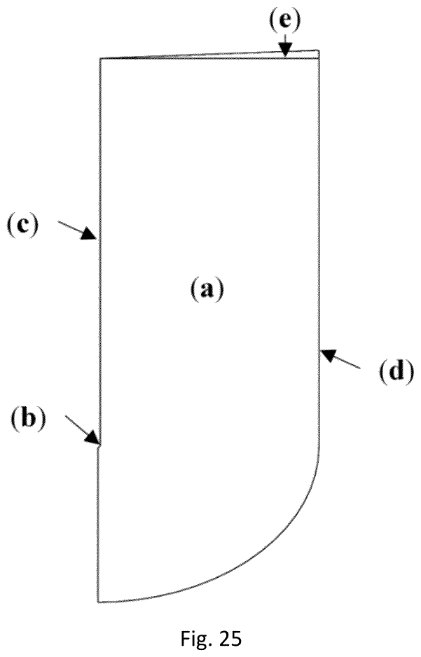

Similar to the geometry presented by Orozco et al. (Orozco GA et al., Revista Ingeniería Biomédica. 2014 December; 8 (16): 56-64; Orozco GA et al., Medical & biological engineering & computing. 2014 October; 52 (10): 841-9), a solid model of the porous matrix was created consisting of a semicircular initial infusion cavity (r=0.180 mm), corresponding to the outer diameter of the catheter used in the experiment, and a 12 mm semicircular outer boundary beneath a 48 mm tall cylindrical surface, as shown in FIG. 25(a). The two-dimensional surface was revolved by 3° in order to create a three-dimensional geometry that could be modeled as a pseudo two-dimensional feature in FEBio. The model was then imported into Gmsh (version 4.4.1, gmsh.info) where a three-dimensional mesh was created using a combination of 8-node trilinear hexahedral and 6-node linear pentahedral elements. The mesh was biased toward the infusion cavity and contained more than 45k elements as this mesh density resulted in an error of less than 2% of the maximum prescribed concentration for all simulations. Additionally, models with this mesh density resulted in less than 5% difference in calculated flow rate compared to a mesh with a 110% increase in density. Next, the model was imported into Preview (version 2.1.5, www.febio.org) in order to create the geometry input file for simulation.

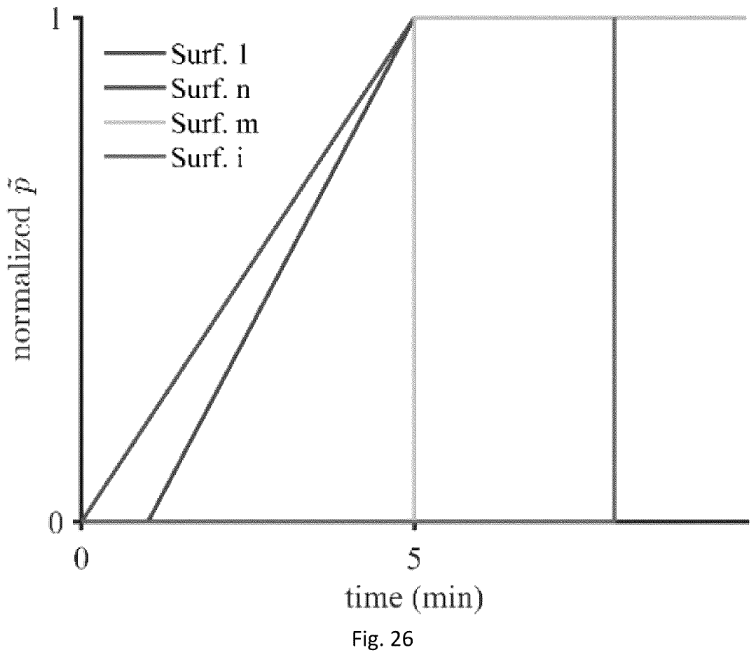

A script was written in Matlab (R2018a, Natick, MA) in order to create the boundary conditions on each surface. Pressure and concentration were applied to the initial infusion cavity (FIG. 25(b)), as well as portions of the catheter cavity (FIG. 25(c)) to model needle movement. In order to best model the conducted experiment, the initial infusion surface included the spherical infusion cavity along with 2 mm of the vertical infusion surface. This was done in order to model the void left by the 2 mm retraction step conducted directly after catheter insertion used to remove any agarose that may have become stuck on the catheter tip. Concentration was applied instantaneously, while effective pressure was applied linearly over the first 5 minutes of the simulation for surfaces that were active in the first 5 minutes and effective pressure was applied instantaneously for surfaces that became active after 5 minutes, as shown in FIG. 26. The linear application of pressure was chosen because measured rise time for pressure was 4.5 minutes in the experiments. Additionally, a zero radial contact stress boundary condition (σ^{e}=0) was applied to all free surfaces on the solid matrix at the intersection between the fluid and the porous matrix. Zero-pressure boundary conditions were applied to the outer matrix boundary (FIG. 25(d)) and zero displacement, fluid flux, and mass flux were applied to the top surface (FIG. 25(e)) as well as to the symmetry faces. In order to create needle movement, a multistep analysis was used in which each needle movement was considered a new analysis step. The initiation time of each new step was determined by the retraction rate and the mesh size. This allowed a new set of boundary conditions to be applied at the initiation of each analysis step allowing for infusion to begin to move up along the needle tract, as shown in FIG. 27. The vertical mesh size along the movement surfaces was 0.05 mm; however, catheter movement discretization was 0.5 mm in order to reduce computational cost. The total retraction distance permitted was 25 mm in order to correspond with a maximum initial deployment distance of 30 mm. The arising boundary value problem was solved using FEBio (version 2.9.1, www.febio.org).

### Constant Pressure Adjusted Model

An adjusted model was also created for analysis. This model used the same boundary conditions already stated and added boundary conditions to decrease pressure through the length of the infusion cavity as a function of the distance from the needle tip. This allowed for the reduction in pressure expected as the infusate traveled through the cavity. Additionally, the initial infusion cavity size was reduced from 0.18 mm to 0.09 mm. An initial analysis step of 1 second was added where the infusion cavity was expanded to 0.18 mm this was done in order to represent the compressive stress on the tissue caused by the needle. The choice of creating an initial cavity radius of 0.09 mm also allows for the representation of tissue damage caused by needle insertion. Infusion was started as normal at the 1 second mark, and the cavity shrinking, which is expected to occur when the catheter is no longer in a position to force the cavity open, was enforced as a function of needle tip distance from each surface along the cavity. The decrease in pressure and cavity size began at locations greater than 2 mm away from the current needle tip location and reached a minimum at distances of 10 mm away from the current needle tip locations.

### Constant Flow Rate Model

Constant infusion flow rates, as typically prescribed in CED infusions, cannot be applied without prior knowledge of infusion fluid flux and either the deformation caused by the infusion pressure or the fluid velocity. In order to overcome this challenge, many models use constant pressure infusion protocols (Elenes EY et al., Journal of Engineering and Science in Medical Diagnostics and Therapy. 2019 Aug. 1; 2 (3); Chen X et al., Annals of Biomedical Engineering. 2007 Dec. 1; 35 (12): 2145-58; García JJ et al., Annals of biomedical engineering. 2009 Feb. 1; 37 (2): 375) while other models have created a constant flow model by restricting the deformation of the tissue along the infusion cavity (García JJ et al., Journal of Computational and Nonlinear Dynamics. 2013 Jan. 1; 8 (1)). The flow rate of infusate into the tissue is based on two components: 1) flow into tissue due to fluid flux, and 2) flow into tissue due to deformation of the infusion cavity. The first component can be determined using Eq. 14:

$${V}_{flu} = {\sum }_{i = 1}^{n} {A}_{i} {w}_{i} Δ t \qquad ( Eq . 14 )$$

where V_{flu} is the volume of infusate into the tissue due to flux, Aᵢ is the surface area of each element along the infusing surface, wᵢ is the fluid flux at each element along the infusing surface, and At is the step size at the time of interest. The second component can be calculated as the change in volume of the infusion cavity from the previous time step to the current time step, this can be approximated by computing the difference of the convex hull of the infusion surface for the two adjacent time steps. Next, the total volume of fluid into the tissue can be computed by summing the two volumes as shown in Eq. 15:

$${V}_{tot} = {V}_{C H} + {V}_{flu} \qquad ( Eq . 15 )$$

where Vₜₒₜ is the total infused volume over the time step and V_{CH} is the change in infusion cavity volume. Finally, the volumetric flow rate, Q, can be computed as shown in Eq. 16:

$$Q = \frac{{V}_{t o t}}{Δ t} \qquad ( Eq . 16 )$$

the constant infusion pressure method, was shown to be a fair assumption when using a stationary catheter (Elenes EY et al., Journal of Engineering and Science in Medical Diagnostics and Therapy. 2019 Aug. 1; 2 (3)), as tissue deformation is relatively small making V_{CH} negligible after the initial time steps. Further, the surface area, Aᵢ, over which the infusion occurs stays constant after initial deformation allowing for a reasonable calculation of average flow rate as a proxy for the infusion flow rate. However, this method of calculating average flow rate does not hold when a catheter begins to move because the surface area over which flow is occurring grows significantly.

To overcome the inability of a constant infusion pressure to model a constant flow rate, a searching algorithm was used to find the required applied effective pressure that would result in the target flow rate for each time step. An initial guess for the effective pressure is applied instantaneously at the beginning of each analysis time step and the model is executed for that single step. Next, the flow rate is calculated using Eq. 16 and compared to the target flow rate. The error between the target and computed flow rate is used to update the guess for applied effective pressure and the analysis step is re-run with the new pressure. When the difference in applied effective pressure is within 1 Pa between successive iterations, the problem is considered converged and the algorithm ends. The final effective pressure then becomes the optimal pressure for that time step and the algorithm restarts at the next time point. As a result, the prescribed infusion pressure becomes time dependent and yields a nearly constant infusion flow rate. All other boundary conditions are chosen to be the same as the baseline constant pressure model.

### Model Validation

In order to validate these models, results obtained from constant pressure experiments conducted in agarose gel were compared. This experiment utilized catheters moving at rates of 0, 0.25, and 0.5 mm/min to infuse indigo carmine dye at an infusion line pressure of 977 Pa (constant pressure infusions) corresponding to the average pressure required to create a flow rate of 1 μL/min with a stationary catheter or a flow rate of 1 μL/min (constant flow rate infusions) for 100 min. The V_{d} previously reported was compared to the V_{d} estimated by the computational model at the same concentration threshold of 0.05%.

V_{d} was calculated by taking all nodes above the concentration threshold of 0.05% of the prescribed concentration. Artifacts from the mesh discretization were removed by linearly interpolating between the last row of nodes above the concentration threshold and the first row of nodes below the threshold in order to find the approximate location the concentration threshold.

Next, the convex hull of the linearly interpolated nodes was taken to find the volume of the pseudo 2-dimensional infusion. This infusion volume could be multiplied by 120 in order to get the entire 3-dimensional V_{d} and compared to the experimental results. The model was validated by comparing V_{d} between the experimental results and the computational model results at 20-minute intervals. The V_{d} of the experiment was plotted against the V_{d} of the model and a best fit line of y=x was imposed on the data. The model was then considered validated if the R-squared value of the best fit line was greater than 0.9, suggesting good agreement between the model and the experiment. After validation, a parametric analysis was conducted exploring different retraction rates ranging from 0 to 1 mm/min.

The results are now described.

### Model Validation

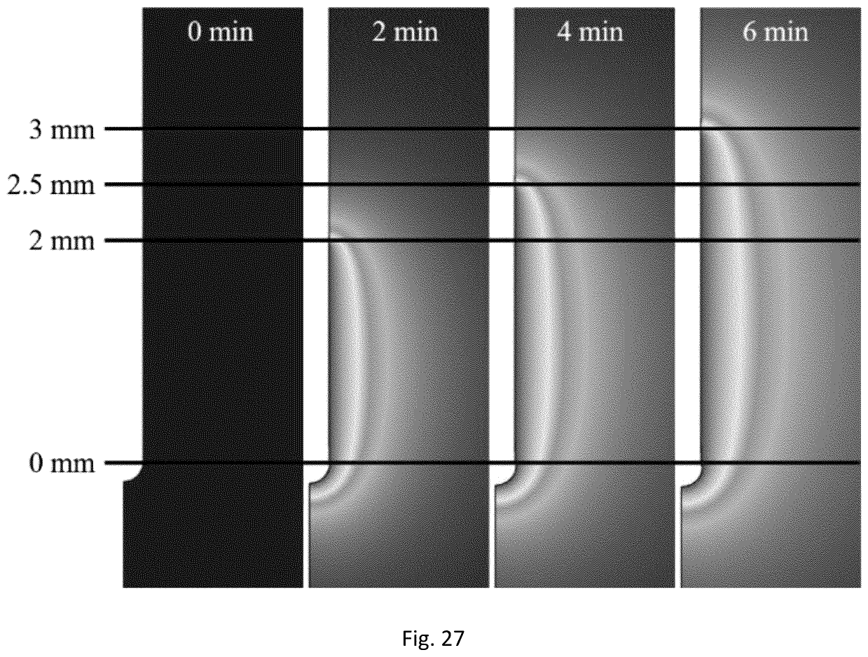

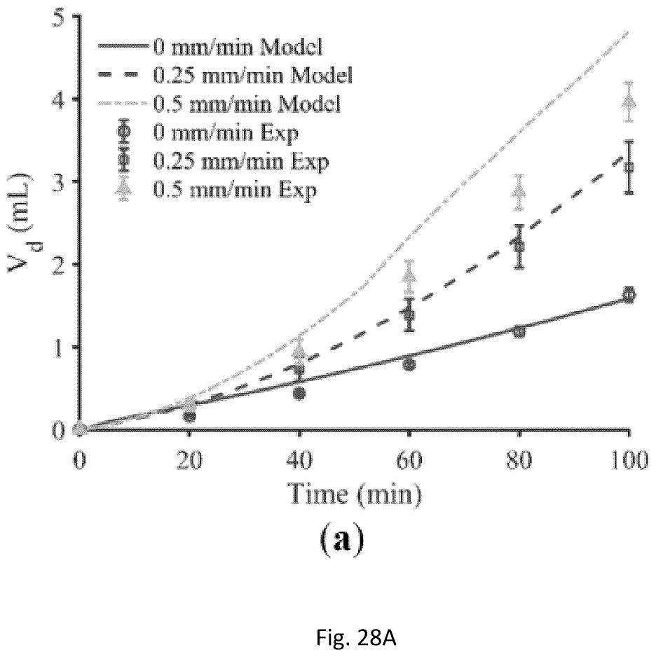

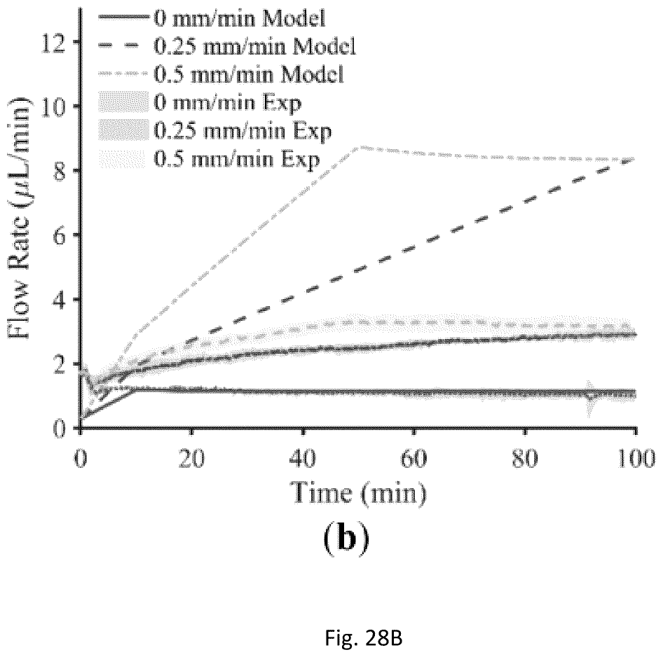

Results from the baseline computational model with no decrease in pressure based on catheter tip position and no shrinking of the infusion cavity is shown in FIG. 28. An infusion pressure of 850 was selected parametrically to obtain good agreement with the flow rate of the stationary catheter found in the experiment, given a lack of experimental data for pressure inside of the infusion cavity. Predicted V_{d} for the stationary and 0.25 mm/min retracting catheter is near the experimental values, but predicted V_{d} of the 0.5 mm/min catheter is larger than that of the experiment. Further, the flow rate of the 0 mm/min catheter is nearly identical to the flow rate prescribed by the syringe pump in the experiment averaging 1.11 μL/min for both the experiment and the model, but becomes quite different for the 0.25 and 0.5 mm/min catheters with average flow rates of 5.11 μL/min and 6.94 μL/min respectively. These values do not compare similarly to the constant pressure experiments which resulted in average flow rates of 2.41 μL/min and 2.90 μL/min for the 0.25 and 0.5 mm/min retraction rates respectively.

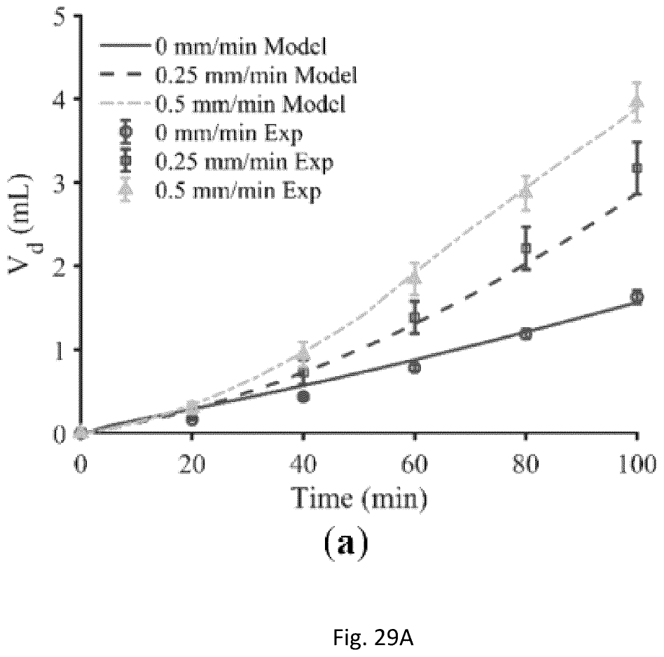

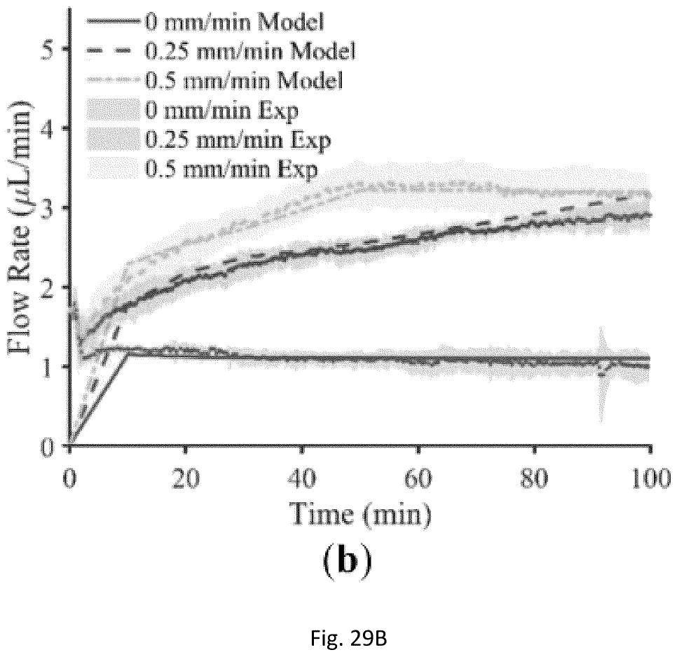

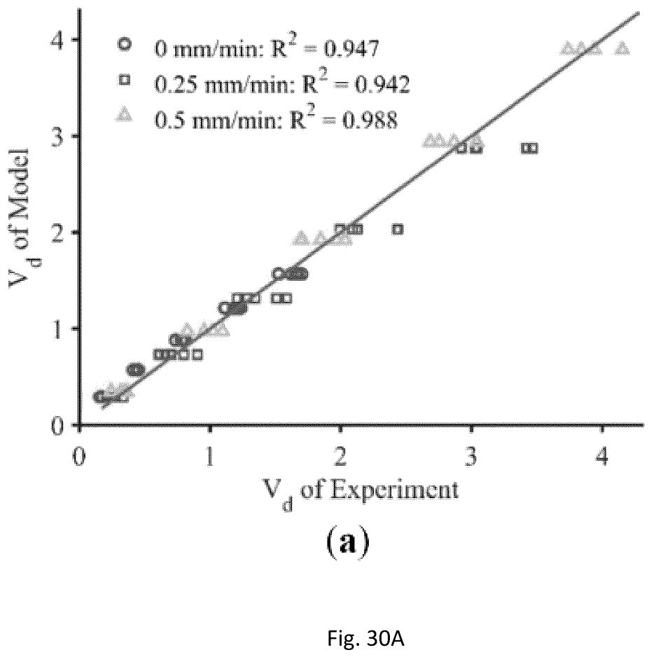

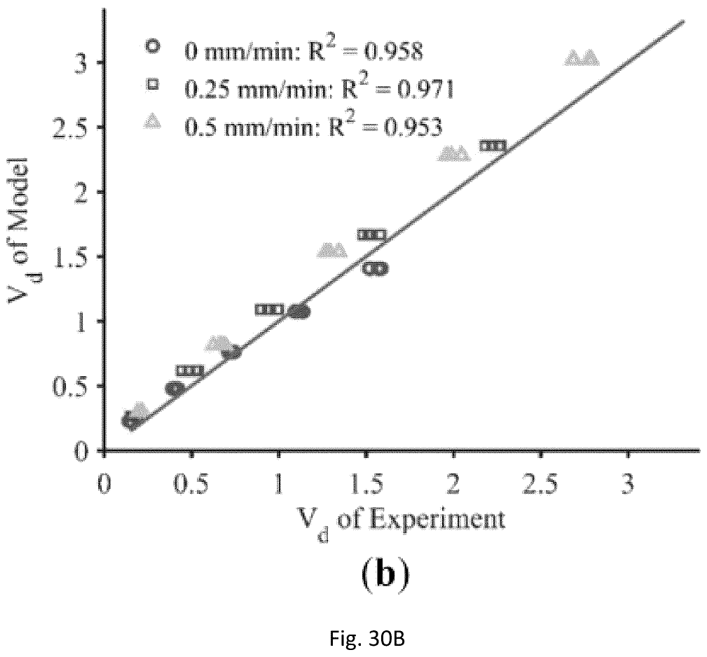

The adjusted constant pressure computational model used an initial infusion cavity pressure of 850 Pa which was decreased linearly according to the distance from each surface to the needle tip. Pressure started decreasing on surfaces greater than 2 mm away from the needle tip and reached a minimum pressure of 200 Pa at distances larger than 10 mm from the needle tip. Similarly, the infusion cavity radius reduced from 0.18 mm to 0.09 mm over the same distance. FIG. 29 shows the time varying V_{d} and flow rate predicted by the computational model along with the actual V_{d} calculated from the agarose gel experiments. The mean flow rates of the 0 mm/min, 0.25 and 0.5 mm/min catheters more closely resemble that of the experiment with average flow values of 1.11, 2.55, and 2.93 μL/min with the adjusted model. FIG. 30 shows the V_{d} of the experiments plotted against the model for each retraction rate with a fit line of y=x. Coefficients of determination of greater than 0.9 for all threes movement rates suggest good quantitative agreement between the model and the experiment.

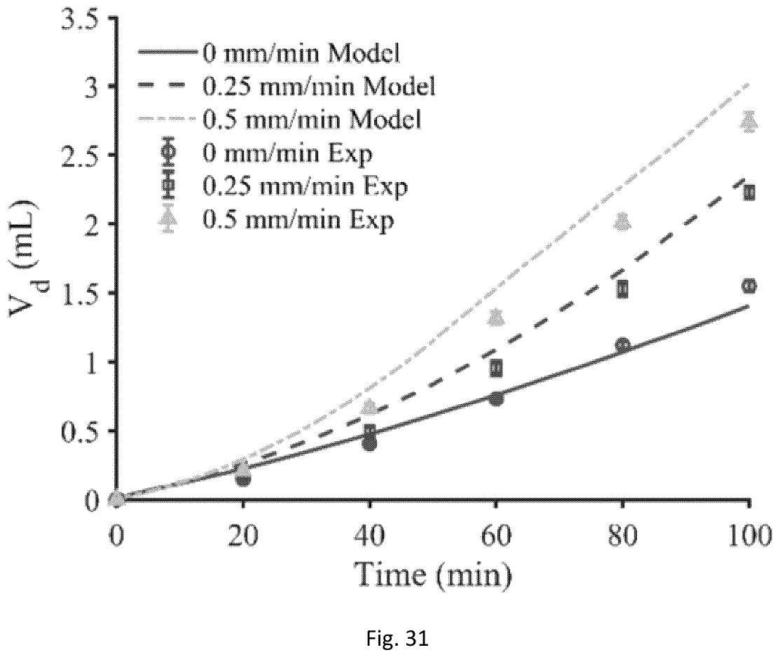

Finally, FIG. 31 shows the time varying V_{d} predicted by the constant flow rate computational model along with the actual V_{d} calculated from the agarose gel experiments. The flow rate of the computation model is within 1% of the prescribed flow rate of the experiment for all time points. FIG. 30(b) shows the V_{d} of the experiments plotted against the model for each retraction rate with a fit line of y=x. Coefficients of determination of greater than 0.95 for all movement rates suggest good quantitative agreement between the model and the experiment.

### Volume Dispersed-Retraction Rate

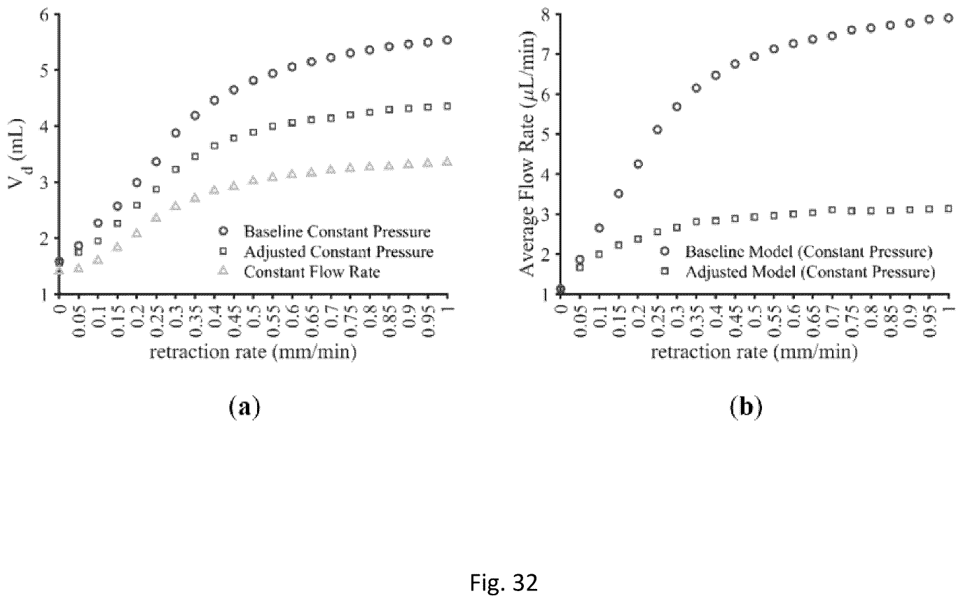

The baseline constant pressure, adjusted constant pressure, and constant flow rate models can be extended to explore retraction rates other than those used in the experiments. FIG. 32(a) shows the V_{d} for catheter retraction rates of between 0 and 1 mm/min for the baseline constant pressure, adjusted constant pressure, and constant flow rate models. FIG. 32(b) shows the average flow rate achieved for the baseline and adjusted constant pressure models, the constant flow rate model achieved a flow rate within 1.5% of the prescribed 1 μL/min for all retraction rates at all time points. Since maximum retraction distance was limited to 25 mm, the flow rate and V_{d} do not continue to increase in large amounts at higher retraction rates. V_{d} appears to increase the most between the 0 and 0.3 mm/min catheters for the constant pressure models and between 0.05 mm/min and 0.25 mm/min catheters for the constant flow rate model with at least 10% change in V_{d} between successive retraction rates.

### Volume Dispersed-Movement Distance

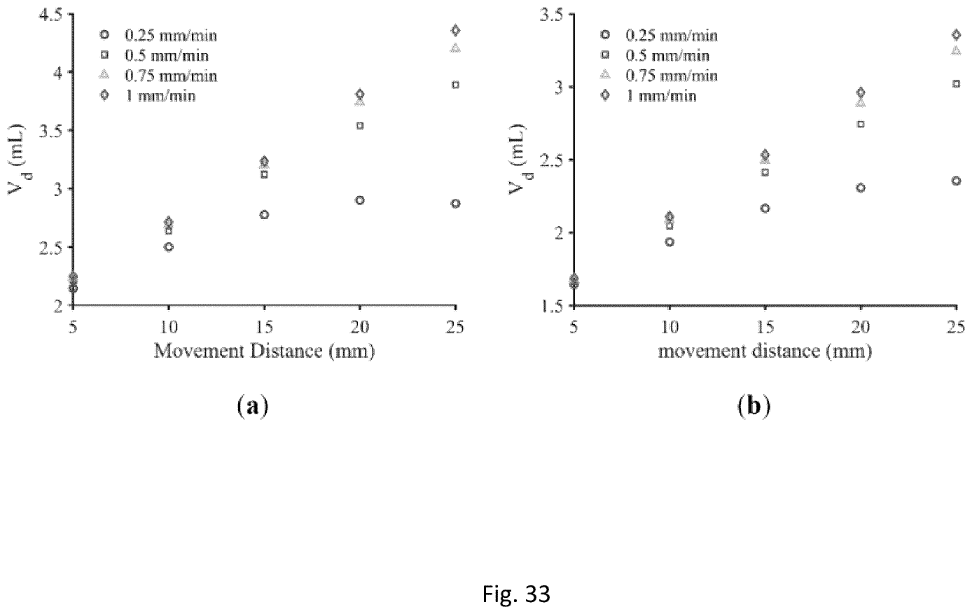

The total movement distance was varied between 5 mm and 25 mm in 5 mm increments for the 0.25, 0.5, 0.75, and 1 mm/min catheters using both the adjusted constant pressure and the constant flow rate models. FIG. 33(a) and(b) show the total V_{d} calculated after 100-minute infusions with the various movement distances and retraction rates. At shorter movement distances, retraction rate has a small impact on total V_{d}, with the impact increasing with increasing movement distance. Further, the movement distance that allows continuous movement throughout the infusion appears to have little advantage over a movement distance that would cause a brief stop in retraction at the end of the infusion. This is especially the case in the constant pressure infusion with a movement distance of 25 mm and retraction rate of 0.25 mm/min which results in a 1% decrease in V_{d} compared to the 20 mm movement distance using the same retraction rate.

### Infusion Profiles

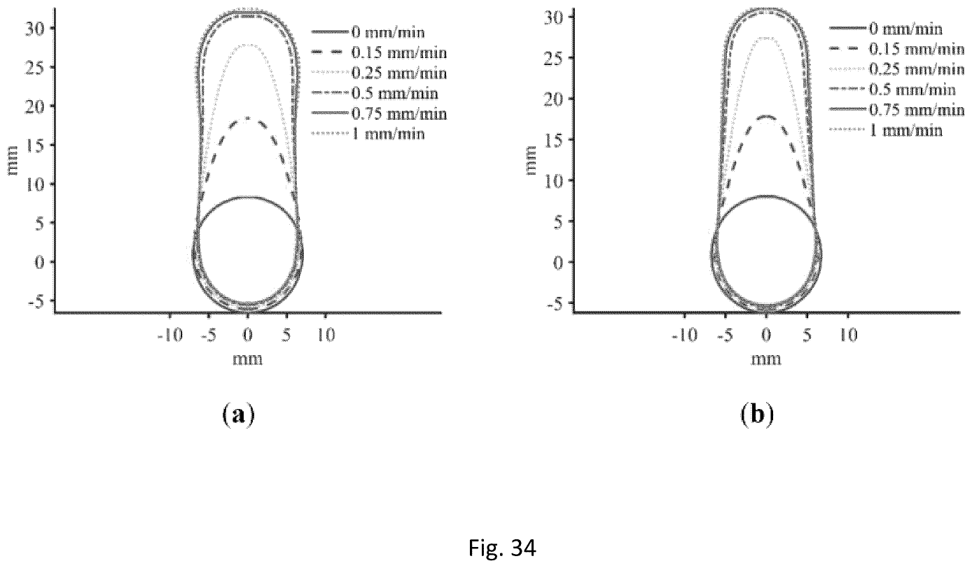

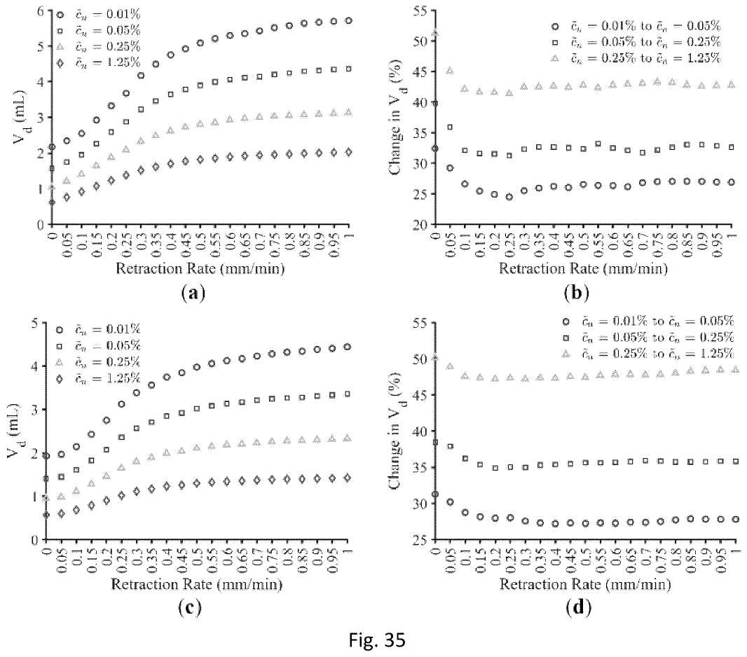

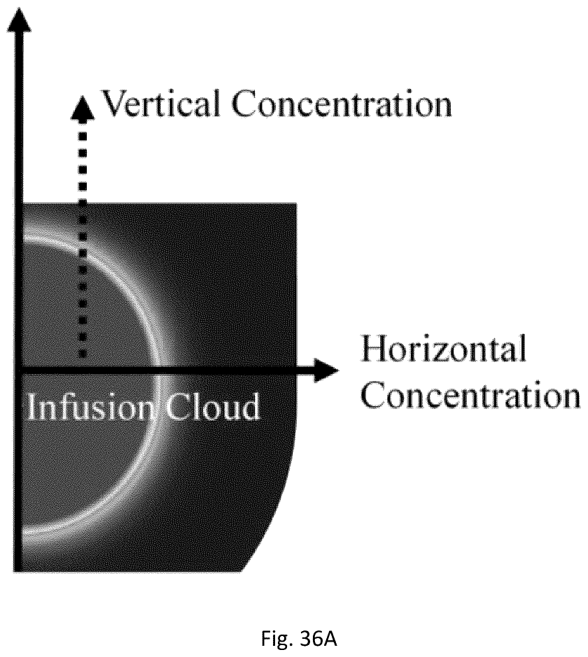

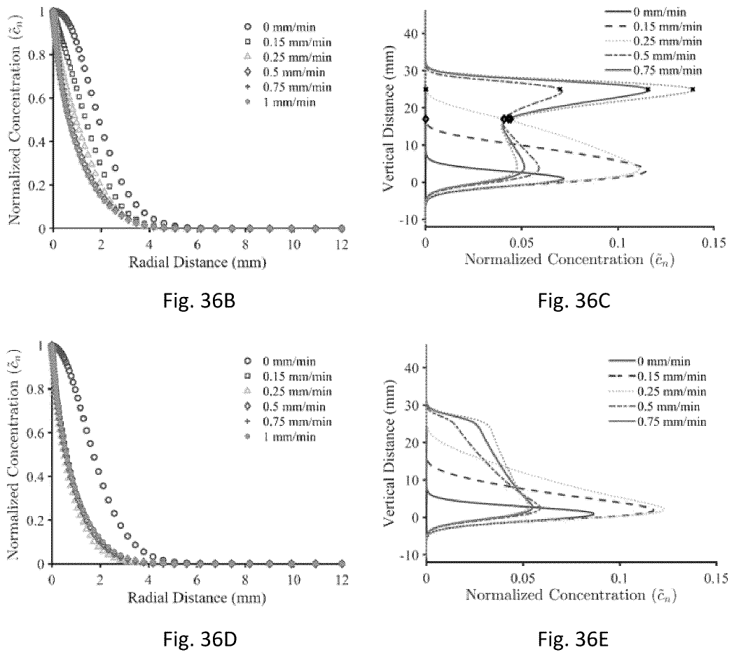

Plots showing the infusion morphology for the adjusted constant pressure and constant flow rate models at a concentration threshold of 0.05% are shown in FIG. 34. As seen in FIG. 34, retraction rate appears to have an impact on infusion shape. Additionally, the normalized effective concentration appears to be skewed toward lower concentrations for all rates. Surprisingly, the 0 mm/min catheter resulted in the most skewed concentration curve with concentration profiles leveling off at and above 0.25 mm/min rates. FIG. 35(a) and(c) show the impact that the variation in concentration profile has on calculated V_{d} for the adjusted constant pressure and constant flow rate models respectively. FIG. 35(b) and(d) show the percent change in V_{d} that occurs for each retraction rate when using successive threshold concentrations for the adjusted constant pressure and constant flow rate models respectively. In order to examine the concentration distribution both radially and vertically, concentration was measured through a standard line, as shown graphically in FIG. 36A. The horizontal line was chosen to go through the midpoint of the infusion cloud and the vertical line was chosen to go through the radial midpoint of the cloud in order to capture the reduction in concentration at locations away from the infusion cavity. The concentration distribution along the initial radius of the infusion cloud at 100 minutes is shown in FIG. 36B and FIG. 36D for the adjusted constant pressure and constant flow rate models respectively and the concentration along the vertical axis at half of the 0.05% concentration threshold radius for the respective models is shown in FIG. 36C and FIG. 36E.

The baseline computational model resulted in the largest V_{d} and flow rates with catheter retraction, with flow rate increasing linearly until the catheter stopped moving at a distance of 25 mm. While advantageous, this model does not appear to capture the true conditions inside of the cavity during catheter retraction. Once the catheter begins to move, it is likely that the infusion cavity will reduce in size since the needle is no longer in position to force the cavity open. Additionally, it is unlikely that pressure will be uniform through the entire cavity as the pressure source is located at the proximal end of the cavity, where the catheter tip is located. Given this, it follows that pressure would decrease along the cavity length similar to flow through a cylinder, but because of the short cavity length, this pressure loss may be negligible. What is more likely a significant loss in pressure is the loss of infusate through the porous media as the infusate travels down the cavity, resulting is a similar issue, albeit less exaggerated, issue found by Raghavan et al. where fluid flows primarily through the proximal port in multiport catheters (Neurosurgical focus. 2006 Apr. 1; 20 (4):E12). Nonetheless, this model suggests the possibility of great increases in V_{d} with pressure driven flow if uniform pressure and cavity size can be ensured. A possible method of retaining cavity size while increasing the surface are over which infusion can occur is the use of multiport catheters which contain multiple openings for infusion along length of the catheter. While these catheters preferentially infuse through the most proximal opening, modifications can be made to reduce this occurrence (Raghavan R et al., Neurosurgical focus. 2006 Apr. 1; 20 (4):E12). One example is the hollow-fiber catheter which utilizes much smaller ports thereby increasing the resistance to flow and allowing a more uniform infusion from all of the ports (Seunguk OH et al, Journal of neurosurgery. 2007 Sep. 1; 107 (3): 568-77). While this method showed a nearly 3-fold increase in V_{d}, the experiments were limited to a very low flow rate of 0.1 μL/min (Seunguk OH et al, Journal of neurosurgery. 2007 Sep. 1; 107 (3): 568-77). The addition of multiple small ports along the catheter appears to not only increase surface area for CED but also likely decreases infusion pressure due to this increase in surface area, a finding which could further benefit from constant pressure infusions. Similarly, the use of controlled reflux, proposed by Lewis et al. (Journal of neuroscience methods. 2018 Oct. 1; 308:337-45) works under a similar principle to the multiport and controlled catheter movement protocols where an increase in infusion surface area results in an increase in Va; and therefore, may not only benefit from constant pressure infusions but may be advantageous due to its ability to keep the cavity open throughout the entire infusion.

Both the baseline and the adjusted constant pressure models show the largest increase in V_{d} occurring between the 0 mm/min and 0.3 mm/min retraction rates while the constant flow rate model was more sensitive to movement rate with the highest increase occurring between 0.05 and 0.25 mm/min retraction rates. Additionally, retraction rates that cause the catheter to stop moving before the end of an infusion result in an increasingly marginal gain in Va. What is more, it is clear from the experiments that some interaction between the catheter and the infusion cavity is taking place, so increasing retraction rates may result in even less gain in V_{d} than predicted here.

Variation in total movement distance reveals a less sensitive V_{d} response to retraction rate with lower movements and an increase in sensitivity with larger movements. Interestingly, it appears that using a retraction rate that would result in a brief period of catheter pause at the end of the infusion may have a small V_{d} advantage compared to moving the catheter throughout the infusion. Stopping the infusion at the same time that retraction is stopped may not allow for sufficient time for the most proximal surfaces to result in appreciable radial infusion, resulting in less than desired infusion volumes. This phenomenon also explains why the 0.3 and 0.35 mm/min retraction rates appear to be part of the mostly linear region in the plots shown in FIG. 32.

While all retraction rates result in rapid infusate concentration decay away from the infusion cavity, the stationary catheter appears to have the quickest decay. On closer inspection, this result is due to the quicker attenuation in concentration along the vertical axis. Given this, the stationary catheter is less robust to variations in minimum concentration requirements. A similar bias toward lower concentrations appears to be seen in infusions of the contrast agent iohexol in ex vivo porcine brains, where nearly 40% of voxels had a concentration between 10 and 15% (Chen X et al., Annals of Biomedical Engineering. 2007 Dec. 1; 35 (12): 2145-58). This is further supported by the “square” curves typically used to describe the concentration gradient of CED infusions (Elenes EY et al., Journal of Engineering and Science in Medical Diagnostics and Therapy. 2019 Aug. 1; 2 (3); García JJ et al., Annals of biomedical engineering. 2009 Feb. 1; 37 (2): 375). On the other hand, any catheter movement is more robust in this regard and does not appear to experience as significant of a change in V_{d} when concentration threshold is changed, due to a larger variation in concentration.

The choice of retraction rate also appears to have an impact on the final shape or morphology of the infusion. The stationary catheter results in a nearly spherical infusion. It should be noted that the stationary infusion protocol did not result in a perfectly spherical shape due to the choice to use a 2 mm long initial infusion cavity, resulting in a mildly elongated infusion shape. Slow to moderate retraction rates result in a teardrop shaped infusion and quick moving catheters, with retraction rates above about 0.5 mm/min, appear to result in mostly cylindrical infusions. Therefore, the choice of retraction rate may depend on the shape of the target area or the type of catheter used. For example, in the CED treatment of Parkinson's disease, the putamen is generally targeted which has an elongated shape (Bankiewicz KS et al., Journal of Controlled Release. 2016 Oct. 28; 240:434-42), in this case a quick moving catheter may be most advantageous in efficiently covering the entire region of interest. Additionally, the use of a catheter like the arborizing catheter (Elenes EY et al., Journal of Engineering and Science in Medical Diagnostics and Therapy. 2021 Feb. 1; 4 (1): 011003; Elenes EY. An arborizing, multiport catheter for maximizing drug distribution in the brain via convection enhanced delivery (Doctoral dissertation)) may benefit most from a moderate retraction rate, such as 0.25 or 0.3 mm/min. In this case, the long tapered infusion shape may be an advantage due to the geometry of the catheter, which results in larger separation distances between adjacent needles at long deployment lengths and smaller separation distances at shorter deployment distances. Therefore, the use of the moderate retraction rate may allow for effective infusion cloud overlap between adjacent needles without causing excessive overlap which could be inefficient and wasteful.

The flow rates of the retracting catheter in the adjusted model are more similar to that of the experiments, but still exhibit a linear relation between flow rate and time rather than the slightly nonlinear behavior seen in the experiment. Both the degree of actual cavity shrinking and decrease in cavity pressure are not known but were found parametrically.

In this study, a final infusion cavity diameter of 0.09 mm was found to give the best fit to the experimental data. This results in a stretch ratio of 0.5, previous studies in agarose gel showed a stretch ratio of between 0.708 and 0.763 when inserting needles of varying size into 0.6% agarose gel (Casanova F et al., J Biomech Eng. April 2012, 134 (4): 041006) at a rate of 1.8 mm/s. Similarly, a minimum stretch ratio of 0.455 was found with an insertion speed of 0.2 mm/s in a rat brain (Casanova F et al., PLOS One. 2014 Apr. 28; 9 (4): e94919). However, an insertion speed of approximately 13 mm/s was used in the experiments and an increased insertion speed has been shown to increase radial stress in agarose by 226% when comparing an insertion rate of 1.8 mm/s to a rate of 10 mm/s (Urrea FA et al., Journal of the mechanical behavior of biomedical materials. 2016 Mar. 1; 56:98-105), suggesting that the stretch ratio in the experiments should be significantly less than those previously found in agarose.

The constant flow rate model was able to achieve a flow rate within 1.5% of the prescribed flow rate which is likely better than that found in actual experiments due to noise variables such as tubing compliance that is not often controlled for. This framework allows for further extension of the model to explore the effects of variation of constant flow rates on retraction rate and movement distance. This can be accomplished in a similar method presented by Orozco et al (Orozco GA et al., Revista Ingeniería Biomédica. 2014 December; 8 (16): 56-64; Orozco GA et al., Medical & biological engineering & computing. 2014 October; 52 (10): 841-9).

### Example 3: MRI-Compatible Positioning System for Remotely Operating an Arborizing Multiport Catheter

Glioblastomas are the most common type of glioma, accounting for 48 percent of malignant brain tumors and 57 percent of all gliomas (Ostrom QT et al., Neuro-oncology. 2019 October; 21 (Supplement_5): v1-00; Ostrom QT et al., Neuro-oncology. 2015 Oct. 1; 17 (suppl_4): iv1-62). They are also a particularly deadly type of tumor and have a median five-year survival rate of less than seven percent (Ostrom QT et al., Neuro-oncology. 2019 October; 21 (Supplement_5): v1-00; Ostrom QT et al., Neuro-oncology. 2014 Jul. 1; 16 (7): 896-913). The World Health Organization (WHO) categorizes glioblastomas as a Grade IV glioma, meaning they are malignant, diffuse into neighboring tissue, cause necrosis, and are quickly fatal (Louis DN et al., Acta neuropathologica. 2007 August; 114 (2): 97-109). The standard of care for glioblastoma treatment currently consists of a maximal safe surgical resection of the tumor followed by radiotherapy and concomitant and/or adjuvant chemotherapy (Nabors LB et al., J Natl Compr Canc Netw. 2020 Nov. 2; 18 (11): 1537-1570; Wang JL et al., Advances in Biology and Treatment of Glioblastoma. 2017:57-89). While meta-analyses have shown the advantage of multi-modality therapy in the treatment of glioblastoma (Fine HA et al., Cancer. 1993 Apr. 15; 71 (8): 2585-97; Stewart L, The Lancet. 2002 Mar. 23; 359 (9311): 1011-8), median survival remains low at 12 to 15 months (Wen PY et al., Erratum in: N Engl J Med. 2008 Aug. 21; 359 (8): 877).

One of several approaches to improving treatment of glioblastoma is the use of targeted therapies, which involve the use of drugs that take advantage of the growing knowledge of the genomic and molecular characteristics of glioblastoma (Wang JL et al., Advances in Biology and Treatment of Glioblastoma. 2017:57-89). Targeted therapy drugs narrowly target the tumors and are a promising area of research in the treatment of glioblastomas (Wang JL et al., Advances in Biology and Treatment of Glioblastoma. 2017:57-89). However, systemic treatment is challenging as 98% of small molecule therapeutics are unable to penetrate the blood-brain-barrier (BBB) (Pardridge WM, NeuroRx. 2005 January; 2 (1): 3-14) and targeted drugs are even more difficult to deliver across the BBB because they are often polar and have a high molecular weight (Bobo RH et al., Proceedings of the National Academy of Sciences. 1994 Mar. 15; 91 (6): 2076-80). One method of treatment being explored to overcome the obstacle of the blood-brain-barrier is convection enhanced delivery (CED) of targeted drugs. This approach delivers drugs directly to the cancerous region of the brain, using a pressure gradient to improve drug distribution (Bobo RH et al., Proceedings of the National Academy of Sciences. 1994 Mar. 15; 91 (6): 2076-80). Although early studies have indicated some promise in this approach (Laske DW et al., Nature medicine. 1997 December; 3 (12): 1362-8; Rand RW et al., Clinical Cancer Research. 2000 Jun. 1; 6 (6): 2157-65; Weber FW et al., InLocal Therapies for Glioma Present Status and Future Developments 2003 (pp. 93-103); Lidar Z et al., Journal of neurosurgery. 2004 Mar. 1; 100 (3): 472-9), a Phase III clinical trial (PRECISE) of CED did not show substantially improved outcomes in patients with recurrent glioblastoma when compared to a group of patients treated with the diffusion based BCNU wafer (Kunwar S et al., Neuro-oncology. 2010 Aug. 1; 12 (8): 871-81). One explanation for this result is that the drugs were not delivered in adequate concentrations over the entirety of the tumor as on average, only 20% of the target area was covered by the drug (Sampson JH et al., Journal of neurosurgery. 2010 Aug. 1; 113 (2): 301-9).

Following these early CED trials several design improvements have been made. Reflux of the drug along the insertion tract was a cause of poor drug dispersal, thus limiting the reach to targeted areas, so catheters featuring a step change near the infusion point were developed that decreased incidence of reflux (Krauze MT et al., Journal of neurosurgery. 2005 Nov. 1; 103 (5): 923-9). There is heterogeneity in brain tissue and fluid movement in the brain that can limit drug distribution (Jain RK, JNCI: Journal of the National Cancer Institute. 1990 Jan. 3; 81 (8): 570-6), and another means of improving the volume of dispersed drug is by deploying multiple needles from a single primary cannula. A four-port catheter, designed specifically for CED treatment of brain tumors, has been developed by the Cleveland Clinic (Vogelbaum MA et al., Journal of neurosurgery. 2018 Apr. 13; 130 (2): 476-85). This design has reflux-arresting features and is relatively flexible, with intended use for infusions lasting 48 to 96 hours (Vogelbaum MA et al., Journal of neurosurgery. 2018 Apr. 13; 130 (2): 476-85). It has been used in clinical trials to infuse topotecan and has demonstrated measurable improvements in clinical outcomes (Vogelbaum MA et al., Journal of neurosurgery. 2018 Apr. 13; 130 (2): 476-85). Another multiport catheter design, which was used as a basis for this work, originated with a prototype developed for use with fiberoptic microneedles, able to deliver heat as well as drugs during infusions (Andriani RT, Design and validation of medical devices for photothermally augmented treatments (Doctoral dissertation, Virginia Tech)). The design was further developed to improve repeatability and microneedle deployment characteristics (Elenes EY et al., Exon Publications. 2017 Sep. 20:359-72). The most recent catheter design featured six microneedles, with one microneedle deploying from the tip of the main cannula and five microneedles deploying from the sides (Elenes EY et al., Exon Publications. 2017 Sep. 20:359-72). Tests of this design in cadaveric pig brains have achieved 5.8 times the volumetric coverage of a single port catheter.

Previous studies have suggested the importance of visualization of infusions in order to ensure complete coverage of the tumor (Kunwar S et al., Neuro-oncology. 2010 Aug. 1; 12 (8): 871-81; Sampson JH et al., Journal of neurosurgery. 2010 Aug. 1; 113 (2): 301-9). Magnetic resonance imaging (MRI) allows this, and the contrast agent Gd-DTPA (gadopentetate dimeglumine) has been added to commonly infused CED drugs and has proved a good marker of drug distribution (Mardor Y et al., Cancer research. 2005 Aug. 1; 65 (15): 6858-63). Barua et al was able to saturate 95% of a pontine mass legion using a robot-guided system that utilized serial real-time MRI to visualize the distribution of infusate at the beginning of the infusion and estimate the infusion volume required to saturate the target area (Barua NU et al., Acta neurochirurgica. 2013 August; 155 (8): 1459-65). Additionally, Vogelbaum et al used MRI to confirm positioning of the Cleveland Multiport Catheter, to visualize infusion during the first two hours of infusion and then to monitor infusion volume once a day during a 96 hour infusion (Vogelbaum MA et al., Journal of neurosurgery. 2018 Apr. 13; 130 (2): 476-85). While it is becoming more common to conduct intra-infusion imaging, studies to date have used the imaging to monitor and measure the infusion rather than use it as a tool to correct and optimize it (Sammet S, Abdominal Radiology. 2016 Mar. 1; 41 (3): 444-51). Therefore, with a means of visualizing infusions in real time through MRI, there is an opportunity to precisely manipulate the microneedle positions and make adjustments during an infusion which could allow a clinician to achieve complete coverage of the tumor.

MRI systems present design constraints that must be considered when devising equipment for safe and effective use, as they require very large static magnetic field (>3T in many cases) and radio frequency (RF) radiation to produce images (Sammet S, Abdominal Radiology. 2016 Mar. 1; 41 (3): 444-51). Materials with high magnetic susceptibility are unsuitable because of the force they experience in the magnetic field (Schenck JF, Medical physics. 1996 June; 23 (6): 815-50). Conductors that form closed loops can have a current induced by the RF magnetic field, and this current can heat the conductor (Sammet S, Abdominal Radiology. 2016 Mar. 1; 41 (3): 444-51). Materials with high magnetic susceptibility and the creation of any conductive loops were avoided in the design of the mechanisms and transmission.

The present study a system is designed and tested to position and reposition microneedles within the bore of an MRI with the ultimate goal of improving the drug volume of distribution in CED treatments.

The materials and methods are now described.

The microneedle positioning device consists of four major components: 1) the arborizing catheter, 2) needle deployment mechanisms, 3) transmission system, and 4) drive and control units. The major specifications for the microneedle positioning device are as follows: non-metallic materials; needle positioning precision targeting<1 mm; needle positioning repeatability targeting<1 mm; maximum needle deployment targeting 3.5 cm; needle movement speed targeting 0-2 mm/min (adjustable); and external/remote control.

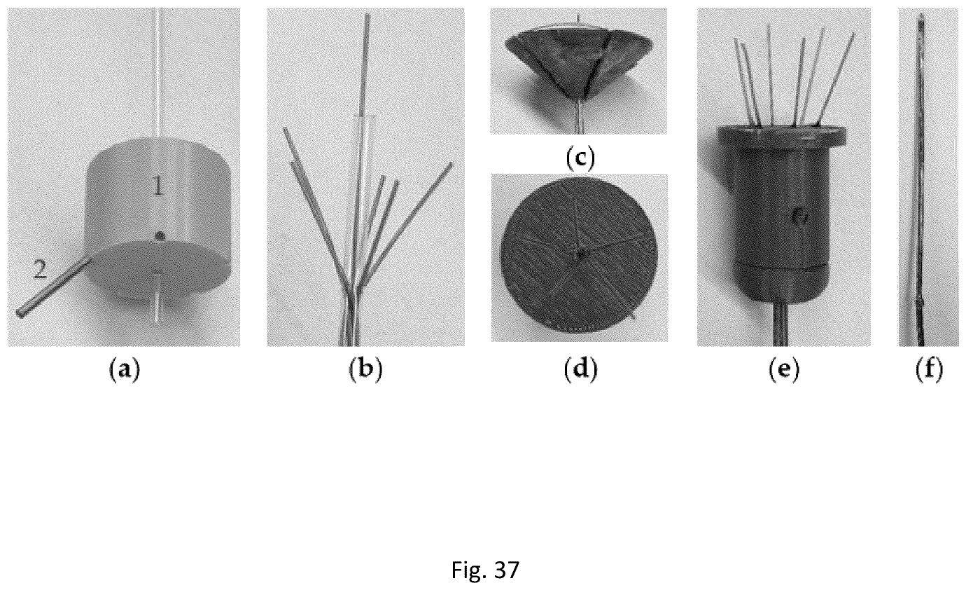

The arborizing multiport catheter design (Elenes EY et al., Exon Publications. 2017 Sep. 20:359-72) was used as the basis for the positioning system. The design featured six microneedles, with one needle deploying from the cannula tip and five needles deploying outwardly from the main cannula at approximately 26 degrees (Elenes EY et al., Exon Publications. 2017 Sep. 20:359-72). The fabrication process and guide tube material were altered from previous work to improve repeatability and to interface with the positioning system. Microneedle guide tubes were made using polyimide tubing (0.51 mm ID, 0.66 mm OD, Nordson Medical P/N 141-0028), the outer shell of the main cannula was made with PEEK tubing (2.74 mm ID, 3.00 mm OD, Nordson Medical P/N 144-033), and medical grade UV cure acrylic adhesive (Loctite AA 3926) was used for adhesive and for forming the cannula tip. A flexible tubing (D.O.T. Hard Nylon Plastic Tubing for Air, Opaque Blue, 5/64″ ID, ⅛″ OD) was used to house the lengths of polyimide guide tube that extended outside the rigid portion of the cannula. The flexible tube added to the end of the cannula allowed freedom to position the main cannula relative to the needle actuation block, which was important given the space limitations in the MRI bore. The fabrication process is shown in FIG. 37.

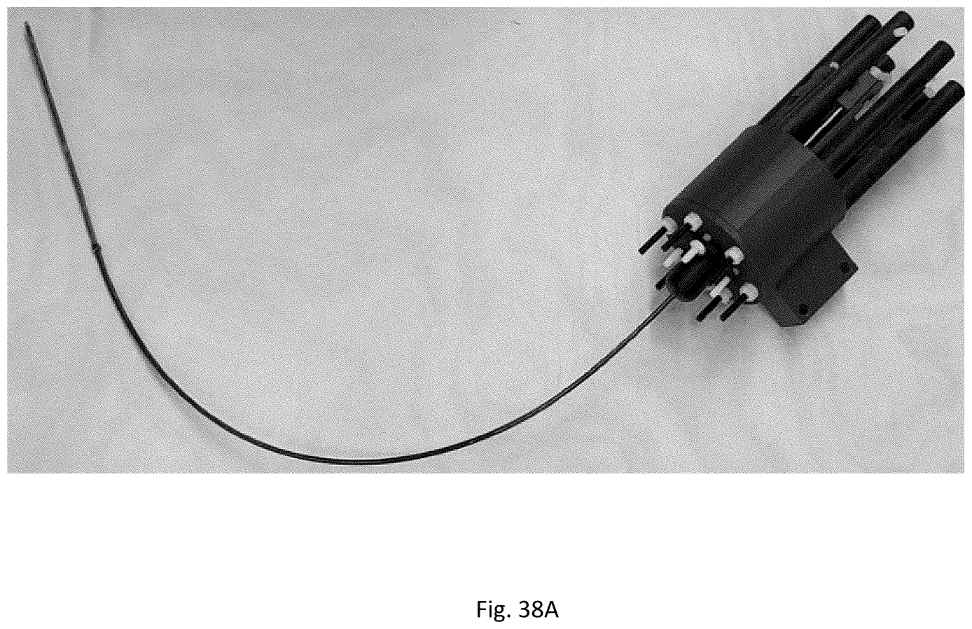

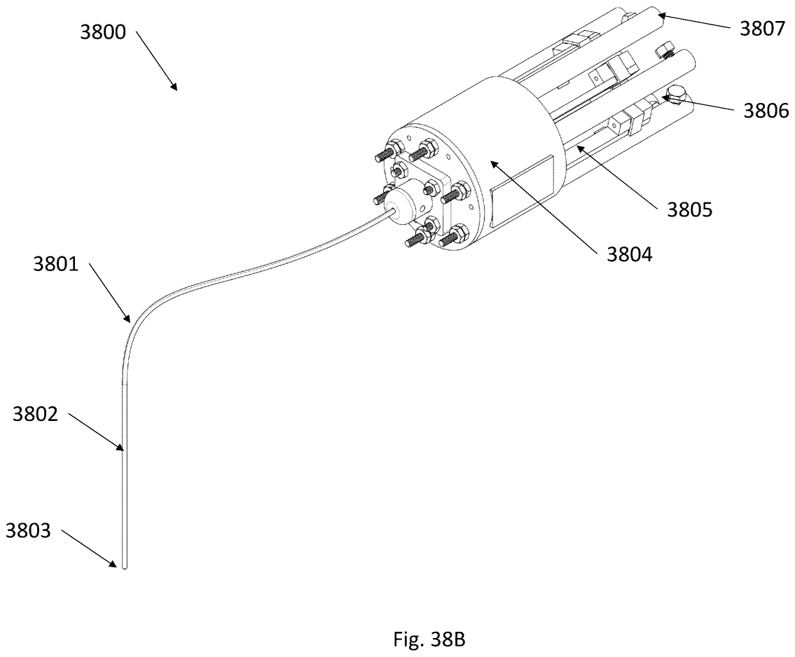

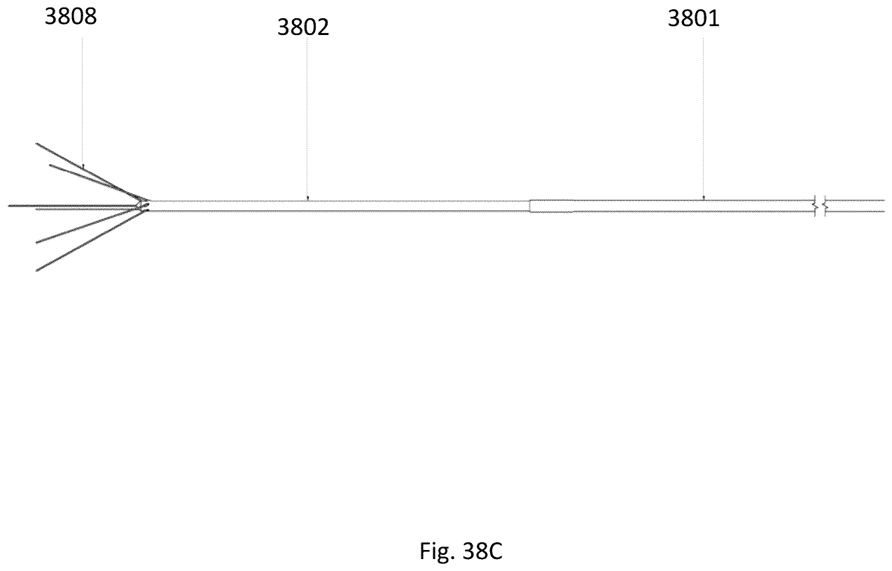

An exemplary complete main cannula attached to an actuating block is shown in FIG. 38A, with a line drawing of the device shown in FIG. 38B. With reference to FIG. 38B, the exemplary system 3800 includes an actuation block 3804, a main cannula connected at the proximal end to the actuation block 3804, and having a flexible portion 3801, a semi-rigid portion 3802, and a distal tip 3803. The actuation block 3804 further comprises one or more microneedle lifters 3805, microneedle holders 3806, and actuation block knobs 3807. FIG. 38C shows a detail view of the end portion of the main cannula, showing the semi-rigid portion 3802 and the flexible portion 3801, with the microneedles 3808 in a deployed or extended position.

Although the depicted embodiment shows six microneedles, it is understood that the system as disclosed herein may comprise more or fewer than six microneedles, including for example one, two, three, four, five, seven, eight, nine, ten, eleven, twelve, or more microneedles.

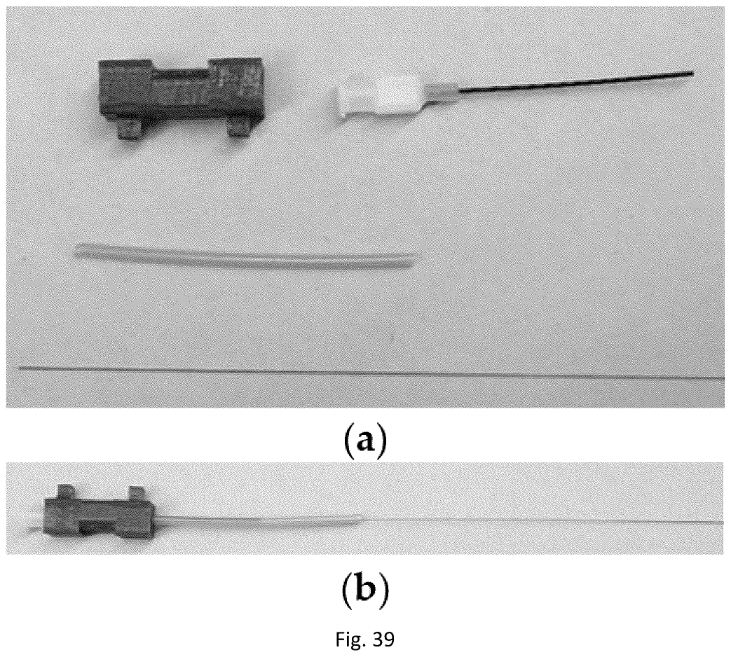

Microneedles were made using flexible fused silica capillary tubing (0.37 mm OD, 0.18 mm ID, TSP180375), a 1.5″ plastic needle with a Luer lock (TECHCON TS22P-1-1/2PK 22 GA), a 3 cm length of PEEK tubing (0.37 mm OD, 0.18 mm ID, TSP180375), and Loctite Ultra Gel Control Super Glue, as shown in FIG. 39. The rigid PEEK tube was needed to push the needle into the angled paths of the actuating block during needle deployment.

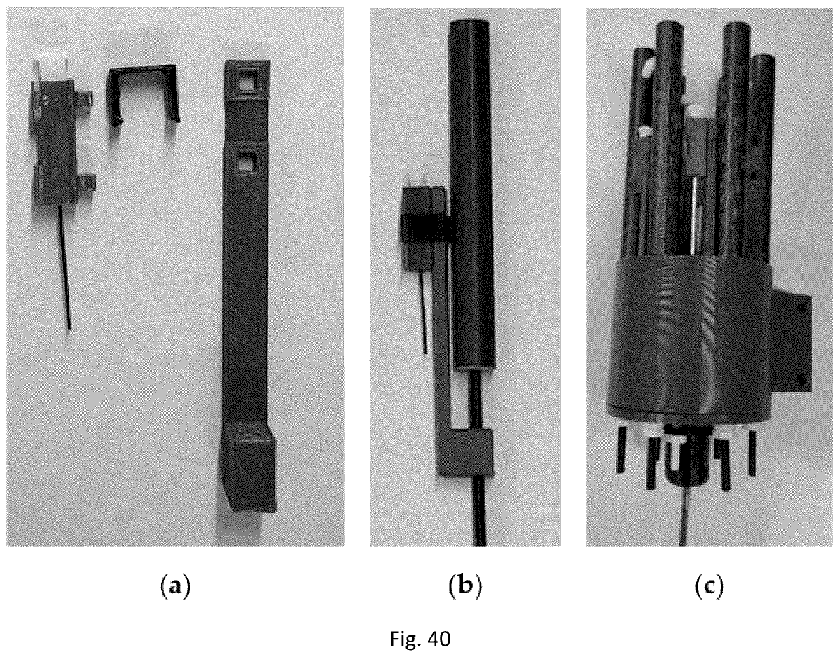

The desired precision of the microneedle movements was less than 1 mm. A simple way to achieve this precision was the use of a lead screw, which converts the angular motion of a rotating screw into the linear motion of a nut. Lead screw mechanisms often use square or Acme type threading, because these experience less friction during operation. This application had such low speed and torque requirements that off-the-shelf nylon threaded rods with standard V-shape threads could be used. An 8-32 nylon rod gave 0.79 mm of linear motion per revolution, which was sufficient for the application. The lift arm, FIG. 40(a), was additively manufactured in PLA and tapped in the bottom portion for operation with the lead screw. Holes at the top of the lift arm provided a secure means of attaching the microneedle, as shown in FIG. 40(b). Attaching the Luer lock end of the microneedles to a lift arm provided the required linear motion.

Six identical microneedle deployment mechanisms were built into a custom housing (FIG. 40(c)). Internal ducts in the housing narrowed the trajectories of the microneedles, guiding the microneedles into the flexible tube of the main cannula. The housing also provided guide shafts for the lift arms, with hollow spaces in the housing forcing the linear motion of the lift arms as the screw turned. The lead screws were glued into a garolite coupling pieces for attachment to the transmission. Six of these lead screw mechanisms were combined in a single actuating block piece, also additively manufactured in PLA.

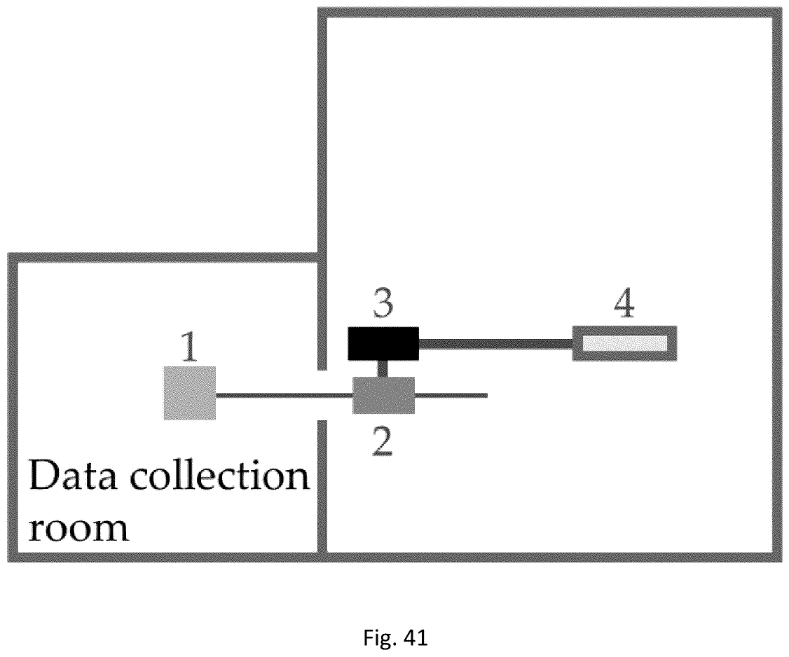

Electromechanical stepper motors were used to provide position input to the microneedle actuation devices. In order to keep these motors a safe distance from the MRI coil, a transmission system of rotating rods was developed, much like the line shafts used prior to the development of electrical power distribution (Devine WD, The Journal of Economic History. 1983 June; 43 (2): 347-72; Casanova F et al., Journal of neuroscience methods. 2014 Nov. 30; 237:79-89). In this configuration, the motors could be housed in the adjacent MR equipment room and the transmission could be routed into the MR Magnet room via a pass-through. In the as tested configuration, the transmission connected to ceiling-height motors, traveled a straight distance of approximately three meters along the ceiling, turned towards the ground, and made a right angle turn towards the actuation devices. A schematic of the system is shown in FIG. 41.

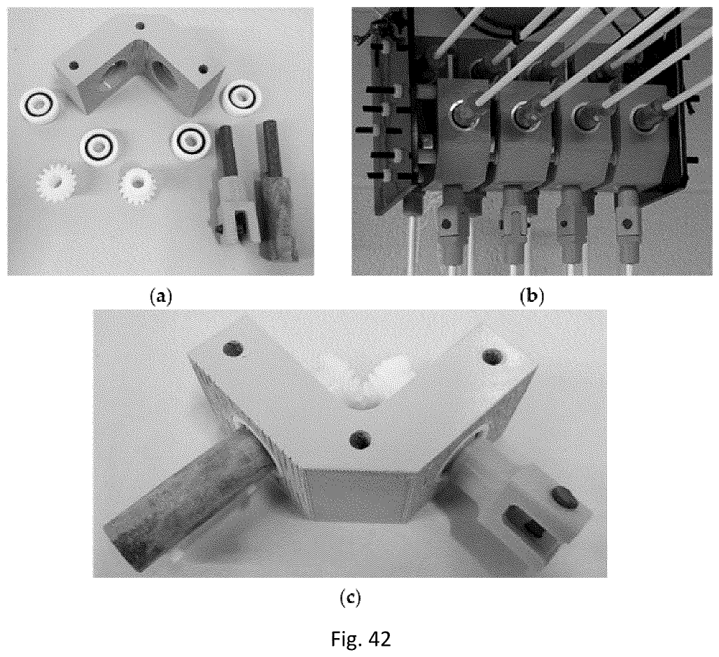

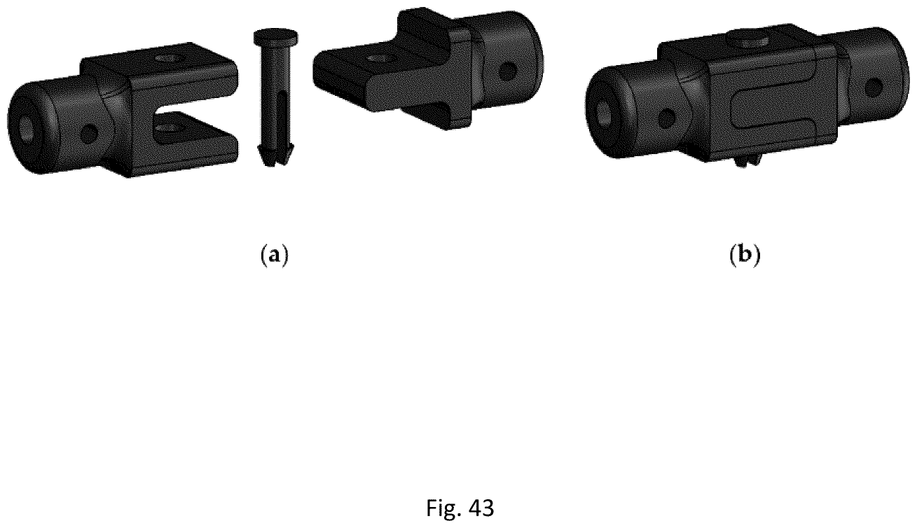

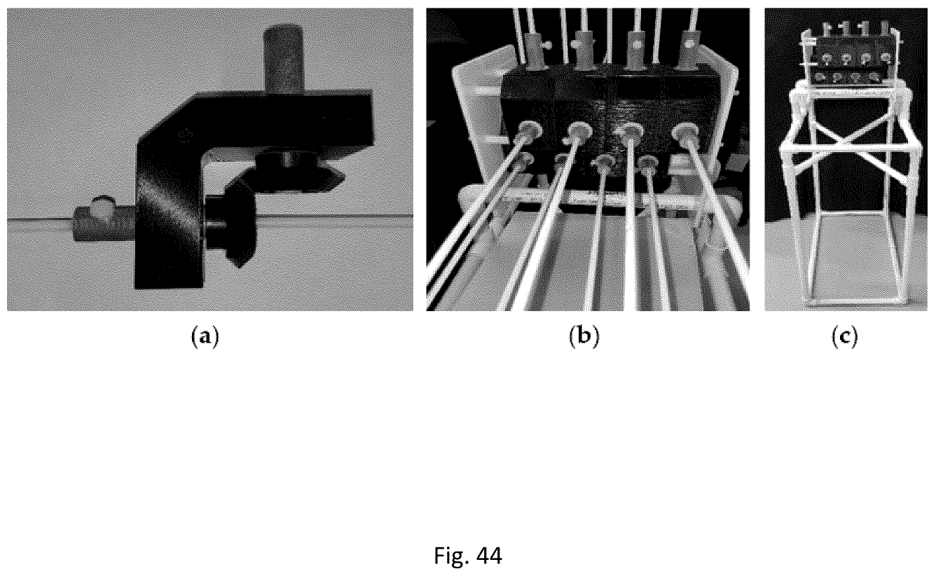

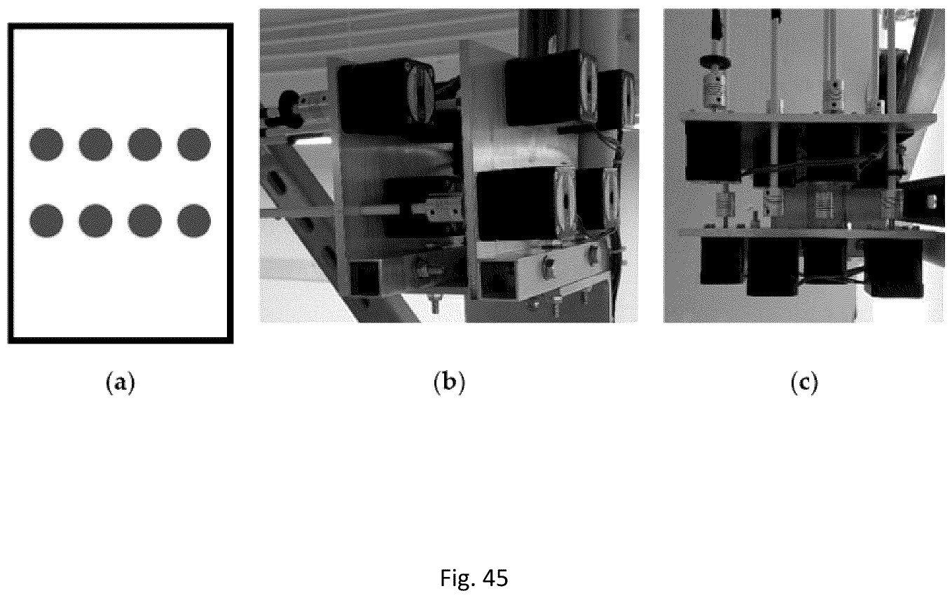

The transmission consisted primarily of rotating ¼″ fiberglass rods. Right angle turns in the transmission were accommodated using gear boxes constructed with nylon miter gears, ⅜″ garolite shafts, and glass ball bearings (⅜″ ID, 1⅛″ OD), as seen in FIG. 42. Single pairs of miter gears were assembled in additively manufactured housings.

It was important that the system be easily removed when not in use. This was made possible by the use of quick connects FIG. 42. The design used a pin with a flared end to hold together the two mated pieces. The mated pieces could be attached to the rotating rods and the shafts of the mechanisms at junctions to allow quick and easy setup of the transmission. The quick connects were utilized at the ceiling-level junction in the transmission.

Not all patients are positioned at exactly the same place in the MRI coil, and because of this the rigidity of the rotating rod transmission system presented a problem. The solution developed was to use hollow shafts in the final right-angle gear box leading to the actuating mechanisms. Set screws could then be tightened on the rotating rods after the actuation mechanisms had been positioned in the MRI scanner. The scanner-level junction boxes that allowed adjustment of rod length were made in a manner similar to the other miter gear junctions. The ¾″ garolite shafts were given a 17/64″ hole through the middle to allow for adjustment of the horizontal fiberglass rods leading to the actuation devices. The shaft pieces were ¾″ outside the bearings to provide sufficient material for a ¼″ nylon set screw. The two rows of miter gear pairs were staggered so the two rows of rods would not interfere with each other. The assembled module was supported by a stand constructed from ¾″ PVC pipe.

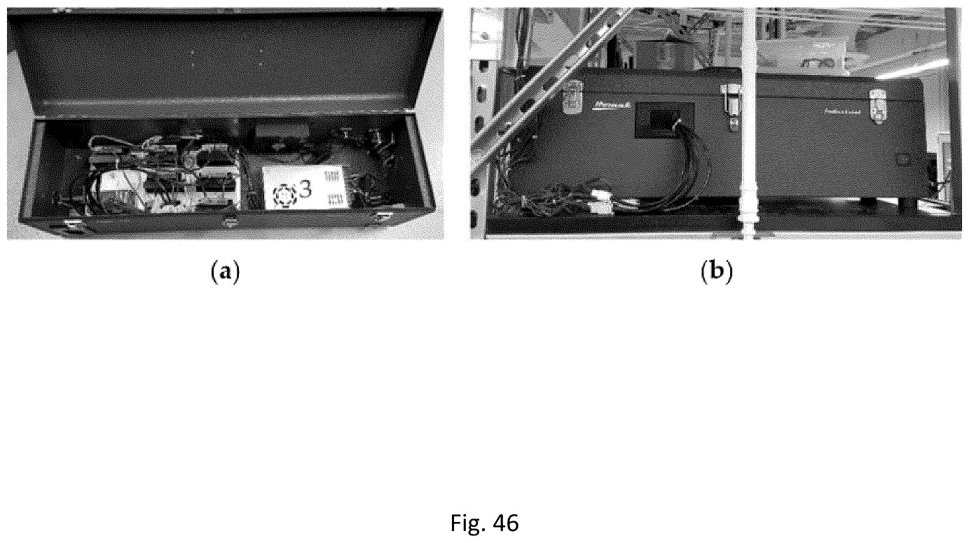

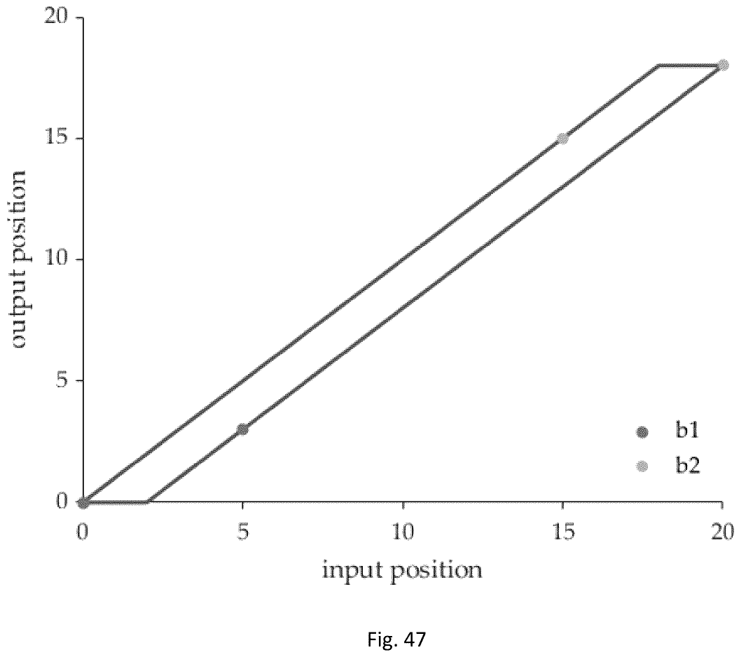

The motors driving the system needed to be capable of precise position control. Speed was not a significant concern for the devices, because needles cause less damage to brain tissue when they move more slowly (Casanova F et al., Journal of neuroscience methods. 2014 Nov. 30; 237:79-89; Greenway B, Industrial Robot: An International Journal. 2000 Aug. 1). Stepper motors were chosen for their low cost and simple implementation. Off-the-shelf NEMA 17 stepper motors (2 amps, 1.4 Ohms, 3mH, 59Ncm) were used. Because the torque requirements were so low, the missing of steps was not a concern. The motors were mounted on a motor bank assembly featuring eight motors mounted on two aluminum plates in a staggered configuration FIG. 46. The eight motors allow extra capability for controlling deployment and rotation of the main cannula. The transmission rods exited the motor bank in two rows of four with spacing of 5 cm between rods.

An Arduino Mega type prototyping board was used for control of the system. This controller provided the many digital output pins needed for control of the multiple stepper motors. The stepper motors were driven using TB6600 stepper motor driver modules. A 12-volt, 300-watt power supply powered the stepper drivers. All components were housed in a custom electronics box. Commands could be given to the controller using the serial monitor of the Arduino IDE (integrated development environment).

Repeatability was the primary metric used to quantify performance of the positioning system. To further characterize the devices, backlash hysteresis type behaviors were also observed. In addition, angles of the microneedle deployments were determined because of their effect on overall system accuracy.

Two of the primary performance metrics related to robotic device positioning are the accuracy and repeatability of movements. Repeatability is how well a robot can return to a position, while accuracy is how well a robot can reach a particular point in space based on a given coordinate system (Mousavi A et al., In2015 3rd RSI international conference on robotics and mechatronics (ICROM) 2015 Oct. 7 (pp. 640-644)). The repeatability formulation in the ISO 9283 standard (“Manipulating Industrial Robots-Performance criteria and related test methods”) was used for calculating repeatability. This standard includes detailed test requirements for things such as load, range of motion, path, and other considerations (Mousavi A et al., In2015 3rd RSI international conference on robotics and mechatronics (ICROM) 2015 Oct. 7 (pp. 640-644)), but much of this was not relevant to this particular application and was not used here.

The ISO 9283 method of calculating positioning repeatability (Rp) from the attained positions (xa) and the average attained positions (x) for n repetitions uses the following equations:

$${l}_{i} = \sqrt{{( {x}_{a i} - \overline{x} )}^{2}} \qquad ( Eq . 17 )$$

$$\overline{l} = \frac{1}{n} {\sum }_{i = 1}^{n} {l}_{i} \qquad ( Eq . 18 )$$

$${S}_{l} = {\sum }_{i = 1}^{n} \sqrt{\frac{( {l}_{i} - \overline{l} )}{n - 1}} \qquad ( Eq . 19 )$$

$${R}_{p} = \overline{l} + 3 {S}_{l} \qquad ( Eq . 20 )$$

This calculation was used to quantify performance in microneedle placement.

Another concern with this system was backlash-type hysteresis effects. Mechanical systems in which a transmission separates a drive from a load will have some backlash (Masia L et al., InRehabilitation Robotics 2018 Jan. 1 (pp. 47-61)), and in this system the positioning devices were far removed from the driving motors. The arborizing catheter positioning system currently operates using open loop control, so knowledge of the backlash characteristics is important for determining how much to adjust input when changing directions. This is especially critical in the case of microneedle retraction, because if the main cannula were to move while a microneedle was not fully retracted there could be serious damage to brain tissue. Backlash-type effects in all the positioning devices and the transmission were examined by observing the position loss through changes in direction.

Backlash is quantified here as the difference between an input magnitude and the resulting output position change during a change in direction. Many cable conduit type mechanisms, which are similar in form to the microneedle deployment mechanism, have asymmetric backlash profiles with non-linear regions around changes in direction (Dinh BK et al., Robotics and Autonomous Systems. 2017 Jun. 1; 92:173-86; Do TN et al., Mechatronics. 2014 Feb. 1; 24 (1): 12-22) In this work possible nonlinearities in the vicinity of the direction changes were not studied. Because the device will not be making frequent direction changes, the position loss over a large movement after a change in direction is of more relevance than the backlash characteristics in the immediate vicinity of the curve. Backlash values were calculated at both direction changes to get an indication of any possible asymmetries.

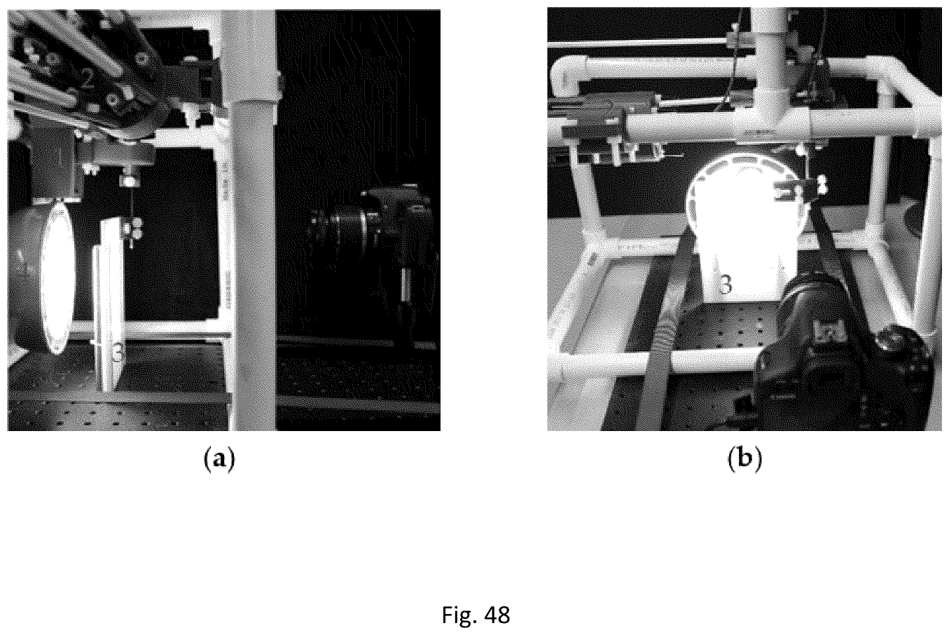

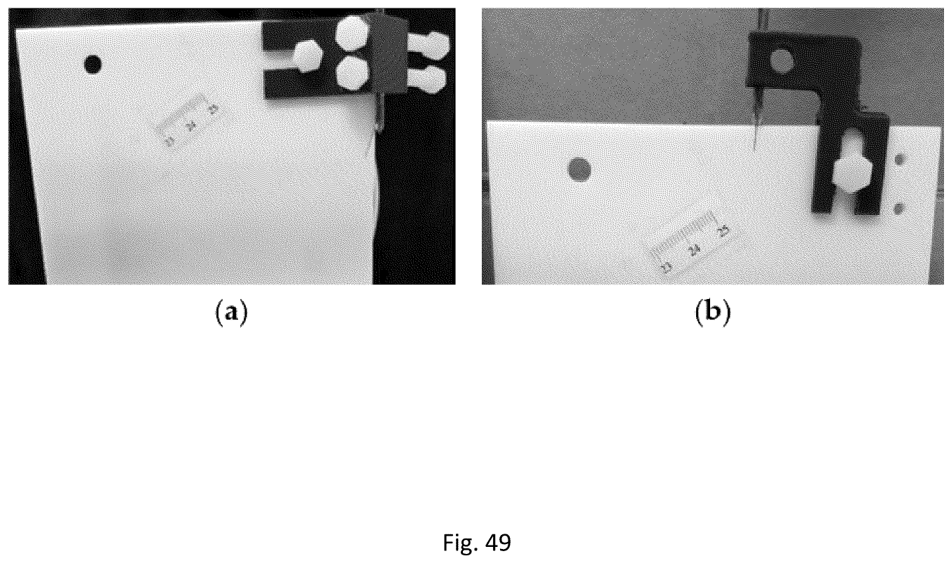

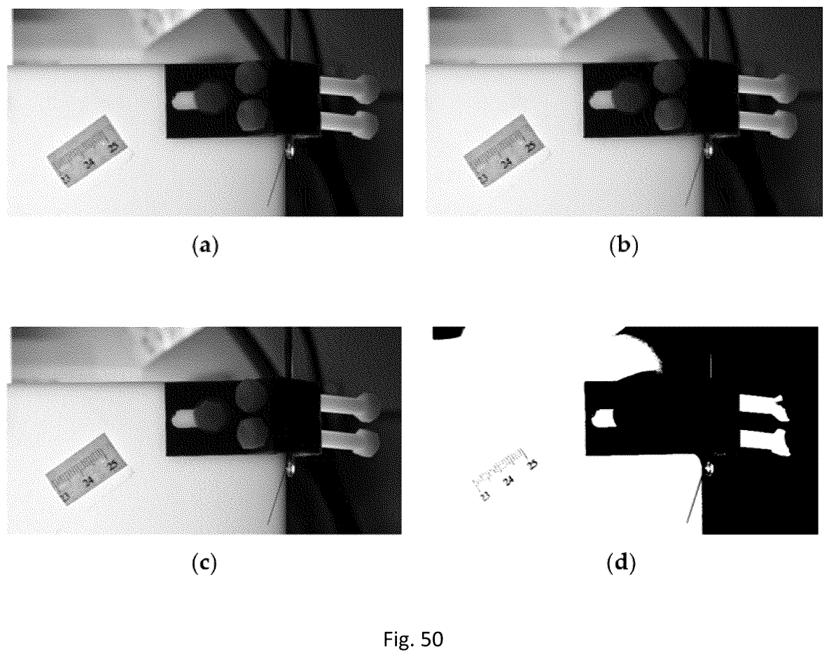

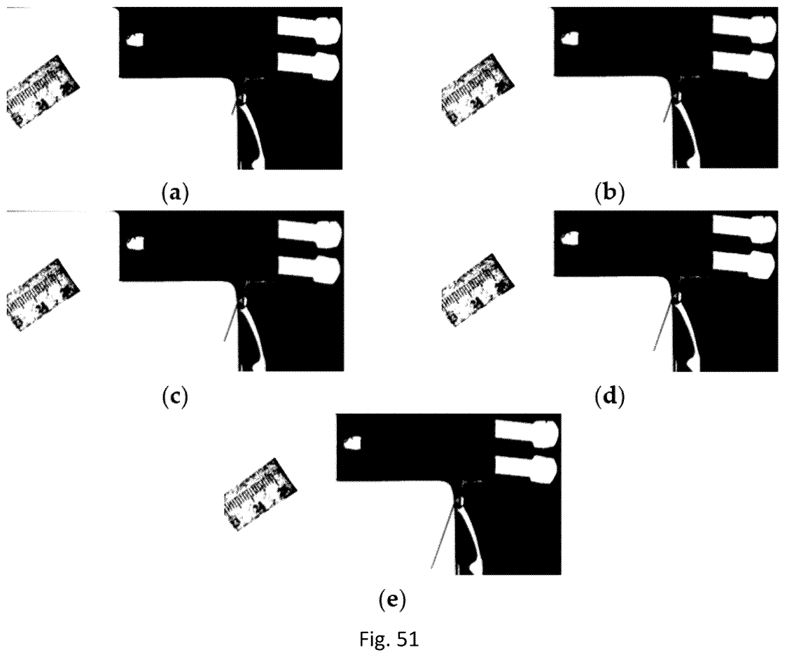

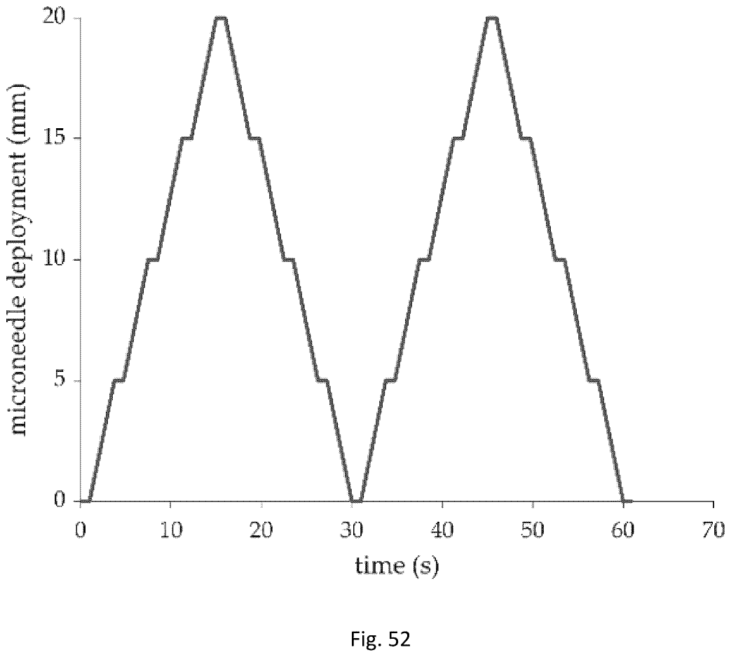

All movements were tested with the arborizing catheter operating in air. For the purposes of testing, this device was attached to a PVC pipe frame FIG. 48. This frame was positioned on a table with an optical board and attached to the transmission for testing of the system. A white acrylic background was mounted to the board 41 mm away from a Canon EOS REBEL Tli/EOS 500D digital camera. A fluorescent lamp provided backlighting for the background to improve contrast in the images. The main cannula was positioned next to the white background and held in place by nylon screws and an additively manufactured clamp, as shown in FIG. 49. The main cannula was oriented such that the microneedle being tested deployed parallel to the acrylic background and as closely to the background as possible without touching it. The microneedle was deployed approximately 5 mm for an initial position that could be seen on the white background. In-plane movements of the individual microneedles were captured using the digital camera to takes pictures after changes in positions. Performance of the transmission was examined at the motor end and actuator end of the transmission using rotary optical encoder wheels and photodetectors.

All photos were taken using the 35 mm focus of the lens. Distortions in the image from the lens were corrected using the “undistortImage” function in MATLAB's computer vision toolbox. The images were then converted to gray scale and then black and white using MATLAB's “rgb2gray” and “imbinarize” functions, respectively. A scale value in pixels was obtained by measuring a 2 cm ruler attached to the acrylic background. The white background was then scanned over the region of interest and the position of the microneedle tip determined.

For examining repeatability, the microneedles were deployed with an input movement of 20 mm in 5-mm increments, and then retracted with an input movement of 20 mm in 5-mm increments. This set of movements was repeated ten times, and pictures were taken during pauses between each 5-mm movement. The input motors rotated at a speed of 2.3 rotations per second for these movements, and this speed was verified using the encoder wheels and photodetectors used for transmission system testing. The first insertion and retraction of the microneedle was ignored to eliminate the unknown state of backlash in the system. The most retracted position of the microneedle after one complete insertion and retraction cycle was then considered as the base point, and displacement of the microneedle tip from this point was used to represent cannula movement. For repeatability testing the most retracted position was analyzed because it would show the least variation from testing noise variables such as gravity effects, touching of the background, and any out of plane motion.

The same 2-cm insertion and retraction sequence of movements used for repeatability testing was also used for examining backlash. Backlash was quantified as the difference between the 5-mm input commands and the actual microneedle movement during direction changes.

One concern was whether the microneedles themselves or the guide tubes from which they deployed were a greater cause of variations in the backlash. This effect was examined by testing the backlash after changing the positions of all the side-deploying microneedles. The center microneedle was not considered because of its very different guide tube trajectory.

The original tests rotated the main cannula so that the microneedle being tested deployed parallel to the acrylic background. This rotation ability is a feature of the device design, and if used with a rotation mechanism could allow the arborizing catheter to obtain an additional degree of freedom. To see what effect twisting the cannula was on backlash, a set of data was collected in which the camera and acrylic background were rotated around the main cannula. All microneedles were tested with the main cannula in the same orientation, testing the effect that twisting the main cannula had in the previous experiments. Microneedles were kept in the same guide tube position in the cannula for both data sets.

It was observed that data varied by more than the standard deviation when a particular test was run again after repositioning. To examine this in more detail, one data set was repeated three times, repositioning the main cannula in the clamp and adjusting the trajectory of the flexible tube of the main cannula between runs.

Optical encoder wheels with twenty holes (and therefore a resolution of approximately 9 degrees) were used to isolate the amount of backlash-type hysteresis and any stick-slip behaviors found in the transmission. These encoders were placed at either end of the transmission lines and the data recorded at both ends of the system. The rods were rotated two rotations in either direction, pausing after every one rotation. This sequence was repeated ten times. An Arduino Mega prototyping board was used to record the times of the pulses from the two sensors, with times recorded at every change in sensor state (rather than only rising or falling state changes).

While not related to the accuracy of any of the tested quantities here, the deployment angles of the microneedles will have a large effect on overall system accuracy. To examine the longitudinal deployment angle off the main cannula, each microneedle was deployed parallel to the acrylic background and pictures were taken. The radial spacing of the microneedle deployments around the main cannula were determined using an image oriented directly at the cannula tip.

The results are now described.

### Microneedle Position Repeatability

Cycles of 2-cm insertions and retractions were repeated 10 times for the repeatability analysis. All five side-deploying microneedles in the cannula as well as the center microneedle were tested. Eq. 17 to Eq. 20 were applied to the data for the most retracted position. Results are as follows: center needle repeatability 0.24 mm; side needle 1 repeatability 0.09 mm; side needle 2 repeatability 0.09 mm; side needle 3 repeatability 0.05 mm; side needle 4 repeatability 0.05 mm; side needle 5 repeatability 0.09 mm.

### Microneedle Backlash

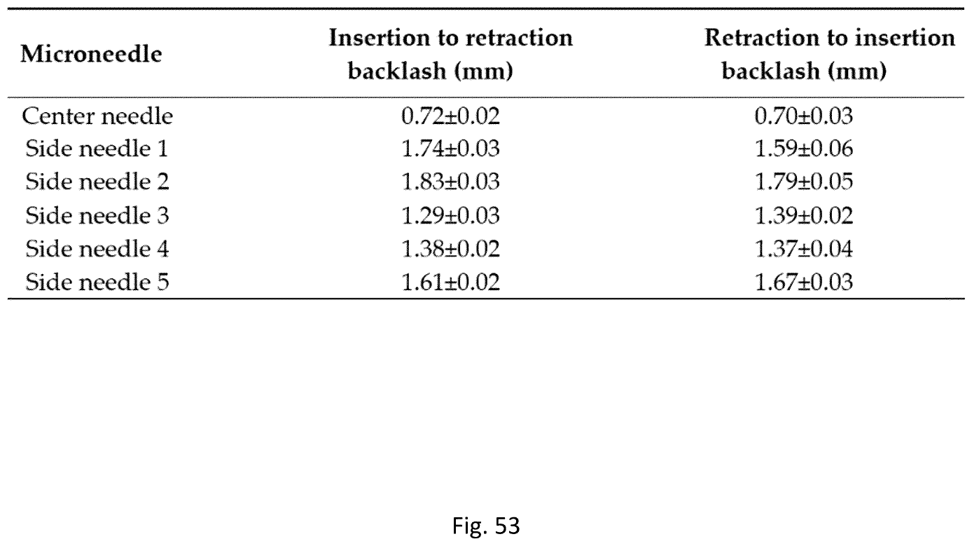

The same insertion and retraction movements used for repeatability analysis were used for backlash analysis. Backlash was quantified as the difference between the 5-mm input commands and the actual microneedle movement during direction changes. Results of backlash tests are shown in FIG. 53. While some microneedles showed greater insertion-to-retraction backlash, others showed less, so no consistent asymmetry was established.

### Particular Microneedle Effect on Backlash

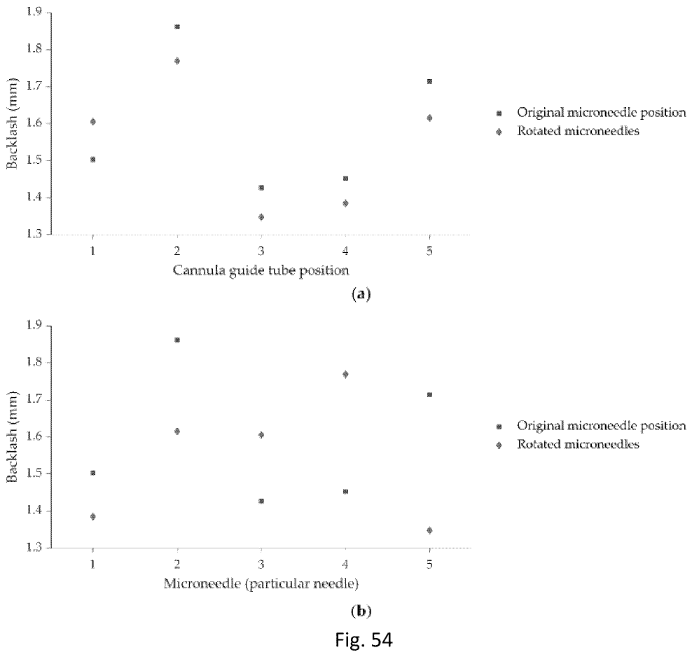

The effect of individual microneedles on backlash was examined by changing the positions of all the side-deploying microneedles. The center microneedle was not considered here because of its very different guide tube trajectory. FIG. 54 shows two different presentations of these results. Because there was no consistent asymmetry, an overall average backlash value is used, rather than separate insertion-to-retraction and retraction-to-insertion values.

The average change caused in the same cannula guide tube position when using a different microneedle (FIG. 54(a)) was 0.09 mm. The average change when using the same microneedle in two different cannula guide tube positions (FIG. 54(b)) was 0.25 mm. This suggests that characteristics of the different guide tube paths had a significantly greater effect on backlash than the particular microneedle used in the path.

### Cannula Rotation Effect on Microneedle Backlash

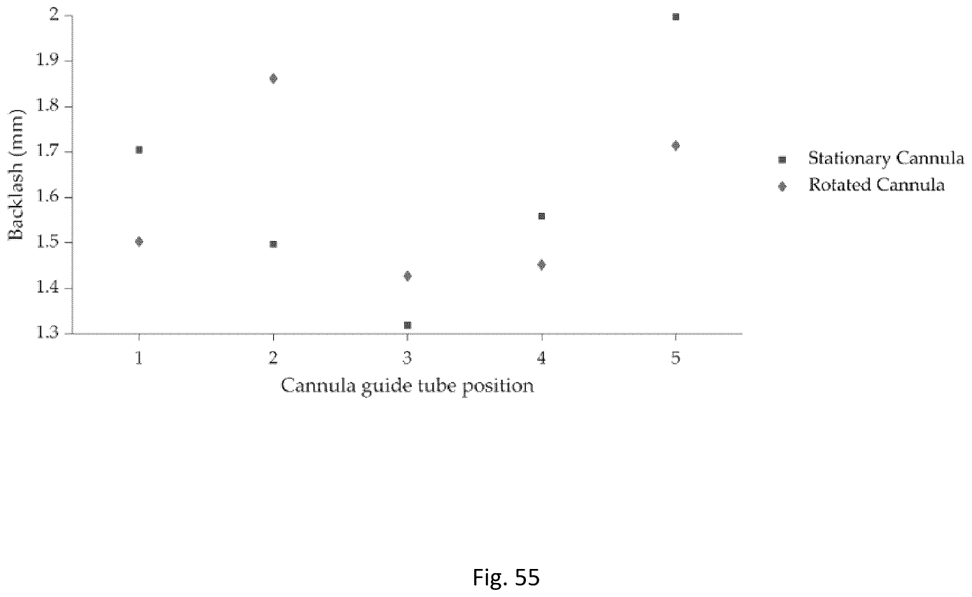

The data set in which the camera was rotated around a stationary cannula was compared to the original data set in which the cannula was rotated to deploy microneedles parallel to the acrylic background. Results (FIG. 55) showed an even split in effect from rotating the cannula, with three guide tube paths having more backlash when the cannula was rotated and three having less.

### Repeatability of Backlash Experiments

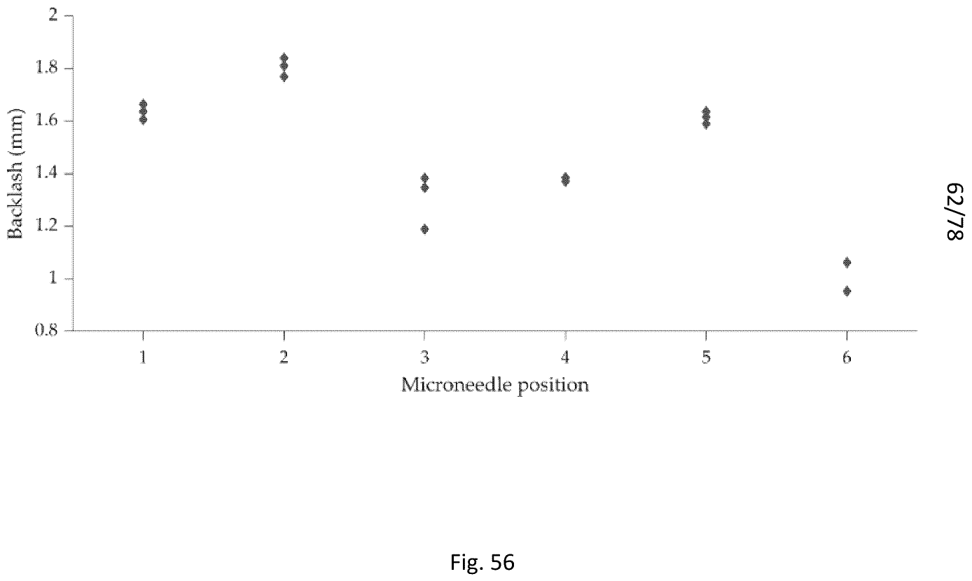

Another consideration was variation in results when a particular experiment was repeated. FIG. 56 shows the results of the third data set (altered microneedle positions, cannula rotated to camera) when the test was repeated three times. The range of backlash values obtained from three repetitions of the test sequence had an average spread of 0.08 mm, with a maximum and minimum of 0.19 mm and 0.01 mm, respectively.

### Microneedle Deployment Angle Effect on Backlash

Variation in microneedle deployment angle was a possible cause of the difference in backlash values between the different guide tube paths in the cannula. The slight difference in microneedle deployment angle likely affected the backlash, but it was unknown whether this contributed substantially to the variation.

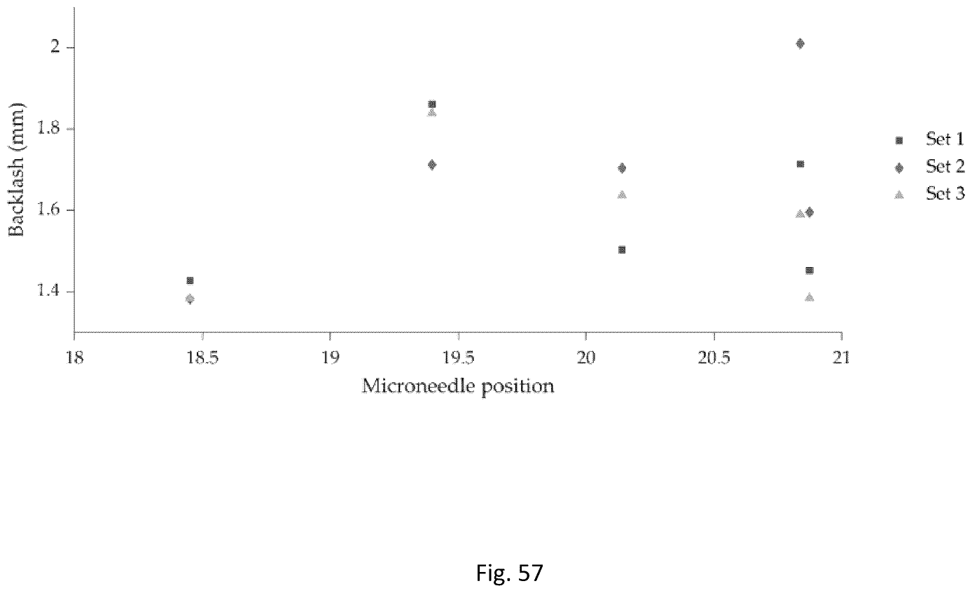

The measured deployment angles of the side-deploying microneedles ranged from 18.5 to 20.9 degrees. FIG. 57 shows the backlash values from the previously used data sets plotted against the microneedle deployment angles. While no conclusions could be drawn about a trend with regard to deployment angle in this limited range, it was apparent that there were differences in the backlash of particular guide tubes that were not related to increased deployment angle.

### Microneedle Deployment Angle

The average microneedle deployment angle was 19.9±1.0 degrees, with a minimum and maximum of 18.5 and 20.9 degrees, respectively. The average separation of microneedle guide tube exits rotationally around the main cannula was 72±11 degrees.

### Transmission System Effects

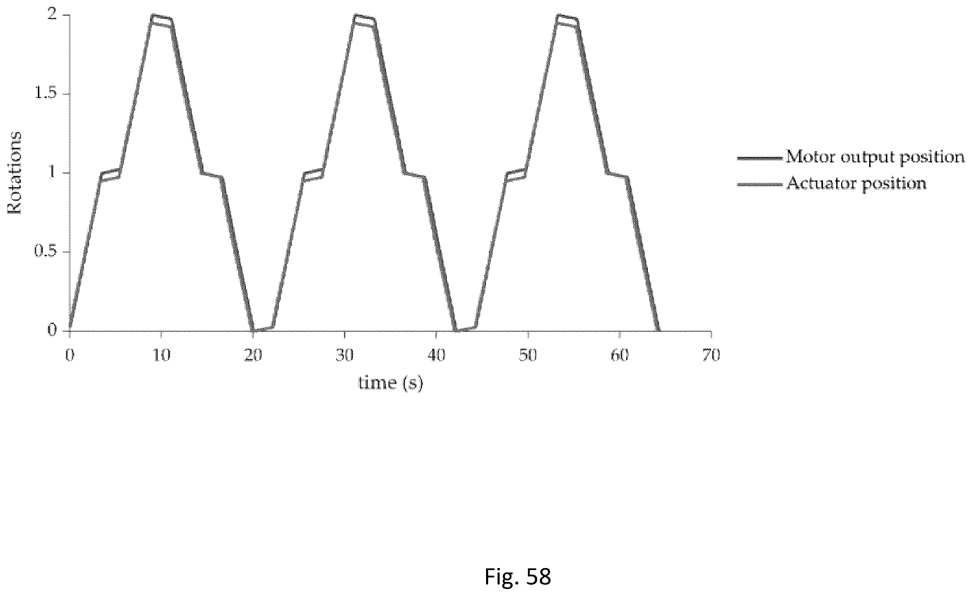

The 9-degree resolution of the optical encoder wheel used for observation was not sufficient to make meaningful measurements of movement repeatability, so only backlash was considered. FIG. 58 shows the first three cycles of tests for microneedle insertion and retraction. The transmission lines leading to the microneedle actuation device showed backlash in the range of 18 to 27 degrees, which would result in an expected microneedle backlash of 40 μm to 60 μm.

The position repeatability for all microneedle deployments was better than the target metric of 1 mm. The worst repeatability, at 0.24 mm, was found in the center microneedle. Somewhat unexpectedly, the microneedle with the worst repeatability was the center microneedle, which had the least backlash. All the side microneedles had repeatability less than 0.1 mm.

The backlash values ranged from 1.29 to 1.82 mm for the side-deploying microneedles. No consistent asymmetry in backlash was shown, with some microneedles showing greater backlash on insertion-to-retraction direction changes, and others on retraction-to-insertion direction changes. The variations in the shape and friction of the individual microneedle paths were such that no asymmetry can be predicted in microneedle deployment backlash.

Backlash values had small standard deviations within a particular sequence of insertions and retractions (all less than 0.07 mm), but backlash between different runs varied substantially. Backlash values in all data sets ranged from 0.78 to 1.1 mm in the center microneedle and from 1.2 to 2.0 mm in the side-deploying microneedles. Further tests showed a variety of causes for this, including the particular microneedle used, the particular guide tube in the catheter, and the twist of the main cannula. All these factors produced unpredictable effects.

The microneedle movement in this device presents a problem very similar to that seen in cable conduit mechanisms, also called Bowden cables or tendon sheath mechanisms. These devices use a cable that slides inside a flexible conduit, and their light weight and flexibility make them common in robotics applications (Do TN et al., Mechatronics. 2014 Feb. 1; 24 (1): 12-22). Backlash in these devices arises from the flexing of the cable inside the conduit; the backlash magnitude is affected by the bend in the cable and the space available in the conduit for the cable to move (Do TN et al., Mechatronics. 2014 Feb. 1; 24 (1): 12-22). The microneedle device is similar, in that the microneedles flex inside the guide tubes, and the bend in the tube leading to the main cannula varies as the main cannula is moved(twisting and insertion). It was therefore not surprising that the backlash in the microneedle changed with the twisting and adjustment of the cannula.

During data collection, it was noted that microneedles did not always move smoothly, but instead, tended to “jump”. This “jumping” is likely the result of stick-slip which is caused by friction in a system (Dinh BK et al., Robotics and Autonomous Systems. 2017 Jun. 1; 92:173-86). The smallest theoretical possible input movement from the stepper motors is 1/6400 of a revolution, based on 200 steps per revolution and 32 microsteps per step. If this movement were perfectly transmitted to the microneedle position output, it would result in a 124-nm movement in the microneedles. Visual observation indicated that the output movements were not that smooth. There was noticeable jumping in microneedle movements, which involved points of friction connected to an elastic element. Very slow insertions and retractions were done to try to examine this stick-slip behavior, but all the stick-slip effects were too small to be quantified with the resolution of the available measurement techniques. While some deviation from a perfectly smooth deployment does appear to occur, particularly with the side-deploying microneedle, no quantifiable minimum deployment can be determined given the resolution of the data. It is likely that the jumps caused by sticking and slipping have magnitudes somewhere in the range of the 0.043-mm width of a pixel. Testing with a measurement technique that had better resolution would be needed to verify this. Importantly, these effects are far less than other sources of uncertainty, such as the variation in backlash values, so these jumps would not be a limiting factor in predicting position.

Backlash from the transmission was not expected to be a major cause of microneedle deployment backlash, and this was confirmed by the transmission testing. The backlash in the transmission of the microneedle deployment device, if it were the 27 degrees at the high end of the measured range, would only account for 0.06 mm of backlash in the microneedle output movement. This represents only 3 to 8 percent of the total microneedle backlash. With this very minor contribution to overall backlash, it can also be inferred that any variations in transmission line backlash were not significant causes of differences in microneedle deployment backlash. Backlash in the transmission system could be attributed to compliant twisting of the lengthy fiberglass rods and to gear backlash, which is caused by space in the meshing of the gears. Testing of the transmission was primarily done to verify whether or not the transmission contributed substantially to device performance. No repeatability deficiencies or stick-slip effects could be detected, indicating that both were less than 9 degrees. The transmission had only a minor, if any, effect on backlash.

There was substantial variation in the microneedle deployment angles. This variation likely stemmed from two particular steps in the manufacturing process: forming the holes in the PEEK tube outer wall of the cannula or use of the jig to hold the guide tubes in place while the acrylic adhesive was cured in the PEEK tube. Any slight deviations from perfect alignment during these steps could have had effects on the trajectory of the microneedle guide paths. An additional problem with the jig used for holding the microneedles was that the guide path tube was only constrained on one side. There was no constraint on the position of guide tubes inside the outer PEEK tube of the main cannula. This allowed variations in the trajectories that could not be fully corrected by manipulating the guide tubes from outside the PEEK tube.

Variations in the angles of the microneedle deployment are not necessarily a limitation in the overall accuracy of the system. If the deployment angles of the guide tubes in a particular arborizing catheter were characterized prior to use in a patient, any variations could be accounted for so as not to contribute to error in the positioning. Additionally, backlash compensation values could be adjusted for individual microneedles. This would be advantageous because the variations in backlash between guide tubes were far greater than the variations among tests of any particular guide tube. While this pre-use characterization of an individual cannula would certainly not be an ideal situation for a device in production, it could substantially improve accuracy of the device during any clinical use in the near term.

Another possible way to account for variations in the microneedle deployment between particular guide tubes in a main cannula would be to adjust parameters following an initial insertion. An accurate model of a particular arborizing catheter could be made based on the MR image of the device during its first deployment in a patient. If a repositioning and additional deployment of the microneedles were required, there would be added accuracy in the subsequent positioning.

In this work, layout of the space used for the testing of the needle positioning system necessitated right angled bends in the transmission system, as shown in FIG. 41. This is the most simple construction of the transmission system; however, constraints in other spaces may make right angle bends infeasible and bends of differing angles may be required. This could be accommodated by using off-the-shelf plastic universal joints, and while these components were tested successfully, they were not needed in the system built. The tests performed were generally able to characterize performance of the system, but there were limitations. One was the two-dimensional data acquisition method used. This added noise variables, as in the twisting of the main cannula to align microneedle deployment. Additionally, the tests were performed in air rather than brain tissue or an agarose gel phantom. Further testing should take place in an MRI scanner so that both these deficiencies can be addressed. The tests could be performed in agarose gel phantoms to determine the effects of backlash and repeatability. Operation in an MRI scanner would also allow a test of overall system accuracy, and ultimately when this device is used in a brain that will be the most important quality.

The testing of the microneedle deployment device confirmed the feasibility of the mechanisms used, exceeding the 1 mm repeatability targets in the initial specifications. The long, flexible tube leading to the main cannula allowed freedom to position the arborizing catheter relative to the deployment devices, which is advantageous in the limited space of an MRI bore. This design achieved position repeatability within the target specification. More work is needed to improve repeatability in the main cannula manufacturing process and create more consistent guide tube trajectories.

The rotating rod transmission system proved to be a simple but effective MRI-compatible means of transmitting power to the positioning devices. The transmission was reliable and did not malfunction during any of the testing. The transmission did not have a significant effect on performance of any of the devices. Additionally, because speed was not critical for these devices, the output from the transmission to the devices could be geared down to further isolate the devices from any transmission backlash.

### Example 4-Using Imaging as Feedback for Device Parameters

### Significance

Malignant tumors of the central nervous system are the third leading cause of cancer-related deaths in adolescents and young adults (R. Siegel, et al., CA: A Cancer Journal for Clinicians, 2013) and the leading cause of death in children. Survival rates for younger patients (0-19 yrs) are more favorable than adults at a rate of 65%. However, survival rates can be less than 5% for the elderly (aged 75+yrs), suffering from aggressive malignant gliomas (MGs) such as glioblastoma. Treatment involves surgery, radiation therapy, various chemotherapeutic regimens, and/or combinations of these three modalities. Neither single nor multimodality treatments are curative. At present, treatment of both primary and secondary brain tumors is provided to improve or sustain neurological function of the patient, to diminish tumor growth intracranially, and to lengthen intervals between treatments. Reportedly, mean patient survival is ~15 months and can be prolonged for only a few months in only 26% of cases with adjunctive therapies such as radiation combined with temozolomide. These statistics have not changed satisfactorily in decades despite intense medical research focused on improving treatment. One of the reasons for poor survival is that MG cells typically infiltrate up to 2 cm beyond the volume of primary tumor (F. H. Hochberg et al., Neurology, 1980), making them difficult to detect and treat. These distant, infiltrating cells may be a key factor in tumor progression and resistance to therapy. Systemic treatment of MGs is also limited by insufficient delivery of drugs due to the blood-brain barrier (BBB) and blood brain tumor barrier (BBTB).

Convection-enhanced delivery (CED), pioneered at the NIH/NINDS, has emerged as a promising method for the delivery of high local concentrations of macromolecules to larger regions of brain tissue (P. F. Morrison, et al., American Journal of Physiology-Regulatory, Integrative and Comparative Physiology, 1994). The principle of CED involves the stereotactically-guided insertion of a small-caliber catheter into the brain. Through this catheter, drugs are pumped into the brain parenchyma and pushed primarily through the interstitial space. The infusion can be continued for up to several weeks. Experiments have shown that CED can deliver high-molecular-weight proteins (as large as immunotoxins and radioisotope-conjugated antibodies) 2 cm into the brain parenchyma after 2 hrs of continuous infusion, an order of magnitude higher than the distances obtained with simple diffusion. Moreover, CED did not cause cerebral edema and was unaffected by capillary loss or metabolism of the macromolecule (W. A. Vandergrift, et al., Neurosurgical Focus, 2006). With these initial experiments, CED was established as a viable method for providing regional distribution of large molecules, like proteins and some conventional chemotherapeutic agents, in the brain. Compared with other therapies, CED minimizes systemic and central nervous system toxicity by

local delivery of high concentrations of therapeutics directly to brain tissue.

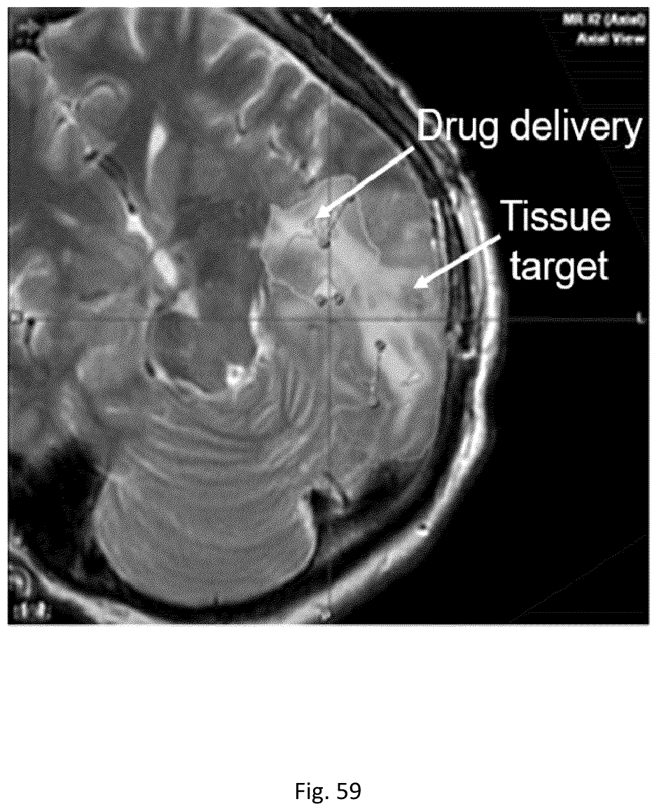

Although CED is more effective than diffusion-based therapies and allows wide distribution of macromolecules, it amounted to inconsistent improvement in patient survival during Phase III clinical trials (PRECISE Trial-S. Kunwar et al., Neuro-Oncology, 2010). A retrospective study, aimed at understanding the reason behind not achieving the company's chosen clinical end-point, concluded that the therapy was limited by poor drug dispersion and estimated that on average only 20% of the tumor margins were covered with the drug J. H. Sampson et al., Journal of Neurosurgery, 2010) (FIG. 59). A primary reason for the poor drug dispersion in the clinical trial was that investigators performed the CED treatment using available catheters designed for different medical applications like direct delivery of liquid content to vessels. Similarly, other CED studies have been limited by the “off-label-use” of various catheters (see Table 1 below) that may not possess the capability to effectively perfuse drugs over large tissue volumes, including margins beyond the primary enhancing tumor detected by magnetic resonance imaging (MRI), that contain infiltrative malignant cells responsible for regrowth of the tumor. In order to achieve targeted delivery and infuse greater tumor volumes, CED often requires insertions of multiple catheters; thus, potentially increasing the risk of trauma to healthy neurological tissue and increasing the probability of seeding the needle tract with cancer cells. Additional drawbacks of CED using one-port catheters listed in Table 1 include clogging of the catheter and reflux of drug along the insertion tract, both of which result in ineffective drug distribution and premature termination of the CED therapy. Significant complications associated with CED catheters have not been clinically realized when using new generation reflux preventing catheters (RPC) of the present disclosure or others despite insertion of multiple catheters. The anatomical heterogeneity of the brain and tumor tissue; differences in permeability between white and gray matter; and issues arising from low-pressure “sinks”, such as cerebrospinal fluid spaces, are all challenges for CED perfusion. Finally, CED efficacy is limited by the difficulties in accurately positioning the catheter and continuously monitoring and controlling drug dispersion. Although MR-compatible stereotactic frames are commonly used for catheter positioning, there are no known catheter systems which can be easily and remotely controlled from the MRI control suite during the CED procedure. The disclosed “Convection-Enhanced Thermo-therapy Catheter System” (CETCS) addresses the above-mentioned clinical needs.

TABLE 1
Examples of catheters used in CED studies

| Type | Benchmark catheters |
|---|---|
| One port | 1. Custom intraparenchymal; Image-Guided Neurologics |
| | 2. 22014 Standard peritoneal catheter; Medtronic |
| | 3. 43209 Cardiac peritoneal; Medtronic |
| | 4. CD 435 end-port; Phoenix Biomedical |
| | 5. Vygon catheter; Vygon US LLC |
| Multiport | 1. Custom fish-mouth; Phoenix biomedical |
| | 2. PS Medical cardiac/peritoneal catheter; Medtronic |
| | 3. Cleveland Multiport Catheter, Infuseon Therapeutics |
| | 4. Neuroinfuse, Renishaw |
| Balloon-tipped | 1. B. Braun Vascular Systems |
| | 2. PowerCross ™ Dialation Catheter; Covidien |
| Hollow-fiber | 1. Twin Star Medical, Inc |
| Reflux- | 1. In-house design; Fiandaca et al. (2008) |
| preventing | 2. In-house design; Krauze et al. (2005) |
| (RPC) | 3. SmartFlow cannula; Clearpoint Neuro |

### Device

Unique features of the disclosed CETCS with clinical relevance include: 1) a catheter comprising arborizing fiberoptic microneedles to broadly distribute drug over large margins to include treatment of the primary tumor and peritumoral infiltrative cells, 2) co-localized, simultaneous photothermal activation(thermo-therapy) to modulate tissue permeability and interstitial fluid pressure (IFP) and enhance drug perfusion to completely saturate target tissue, 3) a remote control system to allow adaptive microneedle positioning (i.e. continuous retraction), drug perfusion via flow or pressure control, pressure sensing, and light delivery, and 4) a computational framework (finite element model) that accurately predicts experimental results.

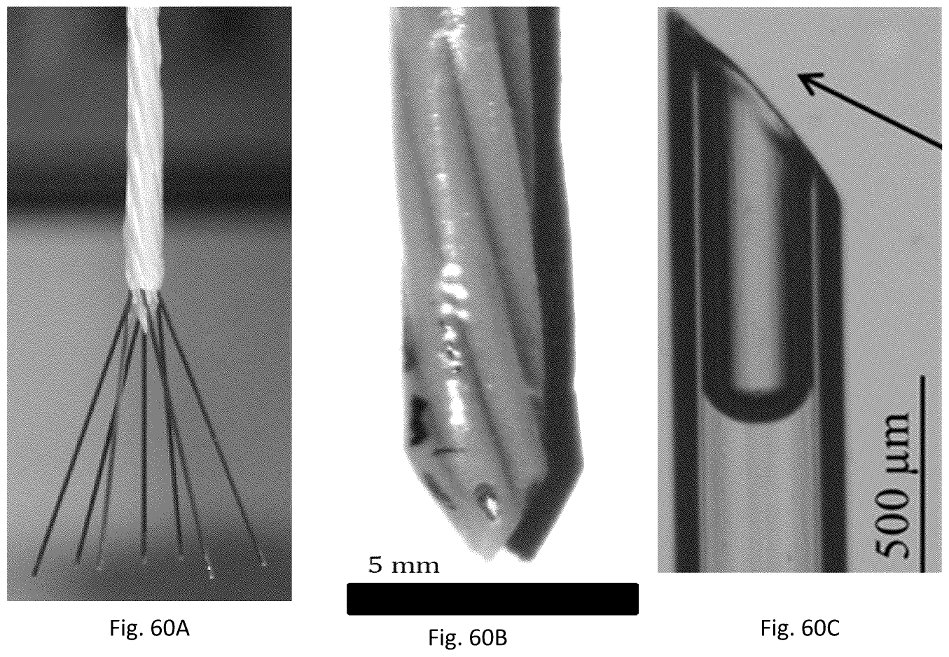

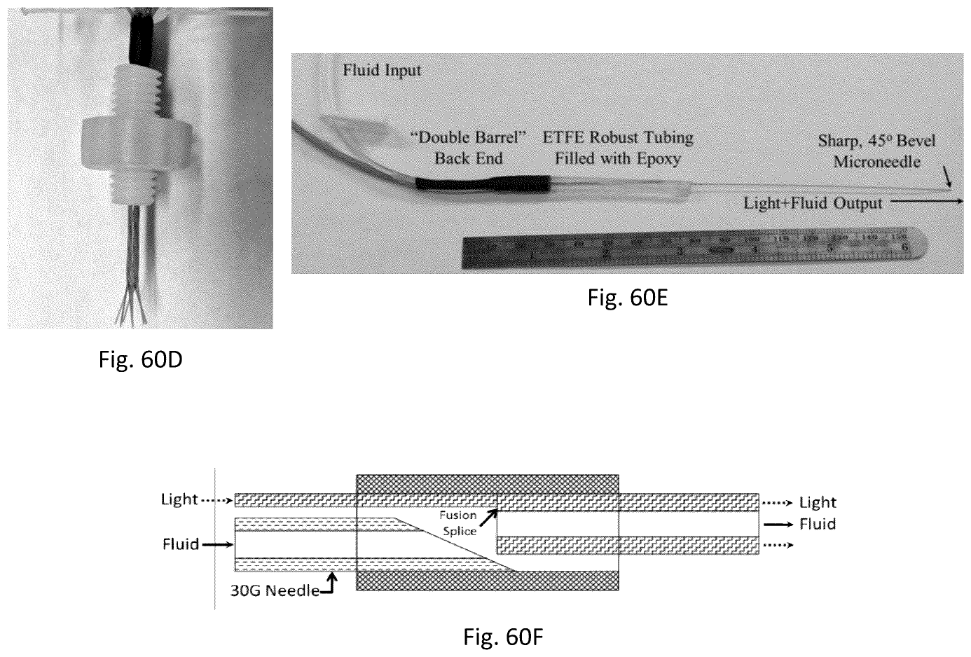

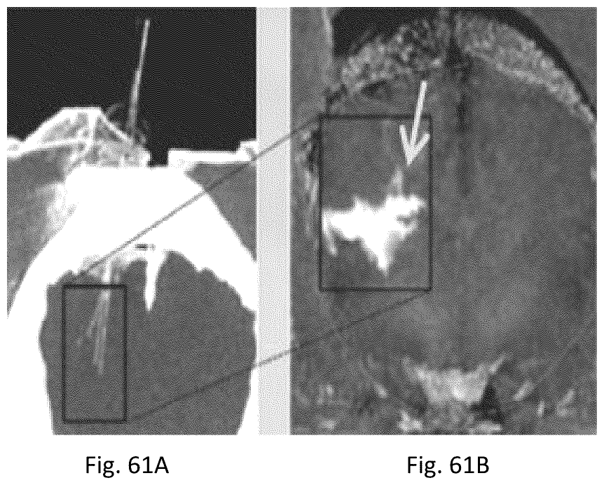

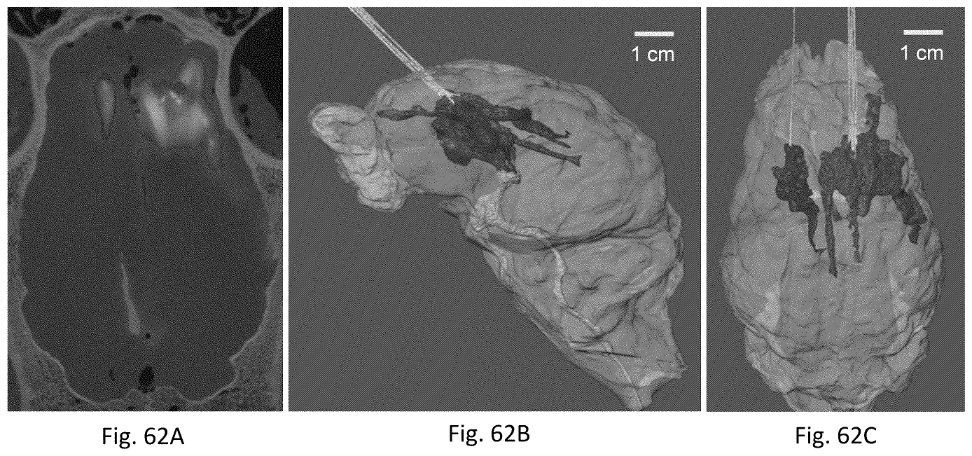

Arborizing catheter—The catheter is comprised of a bundle of 6 microneedles made from hollow optical fibers, which are passed through a rigid cannula and individually arborize (branch-out), penetrating several centimeters into tissues (FIG. 60A-FIG. 60F) (E. Elenes et al., Glioblastoma, Brisbane, Australia: Codon Publications, 2017). Prototypes for the arborizing catheter (FIG. 60A and FIG. 60B) include a cannula (3 mm OD) that is custom-manufactured by spiral twisting biocompatible polyether ether ketone (PEEK) tubing and bonding with epoxy. The distal end of the cannula is polished to a smooth conical tip. The cannula houses seven slender, flexible hollow-silica fiberoptic microneedles (365 μm OD; 150 μm ID) polished to a smooth bevel tip (FIG. 60C). When deploying the microneedles, the twisting of the PEEK tubing allows the needles to branch (arborize) at an angle of up to 30° (angle of peripheral needles from cannula axis) to target clinically relevant tumor volumes and a surrounding margin of 2 cm or more. The catheter can be inserted through a cranial implantable probe guide pedestal (PGP) that is cemented on the skull of the subject (see e.g. FIG. 60D). Once the catheter is inserted, individual microneedles can be deployed independently from the single cannula, providing multiple infusion tracts per one primary cannula insertion tract. The small diameter of the disclosed microneedles can minimize trauma to the brain. Additionally, microneedles can be fully retracted back into the cannula upon completion of the therapy; thus, the tumor-contacting surfaces of needles remains completely within the primary cannula upon removal, thereby reducing the probability of tumor cell-seeding in healthy brain tissue and preventing mechanical damage to the surrounding tissue when extracting the catheter. Because the disclosed device provides direct, local and regional delivery to the brain, it is not necessary to destabilize the BBB, nor deliver high systemic doses of drugs to achieve desired concentrations of drug in the brain. Two major technical challenges inherent in CED are reflux of the infusate up the exterior of the catheter, and clogging. Reflux of the infusate along the needle tract inhibits forward flow into the brain and increases error in measurement of the infusion volume. RPCs mitigate reflux with a designed step change in diameter (e.g., SmartFlow cannula by Clearpoint Neuro) (M. A. Vogelbaum et al., Neuro-Oncology, 2015). The disclosed arborizing catheter design introduces a step change at the cannula-microneedle interface. In previous studies (FIG. 61A, FIG. 61B, and FIG. 62A-FIG. 62C), any reflux was mostly contained within the targeted volume and arrested when reaching the larger body of the cannula. To minimize the effects due to clogging, the disclosed design incorporates continuous retraction, which helps de-core clogged needles and pressure sensors attached to each microneedle. In the event of clogging, indicated by a spike in pressure, which surpasses a predetermined threshold, flow for that specific microneedle can be terminated with continuation of the treatment using the remaining microneedles.

In recent studies (see e.g. FIG. 62A-FIG. 62C) E. Y. Elenes et al., Journal of Engineering and Science in Medical Diagnostics and Therapy, 2020), the arborizing catheter, comprising six infusion ports, was compared to a reflux-preventing single-port catheter. Infusions of iohexol at a flow rate of 1 μL/min/microneedle were performed, using the arborizing catheter on one hemisphere and a single-port catheter on the contralateral hemisphere of excised pig brains. The volume dispersed (Va) of the contrast agent was quantified for each catheter using standard techniques (G. P. Mazzara, et al., International Journal of Radiation Oncology Biology Physics, 2004; and M. F. Dempsey, et al., American Journal of Neuroradiology, 2005). V_{d} for the arborizing catheter was approximately 6-times higher than for the single-port catheter, 2235.8±569.7 mm³ and 382.2+243.0 mm³, respectively (n=7). Minimal reflux of the infusate was observed. With simultaneous infusion using multiple ports of the arborizing catheter, high V_{d} was achieved at a low infusion rate. Thus, the arborizing catheter promises a highly desirable large volume of distribution of drugs delivered to the brain for the purpose of treating brain tumors.

The disclosed devices may in some embodiments be used in conjunction with fiberoptic microneedles, for example using techniques disclosed in U.S. Pat. No. 8,798,722, issued on Aug. 5, 2014 and U.S. Pat. No. 10,220,124, issued on Mar. 5, 2019, both incorporated herein by reference. The contemplated fiberoptic microneedles are in some embodiments capable of delivering both fluids and light simultaneously (see FIG. 60E). The procedure involves fusion-splicing a solid multimode fiberoptic to the light-guiding annular wall of the microneedles (see FIG. 60F). The splice region is subsequently encased within a closed fluid system with a Luer-Lock connector for fluid input. Thus, each microneedle can deliver both fluid and light energy simultaneously. Successful co-delivery of infusates and 1064 nm laser light in both excised and in vivo brain tissue has been shown (R. L. Hood, et al., Lasers in Surgery and Medicine, March 2013; R. L. Hood, et al., Lasers in Surgery and Medicine, September 2013; R. L. Hood, et al., Engineering, 2015). Tumor tissue and normal white matter is inherently more challenging to perfuse by CED due to its lower hydraulic conductivity and higher IFP (~20 mmHg for tumor compared to ~2 mmHg for normal brain). The fiberoptic microneedles allow simultaneous, co-delivery of laser energy and therapeutics to enhance perfusion of targeted tumor tissue. This dual functionality permits spatially localized heating of brain and tumor tissue to decrease IFP and increase tissue permeability to the drug. Each microneedle surface can be bevel polished and/or acid-etched to effectively distribute the photothermal dose to heat tissue to only 42° C. and prevent thermal injury. Due to intrinsically higher absorption of optical energy by the white matter and tumor tissue, the temperature increase (and permeability increase) is desirably greater in these targets. Clinical and laboratory evidence has shown that hyperthermia (elevated temperature) enhances the cytotoxic effects of several chemotherapeutic drugs(thermo-chemotherapy) for intraperitoneal tumors. This observed enhancement may be due in part to increased tumor cell membrane permeability and greater drug metabolism by the cells. Photothermal damage is minimized by maintaining temperature below ~42° C., previously shown to be effective for intraperitoneal hyperthermic chemotherapy (C. R. Rossi et al., Cancer, 2002), and mild hyperthermia of gliomas (T. Uzuka, et al., Neurol Med Chir (Tokyo), 2006). Hyperthermia may also provide a means to increase regional circulatory vascular dilation and perfusion, which has implications for more effective perfusion of tumor tissue with CED. However, no studies have investigated the effect of CED perfusion of gliomas with laser-induced local hyperthermia. An experiment was conducted to evaluate the temperature and time effect on QUAD-DM1. QUAD-DM1 of varying molarity (10⁻¹² to 10⁻⁸) was brought to 37, 45, or 60° C. and held for up to 240 min. After returning to 25° C., the QUAD-DM1 was exposed to U-251 glioblastoma cells. A loss of only a log of QUAD-DM1 activity was observed only at 60° C. for durations >=120 minutes. This extreme combination of time/temperature however not used with the disclosed CETCS system, therefore there is no concern of thermal interaction with the drug. Additionally, an experiment was conducted to evaluate potential photosensitive cytotoxic degradation of QUAD-DM1. 1064 nm laser exposure of 200 mW for 120 minutes (=24 Joules) had no effect on the cytotoxic potency of the QUAD-DM1.

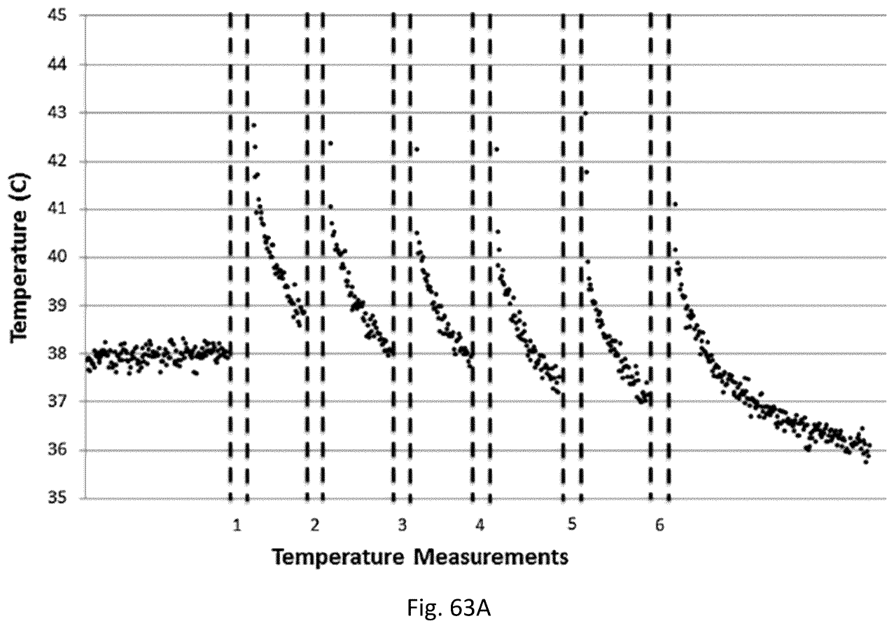

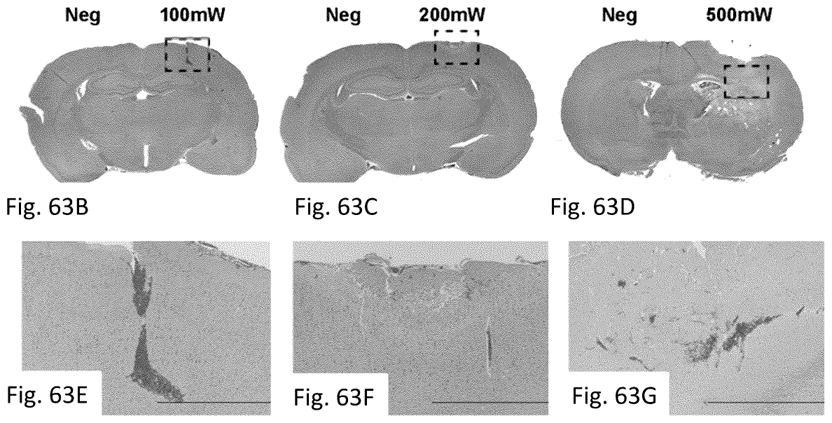

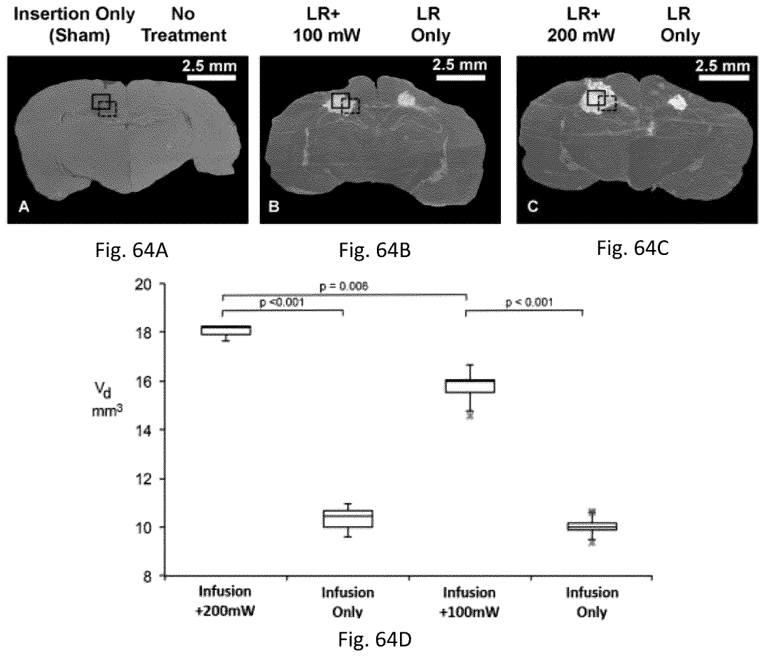

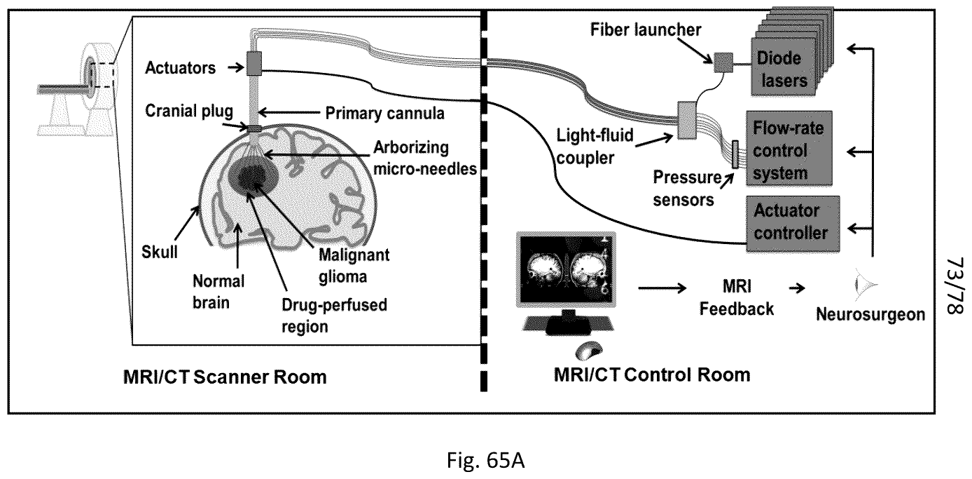

CED thermo-therapy achieves greater infusion dispersal in tissue. Studies have previously been conducted to determine the threshold laser power to cause thermal injury while enhancing dispersal volume (Va). Laser power ranging from 0 to 750 mW was delivered to brains of live rats using a single fiberoptic microneedle. Data collected by turning off the laser for 15-sec intervals every 10 mins produced repeatable data for the 100 and 200 mW groups (FIG. 63A). The average steady-state temperature was 38.7±1.6 and 42.0=1.0° C. for laser irradiation at 100 and 200 mW, respectively. Histological evaluation of the tissue surrounding the irradiation site did not indicate thermal damage for 0 mW and 100 mW irradiations (FIG. 63B, FIG. 63E). Thermal damage was detected at 200 mW, although it was limited to a small penumbra of cerebral cortical microcavitation and necrosis that immediately surrounded the region of microneedle insertion (FIG. 63C, FIG. 63F). In rats treated at laser powers greater than 200 mW, there were extensive regions of thermal damage (FIG. 63D, FIG. 63G), suggesting that laser power delivered to tissue should remain at or below the 200 mW threshold. In a corresponding study, it was shown that laser energy produced greater dispersion of co-delivered liposomal rhodamine (LR) infused using a single co-delivery microneedle in brains of anesthetized rats. Laser powers (1,064 nm wavelength) at 0 (control), 100, and 200 mW were investigated. The greater dispersion of LR (yellow-gold) with co-delivered laser power (100 mW shown in FIG. 64B and 200 mW shown in FIG. 64C) can be seen in fluorescent brain slices compared to infusion of the dye without laser power (fluid-only control). V_{d} for 100 mW of laser power were 60% greater compared to its fluid-only control (15.8±0.6 mm³ vs 10.0±0.4 mm³, respectively) and 80% greater for 200 mW of laser power compared to its fluid-only control (18.0±0.3 mm³ vs 10.3±0.7mm³) (p<0.001) (FIG. 64D).

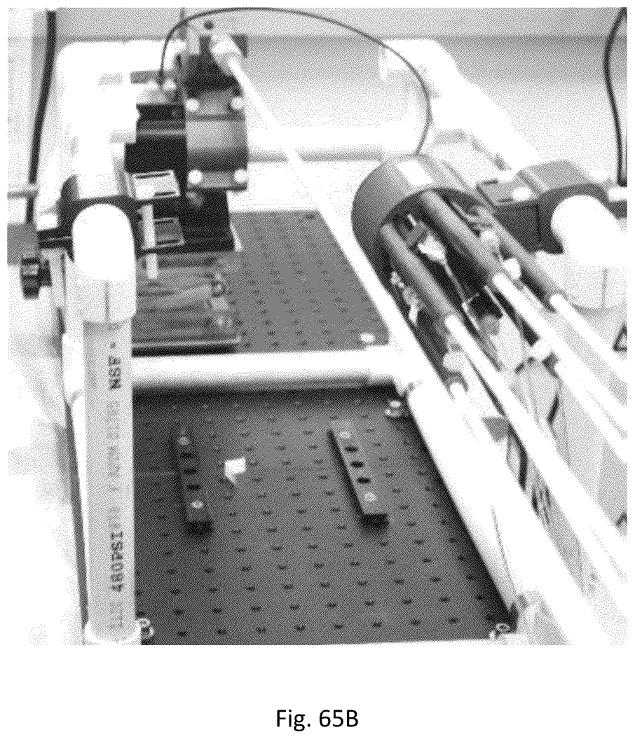

Described herein is a remote-control CETCS (operable from an MRI or CT control room) that adjusts the treatment parameters (e.g., cannula and microneedle positions, drug flow rate/pressure, and light dose) without removing the patient from the scanner as shown in FIG. 65A. The actuation mechanisms (FIG. 65B) were developed to control 8 degrees of freedom: cannula deployment, cannula rotation, and deployment of 6 microneedles. Position input to the actuation mechanisms is controlled using electromechanical stepper motors. A transmission system of rotating fiberglass rods keeps the stepper motors a safe distance away from the MRI scanner.

A digital camera was used to obtain position data during in-plane movements of the microneedles deployed into agarose tissue phantoms. This data was used to calculate position repeatability as well as examine backlash hysteresis (positioning error due to direction change) according to the ISO 9283 standard. Microneedle deployment repeatability ranged from 0.05 to 0.24 mm. Backlash hysteresis ranged from 0.7 mm for the center microneedle to 2.0 mm for needles deploying from the side of the cannula, but backlash hysteresis was constant for individual needles and the control system algorithm was modified to compensate for backlash. Therefore, CETCS was demonstrated to achieve position repeatability and backlash within the design specification of less than 1.0 mm.

A control algorithm was developed to allow a syringe pump to be used as a constant pressure source, and the ability of constant pressure infusions coupled with controlled catheter movement was shown to increase V_{d} in agarose gel brain tissue phantoms. Constant flow rate and constant pressure infusions were conducted with a stationary catheter, a catheter retracting at a rate of 0.25 mm/min, and a catheter retracting at a rate of 0.5 mm/min. The 0.25 mm/min and 0.5 mm/min retracting constant pressure catheters resulted in significantly larger V_{d} compared to any other group, with a 105% increase and a 155% increase compared to the stationary constant flow rate catheter, respectively (see Table 2 below). Additionally, these same constant pressure retracting infusions resulted in 42% and 45% increase in V_{d} compared to their constant flow rate counterparts. Table 2 shows the results from the five infusions for each group. V_{d} for each group was significantly different from other groups (p<0.001) with the exception of the two stationary catheters which were statistically the same (p=0.93). The infusions were each 100 min., and the data in Table 2 is represented as mean±standard deviation.

TABLE 2

| | | | | Pressure | Flow Rate |
|---|---|---|---|---|---|
| Group | V_{d} (mL) | Vᵢ (mL) | V_{d}:Vᵢ | (Pa) | (μL/min) |
| Stationary Const. | 1.55 ± 0.03 | 0.1 | 15.5 ± 0.32 | 977 ± 160 | 1 |
| Flow | | | | | |
| 0.25 mm/min Const. | 2.23 ± 0.04 | 0.1 | 22.3 ± 0.35 | 306 ± 142 | 1 |
| Flow | | | | | |
| 0.5 mm/min Const. | 2.74 ± 0.05 | 0.1 | 27.4 ± 0.53 | 239 ± 154 | 1 |
| Flow | | | | | |
| Stationary Const. | 1.63 ± 0.07 | 0.11 ± 0.01 | 14.71 ± 0.63 | 967 ± 93 | 1.11 ± 0.15 |
| Pressure | | | | | |
| 0.25 mm/min Const. | 3.17 ± 0.25 | 0.24 ± 0.01 | 13.19 ± 1.21 | 945 ± 87 | 2.41 ± 0.42 |
| Pressure | | | | | |
| 0.5 mm/min Const. | 3.96 ± 0.19 | 0.29 ± 0.02 | 13.69 ± 0.92 | 942 ± 95 | 2.90 ± 0.54 |
| Pressure | | | | | |

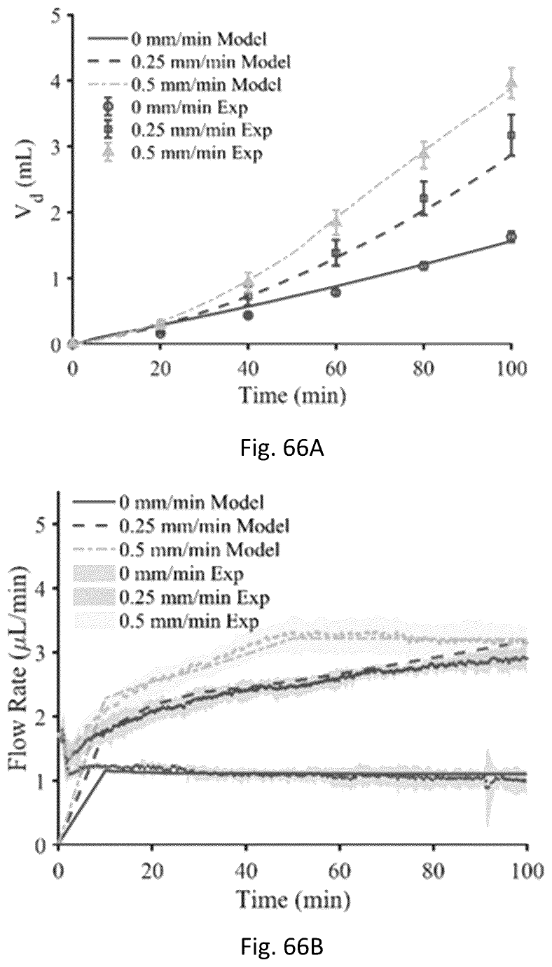

Also disclosed herein is a computational model to predict V_{d} achieved by various retraction rates with both constant pressure and constant flow rate infusions. Briefly, the biphasic-solute material model allows for the transport of a solute in porous media via both diffusion and convection and has consistently been used in the literature to model CED. The governing equations describing the balance of linear momentum for the biphasic-solute material were utilized. In order to conduct a parametric study on needle retraction speed, a material model was used (E. Y. Elenes, et al., Journal of Engineering and Science in Medical Diagnostics and Therapy, August 2019). The Holmes-Mow relation for the strain-dependent hydraulic permeability tensor was reduced to form the relation proposed by Lai and Mow (W. M. Lai et al., Biorheology, 1980) The resulting model shows an increase in V_{d} is achieved with any retraction rate, and the amount of V_{d} between successive retraction rates drops off at rates above 0.3 to 0.35 mm/min (FIG. 66A and FIG. 66B) (J. Mehta, et al., Numerical Methods in Biomedical Engineering, June 2022). Finally, it was found that infusions with retraction result in a more even distribution in concentration level compared to the stationary catheter, suggesting a potential ability for retracting catheters to have a therapeutic advantage. These experiments were performed using homogeneous agarose phantoms at room temperature; therefore, more variable results should be expected due to heterogeneous tissue properties in vivo.

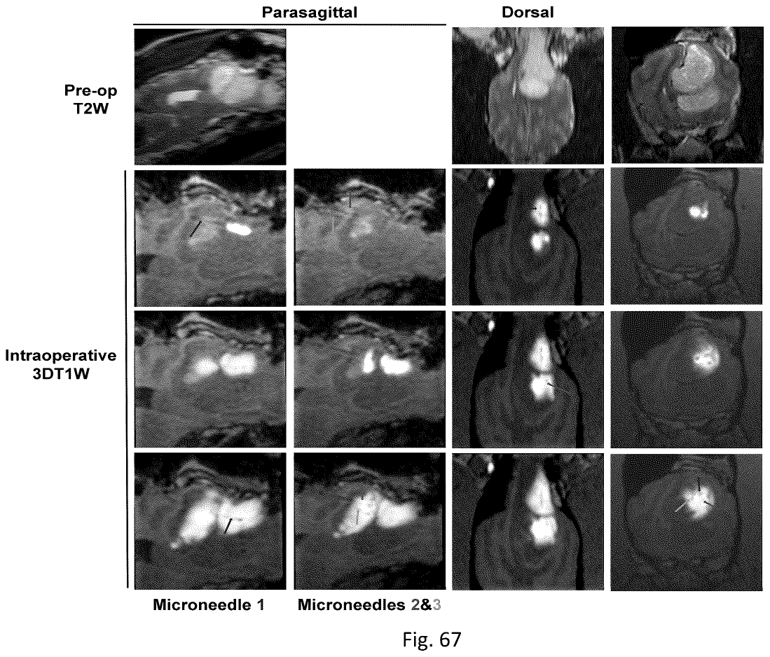

CETCS hardware and control software has been successfully integrated into the CT and MRI environments. Arborization of CETCS microneedles was used to deliver QUAD conjugates (QUAD-CTX) to three canine patients with gliomas. It has been shown that it is possible to infuse a non-enhancing tumor with CETCS and reach infiltrating tumor cells with drugs (FIG. 67). >55% tumor coverage was achieved in all 3 dogs treated with CETCS, and >70% tumor coverage in 2/3 dogs.

Integration with MR Imaging Feedback and Repositioning Software

To address non-symmetric drug distribution and lower-than-desirable drug coverage of tumor tissue by CED, in some embodiments, CETCS synergistically integrated with MR imaging feedback and repositioning software can cover missed tumor volume following the initial infusion, and thereby achieve >80% coverage of target volume (defined as MR T2 hyperintense region) with QUAD-DM1+Gd-alb surrogate MR imaging tracer in canines with spontaneous MGs.

The anatomical heterogeneity of the brain and tumor tissue; differences in permeability between white and gray matter; and issues arising from low-pressure “sinks”, such as cerebrospinal fluid spaces, are all challenges for CED perfusion that result in non-symmetric drug distribution and lower-than-desirable drug coverage of tumor tissue. To address these challenges and take advantage of the disclosed remotely-operable CETCS system, in some embodiments, the actual 3D spatial distribution of Gd-alb surrogate tracer are analyzed post-CETCS-treatment. Based on the “missed” 3D regions of target tumor not covered with of Gd-alb, the CETCS may in some embodiments be repositioned for a second-treatment infusion to increase target volume coverage. In recent years, the field of image-guided surgery utilizing MR and CT feedback has grown significantly because it provides the surgeon visual feedback at the surgery site. In some embodiments of the disclosed system, some or all of CETCS, MR, and computational modeling/software are integrated for a first-of-its-kind image-guided CED therapy.

In some embodiments, pre-treatment images will include MR T1 contrast to provide detail of sensitive anatomic features including major blood vessels and ventricles, as well as MR T2 contrast to map the hyperintense region which will serve as the target tumor volume for the therapy. Software packages such as 3-D Slicer may be used for image segmentation and 3D reconstruction of target tumor and sensitive anatomic features. A neurosurgeon may then use these 3D models for planning the initial CETCS treatment; specifically deciding on the CETCS main cannula point of insertion through the skull and trajectory within the brain to cover tumor while avoiding sensitive anatomic structures. Surgical placement of the cranial implantable PGP and CETCS initial treatment may be performed as described below.

Immediately following the CETCS initial treatment, post-treatment MR T1 scans may be performed to provide contrast for QUAD-DM1+Gd-alb surrogate tracer distribution as well as CETCS cannula and microneedle locations (as shown in FIG. 67). Regions of peritumoral edema may be excluded from the tumor and drug volume by composite modeling of tumor geometry using available MR sequences (T1 pre- and post-treatment, T2, and T2 FLAIR). Using software for image segmentation, 3D reconstruction, and co-registration with the pre-treatment images, a 3D model is then created of the CETCS catheter and of Gd-alb distribution, and the two models may be overlaid on the pre-treatment 3D models of target tumor and sensitive anatomic features. Using the intersection of the target tumor and Gd-alb models, a 3D model of target tumor covered by Gd-alb(therapeutic region) may then be generated. Similarly, a 3D model of target tumor missed by Gd-alb (non-therapeutic region) may be created. These models may then be used to calculate V_{d} and % V_{target}.

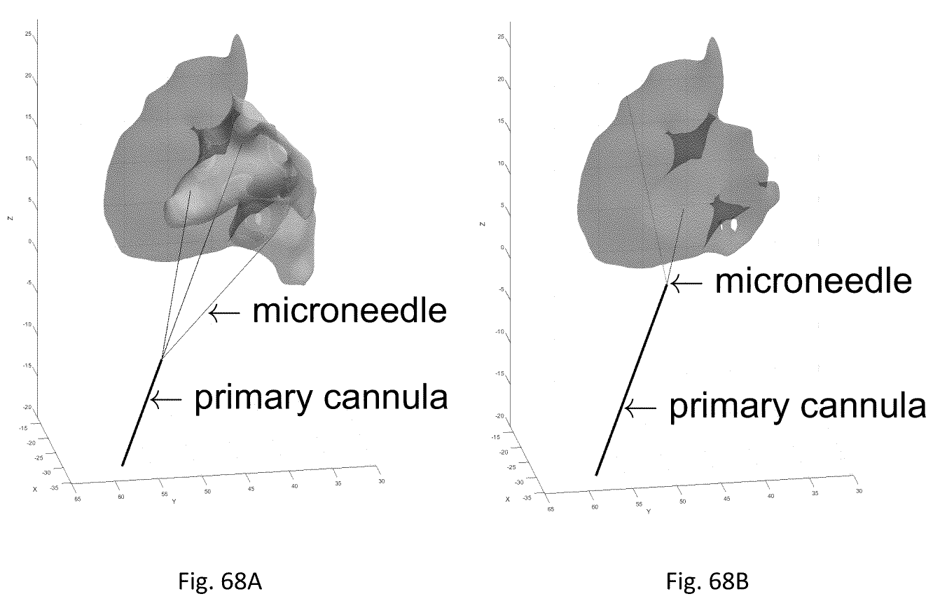

The dimensionally accurate co-registered 3D geometric models are then imported into MATLAB software, where matrix manipulation functions are used to create a kinematic (geometrically adjustable) mathematical model for the CETCS cannula and microneedles (FIG. 68A and FIG. 68B). The kinematic model may be constructed having 8 degrees of freedom (DOF): cannula deployment distance, cannula rotation, and independent deployment distance for each of the 6 microneedles. The constraints on the model may include some or all of upper limits on cannula and microneedle deployment distance (10 cm, and 50 mm, respectively, due to catheter design); as well as cannula rotation (72°, due to radial angular spacing between the 5 side microneedles). The trajectory of the main cannula may be fixed due to the cranial implantable PGP. The azimuthal angle) (30° of the side microneedles relative to the cannula and center microneedle may also be fixed by design. The initial conditions of the kinematic model may be prescribed based on a best-fit to the 3D geometric model of CETCS cannula and microneedles generated from MR images. FIG. 68A shows the kinematic model overlaying the 3D geometric model of target tumor and Gd-alb distribution based on a canine treatment with CETCS.

Next, the disclosed CETCS repositioning algorithm will predict the best combination of all 8 DOF of the kinematic model to achieve maximum coverage of missed tumor while avoiding sensitive anatomic features. 10 linearly-spaced values for each of the 8 DOF within their allowed range are examined. In some embodiments, all 108 possible combinations of values of DOF are examined in under 5 minutes. For each combination, the algorithm calculates the amount of missed tumor volume within a 1 cm distance of all microneedle tracks deployed (i.e.; from the initial maximum deployment position to the completely retracted state at the tip of the cannula). The algorithm eliminates all DOF combinations where one or more microneedle tracks pass within 1 cm of a sensitive anatomic feature. The final outputs of the repositioning software are the recommended values for repositioned CETCS main cannula deployment length and rotation, as well as the deployment length of each independent microneedle for a second treatment. Data demonstrating repositioned CETCS is shown in FIG. 68B. Note the primary cannula is inserted toward the tumor, rotated 10°, and microneedles #3and #5are redeployed independent distances. The 3D model of sensitive anatomic features is suppressed in FIG. 68A and FIG. 68B for improved visibility.

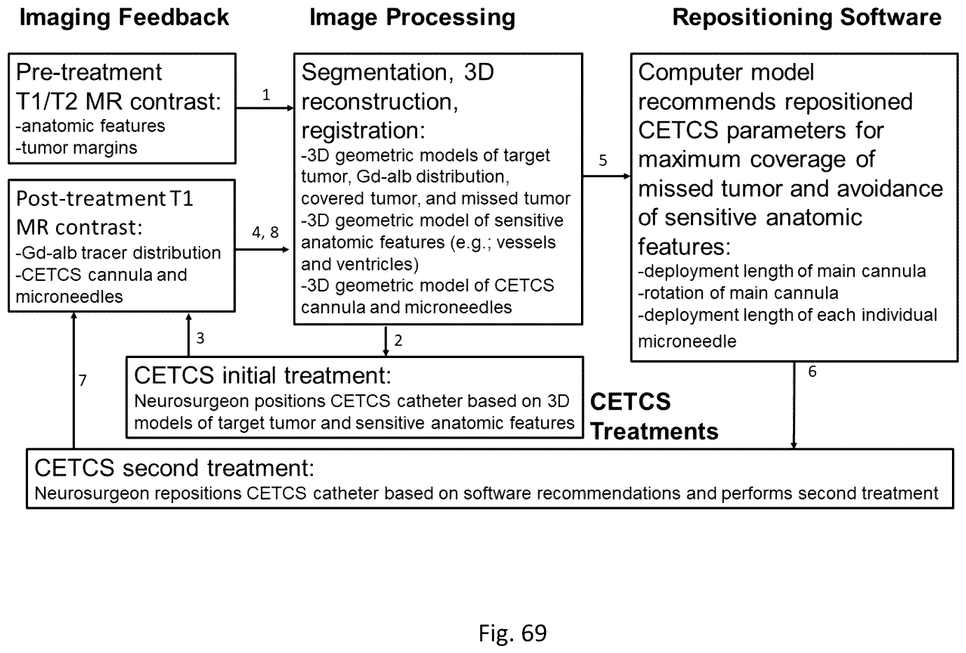

A process flow diagram of an exemplary adaptive treatment strategy using CETCS is outlined in FIG. 69. Based on the recommendations of the repositioning software, a neurosurgeon may implement the repositioned states of CETCS cannula and microneedles into existing control software. The existing hardware (see FIG. 65A) first retracts all microneedles and then remotely adjust cannula deployment, cannula rotation, and independent microneedle deployment without removing the patient from the MR scanner, nor extracting the cannula from its initial insertion point. CETCS treatment and post-treatment MR imaging are performed again, yielding a final combined first-and-second-treatment V_{d} and % V_{target} which is compared to the results of the first-treatment on a per-patient basis.

In one embodiment, a microneedle repositioning system may comprise the steps of voxelizing a three-dimensional model of the brain, including ventricles and identified regions (i.e., target tumor, therapeutic and non-therapeutic regions) with a resolution of 1 mm×1 mm×1 mm to simulate a planning environment. These voxels may then be used for obstacle detection and characterizing volumetric coverage. Next, all feasible microneedle repositions that target the non-therapeutic region will be identified using, for example, a “brute-force” approach. Repositioning configurations will be evaluated using a cost function of

$$c ( E ) = {a}_{1} {f}_{V} + {a}_{2} {f}_{D}$$

where f_{V} and f_{D} are cost terms penalizing volume fraction of coverage of the non-therapeutic region, and each microneedle's distance from ventricles (obstacles) respectively, and a₁ and a₂ (1/mm) are the weights regulating the relative importance of each cost term;

$${f}_{V} = \frac{{V}_{perfused}}{{V}_{tumor}}$$

$${f}_{D} = \frac{1}{{N}_{0}} {\sum }_{i}^{{N}_{0}} \frac{{ϵ}_{d} - {r}_{c}}{{d}_{i} - {r}_{c}}$$

$${d}_{i} = \{ \begin{matrix}{d}_{i} ; & {r}_{c} < {d}_{i} \leq {ϵ}_{d} \\ {ϵ}_{d} ; & {ϵ}_{d} < {d}_{i}\end{matrix}$$

where N₀ is the number of obstacles, ∈_{d} is the Maximum distance above which the cost function is not affected, r_{c} is the Minimum admissible distance from obstacle (1 mm in canine brain), dᵢ is the Distance of the i-th obstacle to the microneedle, V_{perfused} is the Volume of the target tumor perfused, and Vₜᵤₘₒᵣ is the Volume of the target tumor.

In some embodiments, a rapidly-exploring random tree (RRT*) algorithm then proposes best feasible paths based on c(E).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. The numbered arrows in FIG. 69 denote order of operations.

### REFERENCES

The following publications are incorporated by reference in their entirety.
R. Siegel, D. Naishadham, and A. Jemal, “Cancer statistics, 2013,” CA: A Cancer Journal for Clinicians, vol. 63, no. 1, pp. 11-30, 2013, doi: 10.3322/caac.21166.
“Cancer Facts & FIGS. 2019 American Cancer Society.” 2019. [Online]. Available: https://www.cancer.org/research/cancer-facts-statistics/all-cancer-facts-figures/cancer-facts-figures-2019.html
Q. T. Ostrom et al., “CBTRUS Statistical Report: Primary Brain and Central Nervous System Tumors Diagnosed in the United States in 2008-2012,” Neuro-Oncology, vol. 17, no. suppl_4, pp. iv1-iv62, October 2015, doi: 10.1093/NEUONC/NOV189.
F. H. Hochberg and A. Pruitt, “Assumptions in the radiotherapy of glioblastoma,” Neurology, vol. 30, no. 9, p. 907, September 1980, doi: 10.1212/WNL.30.9.907.
M. S. Berger, “Malignant astrocytomas: surgical aspects,” Seminars in Oncology, vol. 21, no. 2, pp. 172-185, April 1994.
E. G. Shaw, “Central nervous system overview,” Clinical radiation oncology, pp. 314-335, 2000.
G. J. Lesser and S. A. Grossman, “The chemotherapy of adult primary brain tumors,” Cancer Treatment Reviews, vol. 19, no. 3, pp. 261-281, July 1993, doi: 10.1016/0305-7372 (93) 90038-S.
R. Nishikawa, “Standard Therapy for Glioblastoma-A Review of Where We Are,” Neurol Med Chir (Tokyo), vol. 50, no. 9, pp. 713-719, 2010, doi: 10.2176/nmc.50.713.
R. Stupp et al., “Radiotherapy plus Concomitant and Adjuvant Temozolomide for Glioblastoma,” New England Journal of Medicine, vol. 352, no. 10, pp. 987-996, March 2005, doi: 10.1056/NEJMoa043330.
P. F. Morrison, D. W. Laske, H. Bobo, E. H. Oldfield, and R. L. Dedrick, “High-flow microinfusion: tissue penetration and pharmacodynamics,” American Journal of Physiology-Regulatory, Integrative and Comparative Physiology, vol. 266, no. 1, pp. R292-R305, January 1994, doi: 10.1152/ajpregu. 1994.266.1.R292.
N. U. Barua, S. P. Lowis, M. Woolley, S. O'Sullivan, R. Harrison, and S. S. Gill, “Robot-guided convection-enhanced delivery of carboplatin for advanced brainstem glioma,” Acta Neurochirurgica, vol. 155, no. 8, pp. 1459-1465 Aug. 2013, doi: 10.1007/s00701-013-1700-6.
S. Kunwar et al., “Phase III randomized trial of CED of IL13-PE38QQR vs Gliadel wafers for recurrent glioblastomat,” Neuro-Oncology, vol. 12, no. 8, pp. 871-881, August 2010, doi: 10.1093/neuonc/nop054.
W. A. Vandergrift, S. J. Patel, J. S. Nicholas, and A. K. Varma, “Convection-enhanced delivery of immunotoxins and radioisotopes for treatment of malignant gliomas,” Neurosurgical Focus, vol. 20, no. 4, p. E13, April 2006, doi: 10.3171/FOC.2006.20.4.8.
R. H. Bobo, D. W. Laske, A. Akbasak, P. F. Morrison, R. L. Dedrick, and E. H. Oldfield, “Convection-enhanced delivery of macromolecules in the brain.,” Proceedings of the National Academy of Sciences, vol. 91, no. 6, pp. 2076-280 Mar. 1994, doi: 10.1073/pnas.91.6.2076.
R. Raghavan, M. L. Brady, M. I. Rodríguez-Ponce, A. Hartlep, C. Pedain, and J. H. Sampson, “Convection-enhanced delivery of therapeutics for brain disease, and its optimization,” Neurosurgical Focus, vol. 20, no. 4, p. E12, April 2006, doi: 10.3171/FOC.2006.20.4.7.
W. Debinski and S. B. Tatter, “Convection-enhanced delivery for the treatment of brain tumors,” Expert Review of Neurotherapeutics, vol. 9, no. 10, pp. 1519-1527 Oct. 2009, doi: 10.1586/ern.09.99.
W. Yang et al., “Convection enhanced delivery of carboplatin in combination with radiotherapy for the treatment of brain tumors,” Journal of Neuro-Oncology, vol. 101, no. 3, pp. 379-390, February 2011, doi: 10.1007/s11060-010-0272-z.
M. A. Vogelbaum and M. K. Aghi, “Convection-enhanced delivery for the treatment of glioblastoma,” Neuro-Oncology, vol. 17, no. suppl_2, pp. ii3-ii8, March 2015, doi: 10.1093/neuonc/nou354.
D. Ding et al., “Convection-enhanced delivery of free gadolinium with the recombinant immunotoxin MR1-1,” Journal of Neuro-Oncology, vol. 98, no. 1, pp. 1-7, May 2010, doi: 10.1007/s11060-009-0046-7.
Y. Mardor et al., “Convection-Enhanced Drug Delivery of Interleukin-4 Pseudomonas Exotoxin (PRX321): Increased Distribution and Magnetic Resonance Monitoring,” Journal of Pharmacology and Experimental Therapeutics, vol. 330, no. 2, p. 520, August 2009, doi: 10.1124/jpet. 109.154401.
Y. Mardor et al., “Convection-Enhanced Drug Delivery: Increased Efficacy and Magnetic Resonance Image Monitoring,” Cancer Research, vol. 65, no. 15, pp. 6858-6863 Aug. 2005, doi: 10.1158/0008-5472.CAN-05-0161.
R. A. Patchell et al., “A Phase I Trial of Continuously Infused Intratumoral Bleomycin for the Treatment of Recurrent Glioblastoma Multiforme,” Journal of Neuro-Oncology, vol. 60, no. 1, pp. 37-42, October 2002, doi: 10.1023/A: 1020291229317.
D. A. Bosch, Th. Hindmarsch, St. Larsson, and E.-O. Backlund, “Intraneoplastic Administration of Bleomycin in Intracerebral Gliomas: A Pilot Study,” in Advances in Stereotactic and Functional Neurosurgery 4, 1980, pp. 441-444.
M. S. Fiandaca, J. R. Forsayeth, P. J. Dickinson, and K. S. Bankiewicz, “Image-guided convection-enhanced delivery platform in the treatment of neurological diseases,” Neurotherapeutics, vol. 5, no. 1, pp. 123-127, January 2008, doi: 10.1016/j.nurt.2007.10.064.
M. T. Krauze et al., “Reflux-free cannula for convection-enhanced high-speed delivery of therapeutic agents: Technical note,” Journal of Neurosurgery, vol. 103, no. 5, pp. 923-929, November 2005, doi: 10.3171/JNS.2005.103.5.0923.
J. H. Sampson et al., “Poor drug distribution as a possible explanation for the results of the PRECISE trial: Clinical article,” Journal of Neurosurgery, vol. 113, no. 2, pp. 301-309, August 2010, doi: 10.3171/2009.11.JNS091052.
M. Mut, J. H. Sherman, M. E. Shaffrey, and D. Schiff, “Cintredekin besudotox in treatment of malignant glioma,” Expert Opinion on Biological Therapy, vol. 8, no. 6, pp. 805-812, June 2008, doi: 10.1517/14712598.8.6.805.
E. Y. Elenes et al., “Convection-Enhanced Arborizing Catheter System Improves Local/Regional Delivery of Infusates Versus a Single-Port Catheter in Ex Vivo Porcine Brain Tissue,” Journal of Engineering and Science in Medical Diagnostics and Therapy, vol. 4, no. 1, December 2020, doi: 10.1115/1.4048935.
J. N. Mehta, G. R. McRoberts, and C. G. Rylander, “Controlled Catheter Movement Affects Dye Dispersal Volume in Agarose Gel Brain Phantoms,” Pharmaceutics, vol. 12, no. 8, p. 753, August 2020, doi: 10.3390/pharmaceutics12080753.
“CBTRUS Statistical Report: Primary Brain and Central Nervous System Tumors Diagnosed in the United States in 2008-2012 Neuro-Oncology Oxford Academic.” [Online]. Available: https://academic.oup.com/neuro-oncology/article/17/suppl_4/iv1/4107103?login=false
K. J. Denton, D. W. Cotton, R. A. Nakielny, and J. R. Goepel, “Secondary tumour deposits in needle biopsy tracks: an underestimated risk?,” Journal of Clinical Pathology, vol. 43, no. 1, p. 83, January 1990, doi: 10.1136/jcp.43.1.83-a.
H. S. Schwartz and D. M. Spengler, “Needle tract recurrences after closed biopsy for sarcoma: Three cases and review of the literature,” Annals of Surgical Oncology, vol. 4, no. 3, pp. 228-236, 1997, doi: 10.1007/BF02306615.
C. Slywotzky and M. Maya, “Needle tract seeding of transitional cell carcinoma following fine-needle aspiration of a renal mass,” Abdominal Imaging, vol. 19, no. 2, pp. 174-176, March 1994, doi: 10.1007/BF00203498.
L. C. Vazquez et al., “Polymer-coated cannulas for the reduction of backflow during intraparenchymal infusions,” Journal of Materials Science: Materials in Medicine, vol. 23, no. 8, pp. 2037-246 Aug. 2012, doi: 10.1007/s10856-012-4652-0.
C. Rylander and et.al., “Fiber array for optical imaging and therapeutics,” U.S. Pat. No. 8,798,722B2, 2014
VIRGINIA TECH INTELLECTUAL PROPERTIES INC, “US Patent for Fiber array for optical imaging and therapeutics Patent (Patent #10,220,124),” 2019 [Online]. Available: https://patents.justia.com/patent/10220124
C. Rylander and et.al., “Convection-Enhanced Thermo-chemotherapy Catheter System (CETCS),” Provisional Patent Application 63/309,227 filed Feb. 11, 2022.
E. Elenes and C. Rylander, Maximizing Local Access to Therapeutic Deliveries in Glioblastoma. Part II: Arborizing Catheter for Convection-Enhanced Delivery in Tissue Phantoms. Glioblastoma, Brisbane, Australia: Codon Publications, 2017.
E. Y. Elenes et al., “Convection-Enhanced Arborizing Catheter System Improves Local/Regional Delivery of Infusates Versus a Single-Port Catheter in Ex Vivo Porcine Brain Tissue,” Journal of Engineering and Science in Medical Diagnostics and Therapy, vol. 4, no. 1, December 2020, doi: 10.1115/1.4048935.
G. P. Mazzara, R. P. Velthuizen, J. L. Pearlman, H. M. Greenberg, and H. Wagner, “Brain tumor target volume determination for radiation treatment planning through automated MRI segmentation,” International Journal of Radiation Oncology*Biology*Physics, vol. 59, no. 1, pp. 300-312, May 2004, doi: 10.1016/J.IJROBP.2004.01.026.
M. F. Dempsey, B. R. Condon, and D. M. Hadley, “Measurement of Tumor ‘Size’ in Recurrent Malignant Glioma: 1D, 2D, or 3D?,” American Journal of Neuroradiology, vol. 26, no. 4, p. 770, April 2005, [Online]. Available: http://www.ajnr.org/content/26/4/770.abstract
R. L. Hood, J. H. Rossmeisl, R. T. Andriani, A. R. Wilkinson, J. L. Robertson, and C. G. Rylander, “Intracranial hyperthermia through local photothermal heating with a fiberoptic microneedle device,” Lasers in Surgery and Medicine, vol. 45, no. 3, pp. 167-174, March 2013, doi: 10.1002/LSM.22117.
R. L. Hood, R. T. Andriani, S. Emch, J. L. Robertson, C. G. Rylander, and J. H. Rossmeisl, “Fiberoptic microneedle device facilitates volumetric infusate dispersion during convection-enhanced delivery in the brain,” Lasers in Surgery and Medicine, vol. 45, no. 7, pp. 418-426, September 2013, doi: 10.1002/LSM.22156.
R. L. Hood, R. T. Andriani, T. E. Ecker, J. L. Robertson, and C. G. Rylander, “Characterizing Thermal Augmentation of Convection-Enhanced Drug Delivery with the Fiberoptic Microneedle Device,” Engineering, vol. 1, no. 3, pp. 344-350, 2015, doi: https://doi.org/10.15302/J-ENG-2015077.
C. Nicholson and L. Tao, “Diffusion Properties of Brain Tissue Measured with Electrode Methods and Prospects for Optical Analysis,” in Optical Imaging of Brain Function and Metabolism, U. Dirnagl, A. Villringer, and K. M. Einhäupl, Eds. Boston, MA: Springer US, 1993, pp. 213-223. doi: 10.1007/978-1-4899-2468-1 19.
S. C. Gebhart, W. C. Lin, and A. Mahadevan-Jansen, “In vitrodetermination of normal and neoplastic human brain tissue optical properties using inverse adding-doubling,” Physics in Medicine and Biology, vol. 51, no. 8, pp. 2011-227 Mar. 2006, doi: 10.1088/0031-9155/51/8/004.
H. R. Eggert and V. Blazek, “Optical properties of human brain tissue, meninges, and brain tumors in the spectral range of 200 to 900 nm,” Neurosurgery, vol. 21, no. 4, pp. 459-464, 1987.
M. R. DeWitt, A. M. Pekkanen, J. Robertson, C. G. Rylander, and M. Nichole Rylander, “Influence of Hyperthermia on Efficacy and Uptake of Carbon Nanohorn-Cisplatin Conjugates,” Journal of Biomechanical Engineering, vol. 136, no. 2, February 2014, doi: 10.1115/1.4026318.
C. R. Rossi et al., “Hyperthermic intraoperative intraperitoneal chemotherapy with cisplatin and doxorubicin in patients who undergo cytoreductive surgery for peritoneal carcinomatosis and sarcomatosis,” Cancer, vol. 94, no. 2, pp. 492-499, 2002, doi: 10.1002/cncr.10176.
T. UZUKA, H. TAKAHASHI, and R. TANAKA, “Interstitial Hyperthermia With Intra-arterial Injection of Adriamycin for Malignant Glioma,” Neurol Med Chir (Tokyo), vol. 46, no. 1, pp. 19-23, 2006, doi: 10.2176/nmc.46.19.
“Modeling of Neutral Solute Transport in a Dynamically Loaded Porous Permeable Gel: Implications for Articular Cartilage Biosynthesis and Tissue Engineering J. Biomech Eng. ASME Digital Collection.” [Online]. Available: https://asmedigitalcollection.asme.org/biomechanical/article/125/5/602/449172/Modeling-of-Neutral-Solute-Transport-in-a
E. Y. Elenes, M. K. Rausch, and C. G. Rylander, “Parametric Study of the Design Variables of an Arborizing Catheter on Dispersal Volume Using a Biphasic Computational Model,” Journal of Engineering and Science in Medical Diagnostics and Therapy, vol. 2, no. 3, August 2019, doi: 10.1115/1.4042874.
W. M. Lai and V. C. Mow, “Drag-induced compression of articular cartilage during a permeation experiment,” Biorheology, vol. 17, no. 1-2, pp. 111-123, January 1980, doi: 10.3233/BIR-1980-171-213.
J. Mehta, M. Rausch, and C. Rylander, “Convection-Enhanced Delivery with Controlled Catheter Movement: A Parametric Finite Element Analysis—In review,” Numerical Methods in Biomedical Engineering.
C. for D. and R. Health, “Medical Devices; Current Good Manufacturing Practice (CGMP) Final Rule; Quality System Regulation,” FDA, November 2018, [Online]. Available: https://www.fda.gov/medical-devices/quality-system-qs-regulationmedical-device-good-manufacturing-practices/medical-devices-current-good-manufacturing-practice-cgmp-final-rule-quality-system-regulation
Z. J. Chen et al., “A realistic brain tissue phantom for intraparenchymal infusion studies,” Journal of Neurosurgery, vol. 101, no. 2, pp. 314-322, August 2004, doi: 10.3171/JNS.2004.101.2.0314.
C. Pena, “K161731 Substantial Equivalence Determination Letter,” 2017.
R. R. Lonser et al., “Successful and safe perfusion of the primate brainstem: in vivo magnetic resonance imaging of macromolecular distribution during infusion,” Journal of Neurosurgery, vol. 97, no. 4, pp. 905-913, October 2002, doi: 10.3171/JNS.2002.97.4.0905.
P. J. Dickinson et al., “Canine model of convection-enhanced delivery of liposomes containing CPT-11 monitored with real-time magnetic resonance imaging: Laboratory investigation,” Journal of Neurosurgery, vol. 108, no. 5, pp. 989-998, May 2008, doi: 10.3171/JNS/2008/1Aug. 5, 0989.
D. Ding, C. W. Kanaly, T. J. Cummings, J. E. Herndon, R. Raghavan, and J. H. Sampson, “Long-term safety of combined intracerebral delivery of free gadolinium and targeted chemotherapeutic agent PRX321,” Neurological Research, vol. 32, no. 8, pp. 810-815, October 2010, doi: 10.1179/174367509X12581069052090.
J. H. Rossmeisl et al., “Phase I trial of convection-enhanced delivery of IL13RA2 and EPHA2 receptor targeted cytotoxins in dogs with spontaneous intracranial gliomas,” Neuro-Oncology, vol. 23, no. 3, pp. 422-434, March 2021, doi: 10.1093/neuonc/noaa196.
A. I. Mehta et al., “Imaging of Convection Enhanced Delivery of Toxins in Humans,” Toxins (Basel), vol. 3, no. 3, pp. 201-206, March 2011, doi: 10.3390/toxins3030201.
J. Liu, J. K. Udupa, D. Odhner, D. Hackney, and G. Moonis, “A system for brain tumor volume estimation via MR imaging and fuzzy connectedness,” Computerized Medical Imaging and Graphics, vol. 29, no. 1, pp. 21-34, January 2005, doi: 10.1016/j.compmedimag.2004.07.008.
M. Vaidyanathan et al., “Monitoring brain tumor response to therapy using MRI segmentation,” Magnetic Resonance Imaging, vol. 15, no. 3, pp. 323-334, January 1997, doi: 10.1016/S0730-725X (96) 00386-4.
G. Cole, J. Pilitsis, and G. S. Fischer, “Design of a robotic system for MRI-guided deep brain stimulation electrode placement,” in 2009 IEEE International Conference on Robotics and Automation, May 2009, pp. 4450-4456. doi: 10.1109/ROBOT.2009.5152343.
G. A. Cole, K. Harrington, H. Su, A. Camilo, J. G. Pilitsis, and G. S. Fischer, “Closed-Loop Actuated Surgical System Utilizing Real-Time In-Situ MRI Guidance,” in Experimental Robotics: The 12th International Symposium on Experimental Robotics, O. Khatib, V. Kumar, and G. Sukhatme, Eds. Berlin, Heidelberg: Springer, 2014, pp. 785-798. doi: 10.1007/978-3-642-28572-1 54.
G. S. Fischer et al., “MRI-Compatible Pneumatic Robot for Transperineal Prostate Needle Placement,” IEEE/ASME Transactions on Mechatronics, vol. 13, no. 3, pp. 295-305, June 2008, doi: 10.1109/TMECH.2008.924044.
S.-E. Song et al., “Development of a pneumatic robot for MRI-guided transperineal prostate biopsy and brachytherapy: New approaches,” in 2010 IEEE International Conference on Robotics and Automation, 2010, pp. 2580-2585. doi: 10.1109/ROBOT.2010.5509710.
H. Su et al., “Piezoelectrically Actuated Robotic System for MRI-Guided Prostate Percutaneous Therapy,” IEEE/ASME Transactions on Mechatronics, vol. 20, no. 4, pp. 1920-1932 Aug. 2015, doi: 10.1109/TMECH.2014.2359413.

What is claimed is:

1. A controlled pressure, continuous movement fluid agent delivery system, comprising:
one or more delivery lumen;
one or more fluid agent reservoir fluidly connected to the one or more delivery lumen by a tubing or fluid line;
one or more linear actuator mechanically connected to the one or more delivery lumen;
one or more pressure sensor;
one or more pump; and
a controller;
wherein the controller is configured to modulate a flow rate of a fluid agent by the one or more pump to maintain a controlled pressure in the one or more delivery lumen as measured by the one or more pressure sensor as the one or more linear actuator moves the one or more delivery lumen.

2. The system of claim 1, wherein the one or more delivery lumen comprises a fiberoptic needle configured to conduct a laser light.

3. The system of claim 1, wherein the one or more linear actuator comprises a lead screw actuated by a stepper motor.

4. The system of claim 1, wherein the one or more linear actuator comprises a Bowden cable.

5. The system of claim 1, wherein the one or more pump is selected from the group consisting of: a diaphragm pump, a gear pump, a lobe pump, a peristaltic pump, a piston pump, a variable height fluid column, and a syringe.

6. The system of claim 1, wherein the controlled pressure is constant as the one or more linear actuator moves the one or more delivery lumen.

7. A method of controlled pressure, continuous movement fluid agent delivery, comprising the steps of:
providing a fluid agent delivery system comprising one or more delivery lumen, each delivery lumen being connected to a linear actuator, a pressure sensor, a fluid agent reservoir, and a pump;
positioning the one or more delivery lumen at a starting position;
actuating the one or more delivery lumen from the starting position to an ending position by the linear actuator while the pump simultaneously administers a fluid agent at a flow rate that maintains a controlled pressure as measured by the pressure sensor; and
ceasing movement of the one or more delivery lumen at the ending position.

8. The method of claim 7, wherein the method further comprises a step of ceasing administration of the fluid agent after a delay after the one or more delivery lumen reaches the ending position.

9. The method of claim 8, wherein the delay is between about 1 second and 1 hour.

10. The method of claim 7, wherein the one or more delivery lumen is actuated from the starting position to the ending position at a rate between about 0.1 mm/minute and 100 mm/minute.

11. The method of claim 7, wherein the one or more delivery lumen is retracted from the starting position to the ending position.

12. The method of claim 7, wherein the controlled pressure is below a critical pressure or reflux pressure.

13. The method of claim 7, wherein the controlled pressure is constant during actuation of the one or more delivery lumen from the starting position to the ending position.

14. The method of claim 7, further comprising the steps of:
imaging a treatment area comprising the starting position prior to positioning the one or more delivery lumen at a starting position to create an initial image of the treatment area;
calculating a delivery volume of the fluid agent within the treatment area after ceasing movement of the one or more delivery lumen at the ending position;
overlaying the delivery volume with the initial image of the treatment area;
calculating a second starting position and second ending position from the overlaid delivery volume and treatment area to increase a size of a total delivery volume, based on the overlaid delivery volume and initial image of the treatment area;
positioning the one or more delivery lumen at the second starting position; and
actuating the one or more delivery lumen from the second starting position to the second ending position by the linear actuator while the pump simultaneously administers the fluid agent at the flow rate that maintains the controlled pressure as measured by the pressure sensor.

15. An MRI compatible convection-enhanced thermo-chemotherapy catheter system, comprising:
a needle assembly comprising one or more needle fluidly connected to a reservoir by tubing or fluid lines, a pump, and a pressure sensor configured to measure pressure at the one or more needle;
an actuating block comprising one or more linear actuator mechanically connected to the one or more needle;
a motor bank mechanically connected to the one or more linear actuator; and
a controller;
wherein the controller is configured to modulate a flow rate of a fluid agent by the pump to maintain a controlled pressure in the one or more needle as measured by the pressure sensor as the one or more linear actuator moves the one or more needle.

16. The system of claim 15, wherein the one or more needle comprises a fiberoptic needle configured to conduct a laser light.

17. The system of claim 15, wherein the one or more needle comprises a magnetic resonance visible coating.

18. The system of claim 15, wherein the needle assembly comprises a main cannula through which the one or more needle is extendable and retractable.

19. The system of claim 18, wherein the main cannula is mechanically connected to at least a first positioning mechanism configured to control a rotation, an extension, and a retraction of the main cannula.

20. The system of claim 18, wherein the main cannula is mechanically connected to at least a second positioning mechanism configured to control a trajectory angle of the main cannula.

21. The system of claim 15, wherein the motor bank comprises one or more stepper motor mechanically connected to the one or more linear actuator by one or more transmission rod, such that rotations of the one or more stepper motor are translated by the one or more transmission rod to the one or more linear actuator to move the one or more needle.

22. The system of claim 21, wherein the system further comprises one or more junction box mechanically connected between the motor bank and the one or more linear actuator, such that the one or more junction box forms a bend in a transmission path between the motor bank and the one or more linear actuator.

23. The system of claim 21, wherein each of the one or more transmission rod is removably connected to the system by a quick connect attachment.

24. The system of claim 15, wherein the motor bank is positionable at a location remote from a magnetic field of an MRI machine.
